SEQUENCE LISTING (272 sequences)

SEQ ID NO: 1 - protein, 1368 aa, Streptococcus pyogenes [sequence omitted]

SEQ ID NO: 2 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 116 aa, Sulfolobus solfataricus
ALLLDTDRVQFGPVVALNPATLLPLPEEGLQHNCLDGTGGGGVTVKFKYKGEELEVDISKIKKVWRVGKMISFTYDDNGKTGRGAVSEKDAPKELLQMLEKSGKKSGGSKRTADGS

SEQ ID NO: 5 - protein, 677 aa, unidentified
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 6 - protein, 497 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 7 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 8 - protein, 497 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 9 - protein, 132 aa, unidentified
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSA

SEQ ID NO: 10 - protein, 133 aa, Escherichia virus Qbeta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 11 - protein, 127 aa, unidentified
SKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVVQATSEDLVVNLVPLGR

SEQ ID NO: 12 - protein, 500 aa, unidentified
SRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 13 - protein, 5 aa, synthetic construct
GGGGS

SEQ ID NO: 14 - protein, 15 aa, synthetic construct
GGGGSGGGGSGGGGS

SEQ ID NO: 15 - protein, 18 aa, synthetic construct
GGSGGSGGSGGSGGSGGS

SEQ ID NO: 16 - protein, 5 aa, synthetic construct
PAPAP

SEQ ID NO: 17 - protein, 15 aa, synthetic construct
EAAAKEAAAKEAAAK

SEQ ID NO: 18 - protein, 16 aa, synthetic construct
SGSETPGTSESATPES

SEQ ID NO: 19 - protein, 5 aa, synthetic construct
EAAAK

SEQ ID NO: 20 - protein, 30 aa, synthetic construct
AHFKISGEKRPSTDPGKKAKNPKKKKKKDP

SEQ ID NO: 21 - protein, 12 aa, synthetic construct
AHRAKKMSKTHA

SEQ ID NO: 22 - protein, 14 aa, synthetic construct
ASPEYVNLPINGNG

SEQ ID NO: 23 - protein, 7 aa, synthetic construct
CTKRPRW

SEQ ID NO: 24 - protein, 16 aa, synthetic construct
DKAKRVSRNKSEKKRR

SEQ ID NO: 25 - protein, 15 aa, synthetic construct
EELRLKEELLKGIYA

SEQ ID NO: 26 - protein, 16 aa, synthetic construct
EEQLRRRKNSRLNNTG

SEQ ID NO: 27 - protein, 24 aa, synthetic construct
EVLKVIRTGKRKKKAWKRMVTKVC

SEQ ID NO: 28 - protein, 15 aa, synthetic construct
HHHHHHHHHHHHQPH

SEQ ID NO: 29 - protein, 17 aa, synthetic construct
HKKKHPDASVNFSEFSK

SEQ ID NO: 30 - protein, 9 aa, synthetic construct
HKRTKKNLS

SEQ ID NO: 31 - protein, 29 aa, synthetic construct
IINGRKLKLKKSRRRSSQTSNNSFTSRRS

SEQ ID NO: 32 - protein, 8 aa, synthetic construct
KAEQERRK

SEQ ID NO: 33 - protein, 18 aa, synthetic construct
KEKRKRREELFIEQKKRK

SEQ ID NO: 34 - protein, 14 aa, synthetic construct
KKGKDEWFSRGKKP

SEQ ID NO: 35 - protein, 15 aa, synthetic construct
KKGPSVQKRKKTNLS

SEQ ID NO: 36 - protein, 16 aa, synthetic construct
KKKTVINDLLHYKKEK

SEQ ID NO: 37 - protein, 17 aa, synthetic construct
KKNGGKGKNKPSAKIKK

SEQ ID NO: 38 - protein, 13 aa, synthetic construct
KKPKWDDFKKKKK

SEQ ID NO: 39 - protein, 9 aa, synthetic construct
KKRKKDNLS

SEQ ID NO: 40 - protein, 9 aa, synthetic construct
KKRRKRRRK

SEQ ID NO: 41 - protein, 9 aa, synthetic construct
KKRRRRARK

SEQ ID NO: 42 - protein, 7 aa, synthetic construct
KKSKRGR

SEQ ID NO: 43 - protein, 8 aa, synthetic construct
KKSRKRGS

SEQ ID NO: 44 - protein, 17 aa, synthetic construct
KKSTALSRELGKIMRRR

SEQ ID NO: 45 - protein, 17 aa, synthetic construct
KKSYQDPEIIAHSRPRK

SEQ ID NO: 46 - protein, 17 aa, synthetic construct
KKTGKNRKLKSKRVKTR

SEQ ID NO: 47 - protein, 17 aa, synthetic construct
KKVSIAGQSGKLWRWKR

SEQ ID NO: 48 - protein, 20 aa, synthetic construct
KKYENVVIKRSPRKRGRPRK

SEQ ID NO: 49 - protein, 6 aa, synthetic construct
KNKKRK

SEQ ID NO: 50 - protein, 6 aa, synthetic construct
KPKKKR

SEQ ID NO: 51 - protein, 19 aa, synthetic construct
KRAMKDDSHGNSTSPKRRK

SEQ ID NO: 52 - protein, 17 aa, synthetic construct
KRANSNLVAAYEKAKKK

SEQ ID NO: 53 - protein, 22 aa, synthetic construct
KRASEDTTSGSPPKKSSAGPKR

SEQ ID NO: 54 - protein, 16 aa, synthetic construct
KRFKRRWMVRKMKTKK

SEQ ID NO: 55 - protein, 16 aa, synthetic construct
KRGLNSSFETSPKKVK

SEQ ID NO: 56 - protein, 20 aa, synthetic construct
KRGNSSIGPNDLSKRKQRKK

SEQ ID NO: 57 - protein, 23 aa, synthetic construct
KRIHSVSLSQSQIDPSKKVKRAK

SEQ ID NO: 58 - protein, 15 aa, synthetic construct
KRKGKLKNKGSKRKK

SEQ ID NO: 59 - protein, 13 aa, synthetic construct
KRRRRRRREKRKR

SEQ ID NO: 60 - protein, 18 aa, synthetic construct
KRSNDRTYSPEEEKQRRA

SEQ ID NO: 61 - protein, 21 aa, synthetic construct
KRTVATNGDASGAHRAKKMSK

SEQ ID NO: 62 - protein, 20 aa, synthetic construct
KRVYNKGEDEQEHLPKGKKR

SEQ ID NO: 63 - protein, 18 aa, synthetic construct
KSGKAPRRRAVSMDNSNK

SEQ ID NO: 64 - protein, 19 aa, synthetic construct
KVNFLDMSLDDIIIYKELE

SEQ ID NO: 65 - protein, 15 aa, synthetic construct
KVQHRIAKKTTRRRR

SEQ ID NO: 66 - protein, 8 aa, synthetic construct
LSPSLSPL

SEQ ID NO: 67 - protein, 30 aa, synthetic construct
MDSLLMNRRKFLYQFKNVRWAKGRRETYLC

SEQ ID NO: 68 - protein, 52 aa, synthetic construct
MPQNEYIELHRKRYGYRLDYHEKKRKKESREAHERSKKAKKMIGLKAKLYHK

SEQ ID NO: 69 - protein, 10 aa, synthetic construct
MVQLRPRASR

SEQ ID NO: 70 - protein, 24 aa, synthetic construct
NNKLLAKRRKGGASPKDDPMDDIK

SEQ ID NO: 71 - protein, 20 aa, synthetic construct
NYKRPMDGTYGPPAKRHEGE

SEQ ID NO: 72 - protein, 19 aa, synthetic construct
PDTKRAKLDSSETTMVKKK

SEQ ID NO: 73 - protein, 7 aa, synthetic construct
PEKRTKI

SEQ ID NO: 74 - protein, 8 aa, synthetic construct
PGGRGKKK

SEQ ID NO: 75 - protein, 18 aa, synthetic construct
PGKMDKGEHRQERRDRPY

SEQ ID NO: 76 - protein, 11 aa, synthetic construct
PKKGDKYDKTD

SEQ ID NO: 77 - protein, 7 aa, synthetic construct
PKKKSRK

SEQ ID NO: 78 - protein, 7 aa, synthetic construct
PKKNKPE

SEQ ID NO: 79 - protein, 7 aa, synthetic construct
PKKRAKV

SEQ ID NO: 80 - protein, 9 aa, synthetic construct
PKPKKLKVE

SEQ ID NO: 81 - protein, 7 aa, synthetic construct
PKRGRGR

SEQ ID NO: 82 - protein, 9 aa, synthetic construct
PKRRLVDDA

SEQ ID NO: 83 - protein, 7 aa, synthetic construct
PKRRRTY

SEQ ID NO: 84 - protein, 6 aa, synthetic construct
PLFKRR

SEQ ID NO: 85 - protein, 7 aa, synthetic construct
PLRKAKR

SEQ ID NO: 86 - protein, 9 aa, synthetic construct
PPAKRKCIF

SEQ ID NO: 87 - protein, 8 aa, synthetic construct
PPARRRRL

SEQ ID NO: 88 - protein, 8 aa, synthetic construct
PPKKKRKV

SEQ ID NO: 89 - protein, 10 aa, synthetic construct
PPNKRMKVKH

SEQ ID NO: 90 - protein, 13 aa, synthetic construct
PPRIYPQLPSAPT

SEQ ID NO: 91 - protein, 13 aa, synthetic construct
PQRSPFPKSSVKR

SEQ ID NO: 92 - protein, 8 aa, synthetic construct
PRPRKVPR

SEQ ID NO: 93 - protein, 9 aa, synthetic construct
PRRRVQRKR

SEQ ID NO: 94 - protein, 7 aa, synthetic construct
PRRVRLK

SEQ ID NO: 95 - protein, 7 aa, synthetic construct
PSRKRPR

SEQ ID NO: 96 - protein, 8 aa, synthetic construct
PSSKKRKV

SEQ ID NO: 97 - protein, 7 aa, synthetic construct
PTKKRVK

SEQ ID NO: 98 - protein, 9 aa, synthetic construct
QRPGPYDRP

SEQ ID NO: 99 - protein, 18 aa, synthetic construct
RGKGGKGLGKGGAKRHRK

SEQ ID NO: 100 - protein, 24 aa, synthetic construct
RKAGKGGGGHKTTKKRSAKDEKVP

SEQ ID NO: 101 - protein, 9 aa, synthetic construct
RKIKLKRAK

SEQ ID NO: 102 - protein, 9 aa, synthetic construct
RKIKRKRAK

SEQ ID NO: 103 - protein, 17 aa, synthetic construct
RKKEAPGPREELRSRGR

SEQ ID NO: 104 - protein, 7 aa, synthetic construct
RKKRKGK

SEQ ID NO: 105 - protein, 9 aa, synthetic construct
RKKRRQRRR

SEQ ID NO: 106 - protein, 25 aa, synthetic construct
RKKSIPLSIKNLKRKHKRKKNKITR

SEQ ID NO: 107 - protein, 20 aa, synthetic construct
RKLVKPKNTKMKTKLRTNPY

SEQ ID NO: 108 - protein, 16 aa, synthetic construct
RKRLILSDKGQLDWKK

SEQ ID NO: 109 - protein, 7 aa, synthetic construct
RKRLKSK

SEQ ID NO: 110 - protein, 9 aa, synthetic construct
RKRRVRDNM

SEQ ID NO: 111 - protein, 22 aa, synthetic construct
RKRSPKDKKEKDLDGAGKRRKT

SEQ ID NO: 112 - protein, 22 aa, synthetic construct
RKRTPRVDGQTGENDMNKRRRK

SEQ ID NO: 113 - protein, 10 aa, synthetic construct
RLPVRRRRRR

SEQ ID NO: 114 - protein, 10 aa, synthetic construct
RLRFRKPKSK

SEQ ID NO: 115 - protein, 6 aa, synthetic construct
RQQRKR

SEQ ID NO: 116 - protein, 16 aa, synthetic construct
RRDLNSSFETSPKKVK

SEQ ID NO: 117 - protein, 8 aa, synthetic construct
RRDRAKLR

SEQ ID NO: 118 - protein, 8 aa, synthetic construct
RRGDGRRR

SEQ ID NO: 119 - protein, 11 aa, synthetic construct
RRGRKRKAEKQ

SEQ ID NO: 120 - protein, 6 aa, synthetic construct
RRKKRR

SEQ ID NO: 121 - protein, 19 aa, synthetic construct
RRKRSKSEDMDSVESKRRR

SEQ ID NO: 122 - protein, 6 aa, synthetic construct
RRKRSR

SEQ ID NO: 123 - protein, 14 aa, synthetic construct
RRPKGKTLQKRKPK

SEQ ID NO: 124 - protein, 18 aa, synthetic construct
RRRGFERFGPDNMGRKRK

SEQ ID NO: 125 - protein, 15 aa, synthetic construct
RRRGKNKVAAQNCRK

SEQ ID NO: 126 - protein, 9 aa, synthetic construct
RRRKRRNLS

SEQ ID NO: 127 - protein, 14 aa, synthetic construct
RRRQKQKGGASRRR

SEQ ID NO: 128 - protein, 13 aa, synthetic construct
RRRREGPRARRRR

SEQ ID NO: 129 - protein, 61 aa, synthetic construct
RRTIRLKLVYDKCDRSCKIQKKNRNKCQYCRFHKCLSVGMSHNAIRFGRMPRSEKAKLKAE

SEQ ID NO: 130 - protein, 17 aa, synthetic construct
RRVPQRKEVSRCRKCRK

SEQ ID NO: 131 - protein, 32 aa, synthetic construct
RVGGRRQAVECIEDLLNEPGQPLDLSCKRPRP

SEQ ID NO: 132 - protein, 9 aa, synthetic construct
RVVKLRIAP

SEQ ID NO: 133 - protein, 6 aa, synthetic construct
RVVRRR

SEQ ID NO: 134 - protein, 12 aa, synthetic construct
SKRKTKISRKTR

SEQ ID NO: 135 - protein, 16 aa, synthetic construct
SYVKTVPNRTRTYIKL

SEQ ID NO: 136 - protein, 12 aa, synthetic construct
TGKNEAKKRKIA

SEQ ID NO: 137 - protein, 30 aa, synthetic construct
TLSPASSPSSVSCPVIPASTDESPGSALNI

SEQ ID NO: 138 - protein, 2265 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGMKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 139 - DNA, 34 bp, synthetic construct
  source 1..34: mol_type = other DNA
ataacttcgtataatgtatgctatacgaacggta

SEQ ID NO: 140 - DNA, 34 bp, synthetic construct
  source 1..34: mol_type = other DNA
taccgttcgtataatgtatgctatacgaagttat

SEQ ID NO: 141 - DNA, 46 bp, synthetic construct
  source 1..46: mol_type = other DNA
ggccggcttgtcgacgacggcggtctccgtcgtcaggatcatccgg

SEQ ID NO: 142 - DNA, 46 bp, synthetic construct
  source 1..46: mol_type = other DNA
ccggatgatcctgacgacggagaccgccgtcgtcgacaagccggcc

SEQ ID NO: 143 - DNA, 38 bp, synthetic construct
  source 1..38: mol_type = other DNA
ggcttgtcgacgacggcgttctccgtcgtcaggatcat

SEQ ID NO: 144 - DNA, 52 bp, synthetic construct
  source 1..52: mol_type = other DNA
gtggtttgtctggtcaaccaccgcgttctcagtggtgtacggtacaaaccca

SEQ ID NO: 145 - DNA, 38 bp, synthetic construct
  source 1..38: mol_type = other DNA
ggcttgtcgacgacggcgaactccgtcgtcaggatcat

SEQ ID NO: 146 - DNA, 52 bp, synthetic construct
  source 1..52: mol_type = other DNA
gtggtttgtctggtcaaccaccgcgaactcagtggtgtacggtacaaaccca

SEQ ID NO: 147 - DNA, 38 bp, synthetic construct
  source 1..38: mol_type = other DNA
ggcttgtcgacgacggcgccctccgtcgtcaggatcat

SEQ ID NO: 148 - DNA, 52 bp, synthetic construct
  source 1..52: mol_type = other DNA
gtggtttgtctggtcaaccaccgcgccctcagtggtgtacggtacaaaccca

SEQ ID NO: 149 - DNA, 38 bp, synthetic construct
  source 1..38: mol_type = other DNA
ggcttgtcgacgacggcgggctccgtcgtcaggatcat

SEQ ID NO: 150 - DNA, 52 bp, synthetic construct
  source 1..52: mol_type = other DNA
gtggtttgtctggtcaaccaccgcgggctcagtggtgtacggtacaaaccca

SEQ ID NO: 151 - DNA, 38 bp, synthetic construct
  source 1..38: mol_type = other DNA
ggcttgtcgacgacggcgtgctccgtcgtcaggatcat

SEQ ID NO: 152 - DNA, 52 bp, synthetic construct
  source 1..52: mol_type = other DNA
gtggtttgtctggtcaaccaccgcgtgctcagtggtgtacggtacaaaccca

SEQ ID NO: 153 - DNA, 38 bp, synthetic construct
  source 1..38: mol_type = other DNA
ggcttgtcgacgacggcggtctccgtcgtcaggatcat

SEQ ID NO: 154 - DNA, 52 bp, synthetic construct
  source 1..52: mol_type = other DNA
gtggtttgtctggtcaaccaccgcggtctcagtggtgtacggtacaaaccca

SEQ ID NO: 155 - DNA, 38 bp, synthetic construct
  source 1..38: mol_type = other DNA
ggcttgtcgacgacggcgctctccgtcgtcaggatcat

SEQ ID NO: 156 - DNA, 52 bp, synthetic construct
  source 1..52: mol_type = other DNA
gtggtttgtctggtcaaccaccgcgctctcagtggtgtacggtacaaaccca

SEQ ID NO: 157 - DNA, 38 bp, synthetic construct
  source 1..38: mol_type = other DNA
ggcttgtcgacgacggcgcactccgtcgtcaggatcat

SEQ ID NO: 158 - DNA, 52 bp, synthetic construct
  source 1..52: mol_type = other DNA
gtggtttgtctggtcaaccaccgcgcactcagtggtgtacggtacaaaccca

SEQ ID NO: 159 - DNA, 38 bp, synthetic construct
  source 1..38: mol_type = other DNA
ggcttgtcgacgacggcgtcctccgtcgtcaggatcat

SEQ ID NO: 160 - DNA, 52 bp, synthetic construct
  source 1..52: mol_type = other DNA
gtggtttgtctggtcaaccaccgcgtcctcagtggtgtacggtacaaaccca

SEQ ID NO: 161 - DNA, 38 bp, synthetic construct
  source 1..38: mol_type = other DNA
ggcttgtcgacgacggcggactccgtcgtcaggatcat

SEQ ID NO: 162 - DNA, 52 bp, synthetic construct
  source 1..52: mol_type = other DNA
gtggtttgtctggtcaaccaccgcggactcagtggtgtacggtacaaaccca

SEQ ID NO: 163 - DNA, 38 bp, synthetic construct
  source 1..38: mol_type = other DNA
ggcttgtcgacgacggcgagctccgtcgtcaggatcat

SEQ ID NO: 164 - DNA, 52 bp, synthetic construct
  source 1..52: mol_type = other DNA
gtggtttgtctggtcaaccaccgcgagctcagtggtgtacggtacaaaccca

SEQ ID NO: 165 - DNA, 38 bp, synthetic construct
  source 1..38: mol_type = other DNA
ggcttgtcgacgacggcgacctccgtcgtcaggatcat

SEQ ID NO: 166 - DNA, 52 bp, synthetic construct
  source 1..52: mol_type = other DNA
gtggtttgtctggtcaaccaccgcgacctcagtggtgtacggtacaaaccca

SEQ ID NO: 167 - DNA, 38 bp, synthetic construct
  source 1..38: mol_type = other DNA
ggcttgtcgacgacggcgatctccgtcgtcaggatcat

SEQ ID NO: 168 - DNA, 52 bp, synthetic construct
  source 1..52: mol_type = other DNA
gtggtttgtctggtcaaccaccgcgatctcagtggtgtacggtacaaaccca

SEQ ID NO: 169 - DNA, 38 bp, synthetic construct
  source 1..38: mol_type = other DNA
ggcttgtcgacgacggcggcctccgtcgtcaggatcat

SEQ ID NO: 170 - DNA, 52 bp, synthetic construct
  source 1..52: mol_type = other DNA
gtggtttgtctggtcaaccaccgcggcctcagtggtgtacggtacaaaccca

SEQ ID NO: 171 - DNA, 38 bp, synthetic construct
  source 1..38: mol_type = other DNA
ggcttgtcgacgacggcgcgctccgtcgtcaggatcat

SEQ ID NO: 172 - DNA, 52 bp, synthetic construct
  source 1..52: mol_type = other DNA
gtggtttgtctggtcaaccaccgcgcgctcagtggtgtacggtacaaaccca

SEQ ID NO: 173 - DNA, 38 bp, synthetic construct
  source 1..38: mol_type = other DNA
ggcttgtcgacgacggcgtactccgtcgtcaggatcat

SEQ ID NO: 174 - DNA, 52 bp, synthetic construct
  source 1..52: mol_type = other DNA
gtggtttgtctggtcaaccaccgcgtactcagtggtgtacggtacaaaccca

SEQ ID NO: 175 - DNA, 45 bp, synthetic construct
  source 1..45: mol_type = other DNA
tgcgggtgccagggcgtgcccttgggctccccgggcgcgtactcc

SEQ ID NO: 176 - DNA, 42 bp, synthetic construct
  source 1..42: mol_type = other DNA
gtgccccaactggggtaacctttgagttctctcagttggggg

SEQ ID NO: 177 - DNA, 57 bp, synthetic construct
  source 1..57: mol_type = other DNA
gcgcccaagttgcccatgaccatgccgaagcagtggtagaagggcaccggcagacac

SEQ ID NO: 178 - DNA, 70 bp, synthetic construct
  source 1..70: mol_type = other DNA
aggcatgttccccaaagcgataccacttgaagcagtggtactgcttgtgggtacactctgcgggtgatga

SEQ ID NO: 179 - DNA, 60 bp, synthetic construct
  source 1..60: mol_type = other DNA
gtccttgaccaggtttttgacgaaagtgatccagatgatccagctccacaccccgaacgc

SEQ ID NO: 180 - DNA, 63 bp, synthetic construct
  source 1..63: mol_type = other DNA
ggtgctgggttgttgtctctggacagtgatccatgggaaactactcagcaccaccaatgttcc

SEQ ID NO: 181 - DNA, 50 bp, synthetic construct
  source 1..50: mol_type = other DNA
tcggccggcttgtcgacgacggcggtctccgtcgtcaggatcatccgggc

SEQ ID NO: 182 - DNA, 58 bp, synthetic construct
  source 1..58: mol_type = other DNA
gtcgtggtttgtctggtcaaccaccgcggtctcagtggtgtacggtacaaaccccgac

SEQ ID NO: 183 - DNA, 46 bp, synthetic construct
  source 1..46: mol_type = other DNA
gatcagctccgcgggcaagaccttctccttcacggggtggaaggtc

SEQ ID NO: 184 - DNA, 67 bp, synthetic construct
  source 1..67: mol_type = other DNA
tcaaccccgttccagcccaacagtgttagtctttgctcttacccagttgggcgggatagcctgcccg

SEQ ID NO: 185 - DNA, 57 bp, synthetic construct
  source 1..57: mol_type = other DNA
aacgattttcaaaggatcactgaatcaaaagtattgctcatccacgcgaaatttttc

SEQ ID NO: 186 - DNA, 57 bp, synthetic construct
  source 1..57: mol_type = other DNA
aatattttaggtatatgattttgtttattagtgtaaataacactatgtacctaaaat

SEQ ID NO: 187 - DNA, 53 bp, synthetic construct
  source 1..53: mol_type = other DNA
tgtaaaggagactgataatggcatgtacaactatactcgtcggtaaaaaggca

SEQ ID NO: 188 - DNA, 52 bp, synthetic construct
  source 1..52: mol_type = other DNA
taaaaaaatacagcgtttttcatgtacaactatactagttgtagtgcctaaa

SEQ ID NO: 189 - DNA, 56 bp, synthetic construct
  source 1..56: mol_type = other DNA
gagcgccggatcagggagtggacggcctgggagcgctacacgctgtggctgcggtc

SEQ ID NO: 190 - DNA, 56 bp, synthetic construct
  source 1..56: mol_type = other DNA
ccctaatacgcaagtcgataactctcctgggagcgttgacaacttgcgcaccctga

SEQ ID NO: 191 - DNA, 68 bp, synthetic construct
  source 1..68: mol_type = other DNA
tctcgtggtggtggaaggtgttggtgcggggttggccgtggtcgaggtggggtggtggtagccattcg

SEQ ID NO: 192 - DNA, 69 bp, synthetic construct
  source 1..69: mol_type = other DNA
gcacaggtgtagtgtatctcacaggtccacggttggccgtggactgctgaagaacattccacgccagga

SEQ ID NO: 193 - DNA, 65 bp, synthetic construct
  source 1..65: mol_type = other DNA
agtgcagcatgtcattaatatcagtacagataaagctgtatctcctgtgaacacaatgggtgcca

SEQ ID NO: 194 - DNA, 55 bp, synthetic construct
  source 1..55: mol_type = other DNA
aaagtagtaagtatcttaaaaaacagataaagctgtatattaagatacttactac

SEQ ID NO: 195 - DNA, 54 bp, synthetic construct
  source 1..54: mol_type = other DNA
tgataattgccaacacaattaacatctcaatcaaggtaaatgctttttcgtttt

SEQ ID NO: 196 - DNA, 54 bp, synthetic construct
  source 1..54: mol_type = other DNA
aattgcgagtttttatttcgtttatttcaattaaggtaactaaaaaactccttt

SEQ ID NO: 197 - DNA, 68 bp, synthetic construct
  source 1..68: mol_type = other DNA
aaggtagcgtcaacgataggtgtaactgtcgtgtttgtaacggtacttccaacagctggcgtttcagt

SEQ ID NO: 198 - DNA, 68 bp, synthetic construct
  source 1..68: mol_type = other DNA
tagttttaaagttggttattagttactgtgatatttatcacggtacccaataaccaatgaatatttga

SEQ ID NO: 199 - DNA, 57 bp, synthetic construct
  source 1..57: mol_type = other DNA
tgtaactttttcggatcaagctatgaaggacgcaaagagggaactaaacacttaatt

SEQ ID NO: 200 - DNA, 57 bp, synthetic construct
  source 1..57: mol_type = other DNA
ttgtttagttcctcgttttctctcgttggaagaagaagaaacgagaaactaaaatta

SEQ ID NO: 201 - DNA, 63 bp, synthetic construct
  source 1..63: mol_type = other DNA
caacctgttgacatgtttccacagacaactcacgtggaggtagtcacggcttttacgttagtt

SEQ ID NO: 202 - DNA, 61 bp, synthetic construct
  source 1..61: mol_type = other DNA
gagaatactgttgaacaatgaaaaactaggcatgtagaagttgtttgtgcactaactttaa

SEQ ID NO: 203 - DNA, 120 bp, synthetic construct
  source 1..120: mol_type = other DNA
acaggtcaacacatcgcagttatcgaacaatcttcgaaaatgtatggaggcacttgtatcaatataggatgtataccttcgaagacacttgtacatgatggattagaaggcaaatccttt

SEQ ID NO: 204 - DNA, 120 bp, synthetic construct
  source 1..120: mol_type = other DNA
caaaataaaaaacattgatttttattaacttcttttgtgcggaactacgaacagttcattaatacgaagtgtacaaacttccatacaaaaataaccacgacaattaagacgtggtttcta

SEQ ID NO: 205 - DNA, 17 bp, synthetic construct
  source 1..17: mol_type = other DNA
attatttctcaccctga

SEQ ID NO: 206 - DNA, 17 bp, synthetic construct
  source 1..17: mol_type = other DNA
atcatctcccacccgga

SEQ ID NO: 207 - DNA, 34 bp, synthetic construct
  source 1..34: mol_type = other DNA
aataggtctgagaacgcccattctcagacgtatt

SEQ ID NO: 208 - DNA, 34 bp, synthetic construct
  source 1..34: mol_type = other DNA
gaagttcctatactttctagagaataggaacttc

SEQ ID NO: 209 - DNA, 46 bp, synthetic construct
  source 1..46: mol_type = other DNA
ggccggcttgtcgacgacggcgaactccgtcgtcaggatcatccgg

SEQ ID NO: 210 - DNA, 46 bp, synthetic construct
  source 1..46: mol_type = other DNA
ggccggcttgtcgacgacggcggactccgtcgtcaggatcatccgg

SEQ ID NO: 211 - DNA, 46 bp, synthetic construct
  source 1..46: mol_type = other DNA
ggccggcttgtcgacgacggcgcactccgtcgtcaggatcatccgg

SEQ ID NO: 212 - DNA, 46 bp, synthetic construct
  source 1..46: mol_type = other DNA
ggccggcttgtcgacgacggcgtactccgtcgtcaggatcatccgg

SEQ ID NO: 213 - DNA, 46 bp, synthetic construct
  source 1..46: mol_type = other DNA
ggccggcttgtcgacgacggcgagctccgtcgtcaggatcatccgg

SEQ ID NO: 214 - DNA, 46 bp, synthetic construct
  source 1..46: mol_type = other DNA
ggccggcttgtcgacgacggcgggctccgtcgtcaggatcatccgg

SEQ ID NO: 215 - DNA, 46 bp, synthetic construct
  source 1..46: mol_type = other DNA
ggccggcttgtcgacgacggcgcgctccgtcgtcaggatcatccgg

SEQ ID NO: 216 - DNA, 46 bp, synthetic construct
  source 1..46: mol_type = other DNA
ggccggcttgtcgacgacggcgtgctccgtcgtcaggatcatccgg

SEQ ID NO: 217 - DNA, 46 bp, synthetic construct
  source 1..46: mol_type = other DNA
ggccggcttgtcgacgacggcgacctccgtcgtcaggatcatccgg

SEQ ID NO: 218 - DNA, 46 bp, synthetic construct
  source 1..46: mol_type = other DNA
ggccggcttgtcgacgacggcggcctccgtcgtcaggatcatccgg

SEQ ID NO: 219 - DNA, 46 bp, synthetic construct
  source 1..46: mol_type = other DNA
ggccggcttgtcgacgacggcgccctccgtcgtcaggatcatccgg

SEQ ID NO: 220 - DNA, 46 bp, synthetic construct
  source 1..46: mol_type = other DNA
ggccggcttgtcgacgacggcgtcctccgtcgtcaggatcatccgg

SEQ ID NO: 221 - DNA, 46 bp, synthetic construct
  source 1..46: mol_type = other DNA
ggccggcttgtcgacgacggcgatctccgtcgtcaggatcatccgg

SEQ ID NO: 222 - DNA, 46 bp, synthetic construct
  source 1..46: mol_type = other DNA
ggccggcttgtcgacgacggcgctctccgtcgtcaggatcatccgg

SEQ ID NO: 223 - DNA, 46 bp, synthetic construct
  source 1..46: mol_type = other DNA
ggccggcttgtcgacgacggcgttctccgtcgtcaggatcatccgg

SEQ ID NO: 224 - DNA, 38 bp, synthetic construct; identical to SEQ ID NO: 153
  source 1..38: mol_type = other DNA

SEQ ID NO: 225 - DNA, 38 bp, synthetic construct; identical to SEQ ID NO: 145
  source 1..38: mol_type = other DNA

SEQ ID NO: 226 - DNA, 38 bp, synthetic construct; identical to SEQ ID NO: 161
  source 1..38: mol_type = other DNA

SEQ ID NO: 227 - DNA, 38 bp, synthetic construct; identical to SEQ ID NO: 157
  source 1..38: mol_type = other DNA

SEQ ID NO: 228 - DNA, 38 bp, synthetic construct; identical to SEQ ID NO: 173
  source 1..38: mol_type = other DNA

SEQ ID NO: 229 - DNA, 38 bp, synthetic construct; identical to SEQ ID NO: 163
  source 1..38: mol_type = other DNA

SEQ ID NO: 230 - DNA, 38 bp, synthetic construct; identical to SEQ ID NO: 149
  source 1..38: mol_type = other DNA

SEQ ID NO: 231 - DNA, 38 bp, synthetic construct; identical to SEQ ID NO: 171
  source 1..38: mol_type = other DNA

SEQ ID NO: 232 - DNA, 38 bp, synthetic construct; identical to SEQ ID NO: 151
  source 1..38: mol_type = other DNA

SEQ ID NO: 233 - DNA, 38 bp, synthetic construct; identical to SEQ ID NO: 165
  source 1..38: mol_type = other DNA

SEQ ID NO: 234 - DNA, 38 bp, synthetic construct; identical to SEQ ID NO: 169
  source 1..38: mol_type = other DNA

SEQ ID NO: 235 - DNA, 38 bp, synthetic construct; identical to SEQ ID NO: 147
  source 1..38: mol_type = other DNA

SEQ ID NO: 236 - DNA, 38 bp, synthetic construct; identical to SEQ ID NO: 159
  source 1..38: mol_type = other DNA

SEQ ID NO: 237 - DNA, 38 bp, synthetic construct; identical to SEQ ID NO: 167
  source 1..38: mol_type = other DNA

SEQ ID NO: 238 - DNA, 38 bp, synthetic construct; identical to SEQ ID NO: 155
  source 1..38: mol_type = other DNA

SEQ ID NO: 239 - DNA, 38 bp, synthetic construct; identical to SEQ ID NO: 143
  source 1..38: mol_type = other DNA

SEQ ID NO: 240 - DNA, 34 bp, synthetic construct; identical to SEQ ID NO: 140
  source 1..34: mol_type = other DNA

SEQ ID NO: 241 - DNA, 34 bp, synthetic construct; identical to SEQ ID NO: 139
  source 1..34: mol_type = other DNA

SEQ ID NO: 242 - DNA, 27 bp, synthetic construct
  source 1..27: mol_type = other DNA
tttaccttgattgagatgttaattgtg

SEQ ID NO: 243 - DNA, 50 bp, synthetic construct
  source 1..50: mol_type = other DNA
gcgagtttttatttcgtttatttcaattaaggtaactaaaaaactccttt

SEQ ID NO: 244 - DNA, 34 bp, synthetic construct
  source 1..34: mol_type = other DNA
ctggatcatctggatcactttcgtcaaaaacctg

SEQ ID NO: 245 - DNA, 57 bp, synthetic construct
  source 1..57: mol_type = other DNA
ttcgggtgctgggttgttgtctctggacagtgatccatgggaaactactcagcacca

SEQ ID NO: 246 - DNA, 38 bp, synthetic construct
  source 1..38: mol_type = other DNA
ccccaactggggtaacctttgagttctctcagttgggg

SEQ ID NO: 247 - DNA, 21 bp, unidentified
  source 1..21: mol_type = unassigned DNA
aacatgaggatcacccatgtc

SEQ ID NO: 248 - DNA, 20 bp, Escherichia virus Qbeta
  source 1..20: mol_type = genomic DNA
atgcatgtctaagacagcat

SEQ ID NO: 249 - DNA, 25 bp, unidentified
  source 1..25: mol_type = unassigned DNA
taaggagtttatatggaaaccctta

SEQ ID NO: 250 - DNA, 109 bp, synthetic construct
  source 1..109: mol_type = other DNA
aacatgaggatcacccatgtcgacgagcgcggcgatatcatcatccatggccggatgatcctgacgacggagaccgccgtcgtcgacaagccggcctgagctgcgagaa

SEQ ID NO: 251 - DNA, 88 bp, synthetic construct
  source 1..88: mol_type = other DNA
aacatgaggatcacccatgtccgggggtcgcagtcgccatgatgatcctgacgacggagaccgccgtcgtcgacaagcccgggcggcg

SEQ ID NO: 252 - DNA, 109 bp, synthetic construct
  source 1..109: mol_type = other DNA
gacgagcgcggcgatatcatcatccatggccggatgatcctgacgacggagaccgccgtcgtcgacaagccggcctgagctgcgagaaaacatgaggatcacccatgtc

SEQ ID NO: 253 - DNA, 88 bp, synthetic construct
  source 1..88: mol_type = other DNA
cgggggtcgcagtcgccatgatgatcctgacgacggagaccgccgtcgtcgacaagcccgggcggcgaacatgaggatcacccatgtc

SEQ ID NO: 254 - DNA, 130 bp, synthetic construct
  source 1..130: mol_type = other DNA
aacatgaggatcacccatgtcgacgagcgcggcgatatcatcatccatggccggatgatcctgacgacggagaccgccgtcgtcgacaagccggcctgagctgcgagaaaacatgaggatcacccatgtc

SEQ ID NO: 255 - DNA, 109 bp, synthetic construct
  source 1..109: mol_type = other DNA
aacatgaggatcacccatgtccgggggtcgcagtcgccatgatgatcctgacgacggagaccgccgtcgtcgacaagcccgggcggcgaacatgaggatcacccatgtc

SEQ ID NO: 256 - DNA, 88 bp, synthetic construct
  source 1..88: mol_type = other DNA
gacgagcgcggcgatatcatcatccatggccggatgatcctgacgacggagaccgccgtcgtcgacaagccggcctgagctgcgagaa

SEQ ID NO: 257 - DNA, 67 bp, synthetic construct
  source 1..67: mol_type = other DNA
cgggggtcgcagtcgccatgatgatcctgacgacggagaccgccgtcgtcgacaagcccgggcggcg

SEQ ID NO: 258 - DNA, 109 bp, synthetic construct; identical to SEQ ID NO: 250
  source 1..109: mol_type = other DNA

SEQ ID NO: 259 - DNA, 88 bp, synthetic construct; identical to SEQ ID NO: 251
  source 1..88: mol_type = other DNA

SEQ ID NO: 260 - DNA, 109 bp, synthetic construct; identical to SEQ ID NO: 252
  source 1..109: mol_type = other DNA

SEQ ID NO: 261 - DNA, 88 bp, synthetic construct; identical to SEQ ID NO: 253
  source 1..88: mol_type = other DNA

SEQ ID NO: 262 - DNA, 130 bp, synthetic construct; identical to SEQ ID NO: 254
  source 1..130: mol_type = other DNA

SEQ ID NO: 263 - DNA, 109 bp, synthetic construct; identical to SEQ ID NO: 255
  source 1..109: mol_type = other DNA

SEQ ID NO: 264 - DNA, 6288 bp, synthetic construct
  source 1..6288: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccc

SEQ ID NO: 265 - DNA, 6732 bp, synthetic construct
  source 1..6732: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggaatgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccc

SEQ ID NO: 266 - DNA, 96 bp, synthetic construct
  source 1..96: mol_type = other DNA
gaagccggccttgcacatgcgttttagagctagaaatagcaagttaaaataaggctagtccgttatcaacttgaaaaagtggcaccgagtcggtgc

SEQ ID NO: 267 - DNA, 96 bp, synthetic construct
  source 1..96: mol_type = other DNA
gctattctcgcagctcaccagttttagagctagaaatagcaagttaaaataaggctagtccgttatcaacttgaaaaagtggcaccgagtcggtgc

SEQ ID NO: 268 - DNA, 96 bp, synthetic construct
  source 1..96: mol_type = other DNA
gcgtggtggggccgccagcggttttagagctagaaatagcaagttaaaataaggctagtccgttatcaacttgaaaaagtggcaccgagtcggtgc

SEQ ID NO: 269 - DNA, 96 bp, synthetic construct
  source 1..96: mol_type = other DNA
gctgtctccgccgcccgccagttttagagctagaaatagcaagttaaaataaggctagtccgttatcaacttgaaaaagtggcaccgagtcggtgc

SEQ ID NO: 270 - DNA, 8055 bp, synthetic construct
  source 1..8055: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagctctggtagcgagacacccggtaccagtgaaagcgccacgccagaaagcagtgggagtgagactccgggtacatctgaatcagcgacaccggaatcaagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggcccccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtcggggggtcaggtggatccggcggaagtggcggatccggtggatctggcggcagtccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtcctag

SEQ ID NO: 271 - DNA, 8778 bp, synthetic construct
  source 1..8778: mol_type = other DNA
atgttattaattaacgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgccatggccagcaacttcacccagttcgtgctggtggacaacggcggcaccggcgacgtgaccgtggcccccagcaacttcgccaacggcatcgccgagtggatcagcagcaacagcagaagccaggcctacaaggtgacctgcagcgtgagacagagcagcgcccagaacagaaagtacaccatcaaggtggaggtgcccaagggcgcctggagaagctacctgaacatggagctgaccatccccatcttcgccaccaacagcgactgcgagctgatcgtgaaggccatgcagggcctgctgaaggacggcaaccccatccccagcgccatcgccgccaacagcggcatctacgccgacatgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagctctggtagcgagacacccggtaccagtgaaagcgccacgccagaaagcagtgggagtgagactccgggtacatctgaatcagcgacaccggaatcaagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggcccccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtcggggggtcaggtggatccggcggaagtggcggatccggtggatctggcggcagtccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtcctag

SEQ ID NO: 272 - DNA, 875 bp, synthetic construct
  source 1..875: mol_type = other DNA
gtggtttgtctggtcaaccaccgcggtctcagtggtgtacggtacaaacccagctaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtgggcccgccccaactggggtaacctttgagttctctcagttgggg

(19) United States
(12) Patent Application Publication
(10) Pub. No.: US 2023/0383274 A1
(43) Pub. Date: Nov. 30, 2023

(54) SITE SPECIFIC GENETIC ENGINEERING UTILIZING TRANS-TEMPLATE RNAS

(71) Applicant: Massachusetts Institute of Technology, Cambridge, MA (US)

(72) Inventors: Omar Abudayyeh, Cambridge, MA (US); Jonathan Gootenberg, Cambridge, MA (US); Kaiyi Jiang, Cambridge, MA (US)

(21) Appl. No.: 18/303,533

(22) Filed: Apr. 19, 2023

Related U.S. Application Data
(60) Provisional application No. 63/363,259, filed on Apr. 20, 2022.

(51) Int. Cl. C12N 9/22; C12N 15/63; C12N 15/10

(52) U.S. Cl. CPC C12N 9/22; C12N 15/63; C12N 15/102

(57) ABSTRACT

Provided herein are compositions comprising, inter alia, a ttRNAs comprising (i) a primer binding site, (ii) a reverse transcriptase template sequence, (iii) an aptamer, and (iv) an integration sequence comprising an integration site. Also described herein are method of use of the ttRNAs in methods of editing and integrating polynucleotide sequences.

### CROSS REFERENCE TO RELATED APPLICATION

This application claims the benefit of U.S. Provisional Patent Application Ser. No. 63/363,259, filed Apr. 20, 2022. The content of the above-referenced patent application is herein incorporated by reference in its entirety.

### STATEMENT AS TO FEDERALLY FUNDED RESEARCH

This invention was made with government support under EB031957 and AI49694 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Apr. 11, 2023, is named 740489_083474-037_SL.xml and is 263,356 bytes in size.

### FIELD

This disclosure relates to non-naturally occurring systems and compositions for site specific genetic engineering comprising the use of trans-template gRNAs (e.g., described herein) in addition to targeting gRNAs. The disclosure also relates to methods of using said systems and compositions for e.g., the treatment of diseases.

### BACKGROUND

Traditional CRISPR-Cas-based engineering systems enable editing of double stranded polynucleotides, through the introduction of a double strand break in the genome of the target cell. The resolution of these double strand breaks by the cellular repair mechanisms is known to be highly error prone, raising safety concerns regarding the use the technology for therapeutic uses. Like traditional CRISPR-Cas systems, the PRIME engineering system can also mediate editing of double stranded genomes, but requires only a single strand break in the genome of the target cell, avoiding some of the safety concerns associated with the traditional-CRISPR-Cas systems. While, for this reason, PRIME editing is advantageous compared to traditional CRISPR-Cas systems, the PRIME system remains inefficient, with significant bottlenecks in large scale manufacturing that would be required for therapeutic use. Therefore, there is a need for more effective tools for gene editing and delivery.

### SUMMARY

The present disclosure provides compositions and systems for site-specific integration of exogenous polynucleotides that utilize a standard targeting gRNA combined with a trans-template RNA (ttRNA) that comprises a primer binding site, a reverse transcription template, an aptamer, and an editing polypeptide comprising a DNA binding nickase, a reverse transcriptase, an aptamer binding protein; and an integrase.

In one aspect, provided herein are polynucleotides encoding an editing polypeptide that comprises (i) a DNA binding nickase (or a functional fragment or variant thereof), (ii) a reverse transcriptase (or a functional fragment or variant thereof), (iii) an aptamer binding protein (or functional fragment or variant thereof); and (iv) an integrase (or a functional fragment or variant thereof); wherein each of the DNA binding nickase, reverse transcriptase, aptamer binding protein, and integrase, are each operably connected in any order.

In some embodiments, the polynucleotide is RNA or DNA.

In some embodiments, each of the DNA binding nickase, reverse transcriptase, aptamer binding protein, and integrase are each operably connected via a linker.

In some embodiments, each of the DNA binding nickase, reverse transcriptase, aptamer binding protein, and integrase are encoded in the following order from 5′ to 3′: the aptamer binding protein, the DNA binding nickase, the reverse transcriptase, and the integrase.

In some embodiments, the aptamer binding protein is an MS2 coat protein (MCP), a Qβ coat protein, or a PP7 coat protein, or a functional fragment or variant thereof. In some embodiments, the aptamer binding protein is MCP or a functional fragment or variant thereof.

In some embodiments, the integrase is Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), or Minos, or any functional fragments or variants thereof.

In some embodiments, the DNA binding nickase is a Cas9-D10A, a Cas9-H840A, a Cas12a nickase, or a Cas12b nickase, or a functional fragment or variant thereof.

In some embodiments, the reverse transcriptase is derived from a Moloney Murine Leukemia Virus (M-MLV) reverse transcriptase domain, transcription xenopolymerase (RTX), avian myeloblastosis virus reverse transcriptase (AMV-RT), or Eubacterium rectale maturase RT (MarathonRT).

In one aspect, provided herein are vectors comprising a polynucleotide described herein. In some embodiments, the vector is a viral vector or a plasmid.

In one aspect, the vector is a viral vector or a plasmid.

In one aspect, provided herein are particles comprising a polynucleotide described herein, a vector described herein, or a polypeptide described herein. In some embodiments, the particle is a lipid nanoparticle or a viral particle.

In one aspect, provided herein are cells comprising a polynucleotide described herein, a vector described herein, a polypeptide described herein, or a particle described herein.

In one aspect, provided herein are pharmaceutical compositions comprising a polynucleotide described herein, a vector described herein, a polypeptide described herein, or a particle described herein; and a pharmaceutically acceptable excipient.

In one aspect, provided herein are kits comprising a polynucleotide described herein, a vector described herein, a polypeptide described herein, a particle described herein, or a pharmaceutical composition described herein, and instructions for use.

In one aspect, provided herein are RNA polynucleotides comprising (i) a primer binding site, (ii) a reverse transcription template sequence that comprises an integration recognition sequence, and (iii) at least one aptamer.

In some embodiments, the aptamer is an MS2 aptamer, a Qβ RNA aptamer, or a PP7 RNA aptamer. In some embodiments, the aptamer is an MS2 aptamer.

In some embodiments, the integration recognition sequence comprises an attB site, an attP site, an attL site, an attR site, a Vox site, or a FRT site.

In one aspect, provided herein are DNA polynucleotides encoding an RNA polynucleotide described herein.

In one aspect, provided herein are vectors comprising a DNA polynucleotide described herein. In some embodiments, the vector is a viral vector or a plasmid.

In one aspect, provided herein are particles comprising an RNA polynucleotide described herein, a DNA polynucleotide described herein, or a vector described herein. In some embodiments, the particle is a lipid nanoparticle or a viral particle.

In one aspect, provided herein are cells comprising an RNA polynucleotide described herein, a DNA polynucleotide described herein, a vector described herein, or a particle described herein.

In one aspect, provided herein are pharmaceutical compositions comprising an RNA polynucleotide described herein, a DNA polynucleotide described herein, a vector described herein, or a particle described herein, and a pharmaceutically acceptable excipient.

In one aspect, provided herein are kits comprising an RNA polynucleotide described herein, a DNA polynucleotide described herein, a vector described herein, or a particle described herein, or a pharmaceutical composition described herein.

In one aspect, provided herein are compositions comprising (a) the polynucleotide described herein, a vector comprising a polynucleotide described herein, a polypeptide encoded by a polynucleotide described herein, or a particle comprising the polynucleotide, vector or polypeptide; and (b) RNA polynucleotide described herein, a DNA polynucleotide encoding an RNA polynucleotide described herein, a vector comprising the RNA or DNA polynucleotide, or a particle comprising the RNA polynucleotide, DNA polynucleotide, or vector.

In some embodiments, the composition further comprises (c) at least one targeting guide RNA (gRNA) that comprises (i) a spacer and (ii) a scaffold. In some embodiments, the composition further comprises (c) a plurality of targeting gRNAs each comprising (i) a spacer and (ii) a scaffold.

In some embodiments, the composition further comprises (c) at least two, three, four, five, six, seven, eight, nine, or ten targeting gRNAs each comprising (i) a spacer and (ii) a scaffold, wherein each of the at least two, three, four, five, six, seven, eight, nine, or ten targeting gRNAs comprises a spacer that mediates binding to the protospacer in a different target nucleic acid.

In some embodiments, the composition further comprises (d) a nicking gRNA (ngRNA).

In some embodiments, the composition further comprises a pharmaceutically acceptable excipient.

In one aspect, provided herein is system comprising (a) the polynucleotide described herein, a vector comprising a polynucleotide described herein, a polypeptide encoded by a polynucleotide described herein, or a particle comprising the polynucleotide, vector or polypeptide; and (b) RNA polynucleotide described herein, a DNA polynucleotide encoding an RNA polynucleotide described herein, a vector comprising the RNA or DNA polynucleotide, or a particle comprising the RNA polynucleotide, DNA polynucleotide, or vector.

In some embodiments, the composition further comprises (c) at least one targeting guide RNA (gRNA) that comprises (i) a spacer and (ii) a scaffold. In some embodiments, the composition further comprises (c) a plurality of targeting gRNAs each comprising (i) a spacer and (ii) a scaffold.

In some embodiments, the composition further comprises (c) at least two, three, four, five, six, seven, eight, nine, or ten targeting gRNAs each comprising (i) a spacer and (ii) a scaffold, wherein each of the at least two, three, four, five, six, seven, eight, nine, or ten targeting gRNAs comprises a spacer that mediates binding to the protospacer in a different target nucleic acid.

In some embodiments, the composition further comprises (d) a nicking gRNA (ngRNA).

In some embodiments, the composition further comprises a pharmaceutically acceptable excipient.

In one aspect, provided herein is a kit comprising (a) the polynucleotide described herein, a vector comprising a polynucleotide described herein, a polypeptide encoded by a polynucleotide described herein, or a particle comprising the polynucleotide, vector or polypeptide; and (b) RNA polynucleotide described herein, a DNA polynucleotide encoding an RNA polynucleotide described herein, a vector comprising the RNA or DNA polynucleotide, or a particle comprising the RNA polynucleotide, DNA polynucleotide, or vector; and (c) technical instructions for use.

In one aspect, provided herein are methods of site-specifically integrating a polynucleotide of interest into a target dsDNA polynucleotide, the method comprising: (1) incorporating an integration recognition sequence into a target location in the target dsDNA polynucleotide by contacting the target dsDNA polynucleotide with: (a) an editing polypeptide comprising (i) a DNA binding nickase (or a functional fragment or variant thereof), (ii) a reverse transcriptase (or a functional fragment or variant thereof), (iii) an aptamer binding protein (or a functional fragment or variant thereof), and (iv) an integrase (or a functional fragment or variant thereof), wherein each of the DNA binding nickase, reverse transcriptase, aptamer binding protein, and integrase are each operably connected in any order; (b) a targeting gRNA comprising (i) a spacer and (ii) a scaffold; and (c) a ttRNA comprising (i) a primer binding site, (ii) a reverse transcription template sequence that comprises an integration sequence that comprises an integration recognition sequence, and (iii) an aptamer; wherein the editing polypeptide's DNA binding nickase nicks a strand of the target dsDNA polynucleotide, and the reverse transcriptase reverse transcribes the reverse transcription template sequence into an extended sequence that encodes the first integration recognition sequence or a complement thereof and the extended sequence is incorporated into the target location in the target dsDNA polynucleotide; and (2) integrating the polynucleotide of interest into the target dsDNA polynucleotide, the method comprising: contacting the target dsDNA polynucleotide with a polynucleotide that comprises the polynucleotide of interest operably connected to a polynucleotide that comprises a sequence complementary or associated to the integration recognition sequence; wherein the integrase incorporates the polynucleotide of interest into the target dsDNA polynucleotide by integration, recombination, or reverse transcription of the sequence that is complementary or associated to the integration recognition sequence, to thereby site-specifically integrate the polynucleotide of interest into the target dsDNA polynucleotide.

In some embodiments, the aptamer binding protein is an MS2 coat protein (MCP), a Qβ coat protein, or a PP7 coat protein, or a functional fragment or variant thereof. In some embodiments, the aptamer binding protein is MCP or a functional fragment or variant thereof.

In some embodiments, each of the DNA binding nickase, reverse transcriptase, aptamer binding protein, and the integrase are each operably connected in any order each via a linker.

In some embodiments, each of the DNA binding nickase, reverse transcriptase, aptamer binding protein, and integrase are encoded in the following order from 5′ to 3′: the aptamer binding protein, the DNA binding nickase, the reverse transcriptase, and the integrase.

In some embodiments, the integrase is Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), or Minos, or any functional fragments or variants thereof.

In some embodiments, the DNA binding nickase is a Cas9-D10A, a Cas9-H840A, a Cas12a, or a Cas12b nickase, or a functional fragment or variant thereof.

In some embodiments, the reverse transcriptase is derived from a Moloney Murine Leukemia Virus (M-MLV) reverse transcriptase domain, transcription xenopolymerase (RTX), avian myeloblastosis virus reverse transcriptase (AMV-RT), or Eubacterium rectale maturase RT (MarathonRT).

In some embodiments, the aptamer is an MS2 aptamer, a Qβ aptamer, or a PP7 aptamer. In some embodiments, the aptamer is an MS2 aptamer.

In some embodiments, the ttRNA comprises the RNA polynucleotide described herein.

In some embodiments, the polynucleotide of interest comprises one or more nucleotide modification (e.g., insertion, deletion or substitution) compared to the endogenous sequence of the target dsDNA polynucleotide. In some embodiments, the one or more nucleotide modification is an insertion of from about 1-50, 1-40, 1-30, 1-20, 1-10, 1-5, or 1-2 nucleotides. In some embodiments, the one or more nucleotide modification is an insertion of 1, 2, 3, 4, 5, 6, 7, 8, 9, 10, 11, 12, 13, 14, 15, 16, 17, 18, 19, 20, 30, 40, or 50 nucleotides. In some embodiments, the one or more nucleotide modification is deletion of from about 1-50, 1-1-30, 1-20, 1-10, 1-5, or 1-2 nucleotides. In some embodiments, the one or more nucleotide modification is a deletion of 1, 2, 3, 4, 5, 6, 7, 8, 9, 10, 11, 12, 13, 14, 15, 16, 17, 18, 19, 20, 40, or 50 nucleotides. In some embodiments, the one or more nucleotide modification is a substitution of from about 1-10, 1-9, 1-8, 1-7, 1-6, 1-5, 1-4, 1-3, or 1-2 nucleotides. In some embodiments, the one or more nucleotide modification is a substitution of 1, 2, 3, 4, 5, 6, 7, 8, 9, or 10 nucleotides. In some embodiments, the one or more nucleotide modification is a one nucleotide substitution. In some embodiments, the one or more nucleotide modification is made in a gene associated with a disease. In some embodiments, the one or more nucleotide modification is made in a gene associated with an inherited disease.

In some embodiments, the method further comprises contacting the dsDNA polynucleotide with a ngRNA.

In some embodiments, (i) the editing polypeptide's DNA binding nickase nicks strand of the target dsDNA polynucleotide generating a free single-stranded DNA (ssDNA) polynucleotide having a 3′ end; (ii) the ssDNA hybridizes to the primer binding site of the ttRNA; (iii) the reverse transcriptase reverse transcribes a strand of DNA from the 3′ end of said ssDNA using the reverse transcription template sequence as a template, thereby generating an extended sequence comprising a ssDNA flap that encodes the integration recognition sequence; and (iv) replacing the endogenous DNA strand immediately adjacent downstream of the nick on the target strand with the ssDNA flap, thereby installing the integration recognition sequence in the target dsDNA polynucleotide.

In some embodiments, the target dsDNA polynucleotide is within the genome of a cell.

In one aspect, provided herein are methods of site specifically integrating a polynucleotide of interest into a target dsDNA polynucleotide, the method comprising (1) incorporating an integration recognition sequence into a target location in the target dsDNA polynucleotide comprising contacting the target dsDNA polynucleotide with the composition described herein or a system described herein; and (2) integrating the polynucleotide of interest into the target dsDNA polynucleotide, the method comprising contacting the target dsDNA polynucleotide with the polynucleotide of interest that comprises the polynucleotide of interest operably connected to a polynucleotide that comprises a sequence complementary or associated to the integration recognition sequence; wherein the integrase incorporates the polynucleotide of interest into the target dsDNA polynucleotide by integration, recombination, or reverse transcription of the sequence that is complementary or associated to the integration recognition sequence.

In one aspect, provided herein are methods of site-specifically integrating of a polynucleotide of interest into a target dsDNA polynucleotide, the method comprising: (1) incorporating an integration recognition sequence into a target location in the target dsDNA polynucleotide by contacting the target dsDNA polynucleotide with: (a) an editing polypeptide comprising (i) a DNA binding nickase (or a functional fragment or variant thereof), (ii) a reverse transcriptase (or a functional fragment or variant thereof), and (iii) an aptamer binding protein (or a functional fragment or variant thereof), wherein each of the DNA binding nickase, reverse transcriptase, and aptamer binding protein, are each operably connected in any order; (b) a targeting gRNA comprising (i) a spacer and (ii) a scaffold; and (c) a ttRNA comprising (i) a primer binding site, (ii) a reverse transcription template sequence that comprises an integration sequence that comprises an integration recognition sequence, and (iii) an aptamer; wherein the editing polypeptide's DNA binding nickase nicks a strand of the target dsDNA polynucleotide, and the reverse transcriptase reverse transcribes the reverse transcription template sequence into an extended sequence that encodes the first integration recognition sequence or a complement thereof and the extended sequence is incorporated into the target location in the target dsDNA polynucleotide; and (2) integrating the polynucleotide of interest into the target dsDNA polynucleotide, the method comprising: contacting the target dsDNA polynucleotide with an integrase (or a functional fragment or variant thereof) and a polynucleotide that comprises the polynucleotide of interest operably connected to a polynucleotide that comprises a sequence complementary or associated to the integration recognition sequence; wherein the integrase incorporates the polynucleotide of interest into the target dsDNA polynucleotide by integration, recombination, or reverse transcription of the sequence that is complementary or associated to the integration recognition sequence, to thereby site-specifically integrate the polynucleotide of interest into the target dsDNA polynucleotide.

In some embodiments, the aptamer binding protein is an MS2 coat protein (MCP), a Qβ coat protein, or a PP7 coat protein, or a functional fragment or variant thereof. In some embodiments, the aptamer binding protein is MCP or a functional fragment or variant thereof.

In some embodiments, each of the DNA binding nickase, reverse transcriptase, aptamer binding protein, and the integrase are each operably connected in any order each via a linker.

In some embodiments, each of the DNA binding nickase, reverse transcriptase, aptamer binding protein, and integrase are encoded in the following order from 5′ to 3′: the aptamer binding protein, the DNA binding nickase, the reverse transcriptase, and the integrase.

In some embodiments, the integrase is Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), or Minos, or any functional fragments or variants thereof.

In some embodiments, the DNA binding nickase is a Cas9-D10A, a Cas9-H840A, a Cas12a, or a Cas12b nickase, or a functional fragment or variant thereof.

In some embodiments, the reverse transcriptase is derived from a Moloney Murine Leukemia Virus (M-MLV) reverse transcriptase domain, transcription xenopolymerase (RTX), avian myeloblastosis virus reverse transcriptase (AMV-RT), or Eubacterium rectale maturase RT (MarathonRT).

In some embodiments, the aptamer is an MS2 aptamer, a Qβ aptamer, or a PP7 aptamer. In some embodiments, the aptamer is an MS2 aptamer.

In some embodiments, the ttRNA comprises the RNA polynucleotide described herein.

In some embodiments, the polynucleotide of interest comprises one or more nucleotide modification (e.g., insertion, deletion, or substitution) compared to the endogenous sequence of the target dsDNA polynucleotide. In some embodiments, the one or more nucleotide modification is an insertion of from about 1-50, 1-40, 1-30, 1-20, 1-10, 1-5, or 1-2 nucleotides. In some embodiments, the one or more nucleotide modification is an insertion of 1, 2, 3, 4, 5, 6, 7, 8, 9, 10, 11, 12, 13, 14, 15, 16, 17, 18, 19, 20, 30, 40, or 50 nucleotides. In some embodiments, the one or more nucleotide modification is deletion of from about 1-50, 1-1-30, 1-20, 1-10, 1-5, or 1-2 nucleotides. In some embodiments, the one or more nucleotide modification is a deletion of 1, 2, 3, 4, 5, 6, 7, 8, 9, 10, 11, 12, 13, 14, 15, 16, 17, 18, 19, 20, 40, or 50 nucleotides. In some embodiments, the one or more nucleotide modification is a substitution of from about 1-10, 1-9, 1-8, 1-7, 1-6, 1-5, 1-4, 1-3, or 1-2 nucleotides. In some embodiments, the one or more nucleotide modification is a substitution of 1, 2, 3, 4, 5, 6, 7, 8, 9, or 10 nucleotides. In some embodiments, the one or more nucleotide modification is a one nucleotide substitution. In some embodiments, the one or more nucleotide modification is made in a gene associated with a disease. In some embodiments, the one or more nucleotide modification is made in a gene associated with an inherited disease.

In some embodiments, the method further comprises contacting the dsDNA polynucleotide with a ngRNA.

In some embodiments, the (i) the editing polypeptide's DNA binding nickase nicks strand of the target dsDNA polynucleotide generating a free single-stranded DNA (ssDNA) polynucleotide having a 3′ end; (ii) the ssDNA hybridizes to the primer binding site of the ttRNA; (iii) the reverse transcriptase reverse transcribes a strand of DNA from the 3′ end of said ssDNA using the reverse transcription template sequence as a template, thereby generating an extended sequence comprising a ssDNA flap that encodes the integration recognition sequence; and (iv) replacing the endogenous DNA strand immediately adjacent downstream of the nick on the target strand with the ssDNA flap, thereby installing the integration recognition sequence in the target dsDNA polynucleotide.

In some embodiments, the target dsDNA polynucleotide is within the genome of a cell.

In one aspect, provided herein are methods of site specifically integrating a polynucleotide of interest into a target dsDNA polynucleotide, the method comprising (1) incorporating an integration recognition sequence into a target location in the target dsDNA polynucleotide comprising contacting the target dsDNA polynucleotide with: the composition described herein or a system described herein; and (2) integrating the polynucleotide of interest into the target dsDNA polynucleotide, the method comprising contacting the target dsDNA polynucleotide with the polynucleotide of interest that comprises the polynucleotide of interest operably connected to a polynucleotide that comprises a sequence complementary or associated to the integration recognition sequence; wherein the integrase incorporates the polynucleotide of interest into the target dsDNA polynucleotide by integration, recombination, or reverse transcription of the sequence that is complementary or associated to the integration recognition sequence.

In one aspect, provided herein are methods of site-specifically integrating of a polynucleotide of interest into a target dsDNA polynucleotide in a cell, the method comprising: (1) incorporating an integration recognition sequence into a target location in a target dsDNA polynucleotide in a cell by introducing into a cell: (a)(i) an editing polypeptide comprising a DNA binding nickase (or a functional fragment or variant thereof), a reverse transcriptase (or a functional fragment or variant thereof), an aptamer binding protein (or a functional fragment or variant thereof), and an integrase (or a functional fragment or variant thereof), wherein each of the DNA binding nickase, reverse transcriptase, aptamer binding protein, and integrase are each operably connected in any order; or (a)(ii) a polynucleotide encoding the editing polypeptide of (a)(i); (b) a targeting gRNA comprising (i) a spacer and (ii) a scaffold; and (c) a ttRNA comprising (i) a primer binding site, (ii) a reverse transcription template sequence that comprises an integration sequence that comprises an integration recognition sequence, and (iii) an aptamer; wherein the editing polypeptide's DNA binding nickase nicks a strand of the target dsDNA polynucleotide, and the reverse transcriptase reverse transcribes the reverse transcription template sequence into an extended sequence that encodes the first integration recognition sequence or a complement thereof and the extended sequence is incorporated into the target location in the target dsDNA polynucleotide; and (2) integrating the polynucleotide of interest into the target dsDNA polynucleotide, the method comprising introducing into the cell: a polynucleotide that comprises the polynucleotide of interest operably connected to a polynucleotide that comprises a sequence complementary or associated to the integration recognition sequence; wherein the integrase incorporates the polynucleotide of interest into the target dsDNA polynucleotide by integration, recombination, or reverse transcription of the sequence that is complementary or associated to the integration recognition sequence, to thereby site-specifically integrate the polynucleotide of interest into the target dsDNA polynucleotide in a cell.

In some embodiments, step (a) comprises step (a)(i) introducing into the cell a polynucleotide encoding an editing polypeptide.

In some embodiments, the polynucleotide encoding a polypeptide is within a vector or particle. In some embodiments, the vector is a plasmid or viral vector.

In some embodiments, the particle is a nanoparticle (e.g., a lipid nanoparticle) or a viral particle.

In some embodiments, step (a) comprises step (a)(ii) introducing into the cell a polypeptide encoded by the polynucleotide of (a)(i).

In some embodiments, the polynucleotide encoding an editing polypeptide comprises a polynucleotide described herein, a vector described herein, a polypeptide described herein, a particle described herein, or a pharmaceutical composition described herein.

In some embodiments, the ttRNA comprises an RNA polynucleotide described herein, a DNA polynucleotide described herein, a vector described herein, a particle described herein, or a pharmaceutical composition described herein.

In some embodiments, the ttRNA is within a vector or particle. In some embodiments, the vector is a plasmid or viral vector.

In some embodiments, the particle is a nanoparticle (e.g., a lipid nanoparticle) or a viral particle.

In some embodiments, the aptamer binding protein is an MS2 coat protein (MCP), a Qβ coat protein, or a PP7 coat protein, or a functional fragment or variant thereof. In some embodiments, the aptamer binding protein is MCP or a functional fragment or variant thereof.

In some embodiments, each of the DNA binding nickase, reverse transcriptase, aptamer binding protein, and the integrase are each operably connected in any order each via a linker.

In some embodiments, each of the DNA binding nickase, reverse transcriptase, aptamer binding protein, and integrase are encoded in the following order from 5′ to 3′: the aptamer binding protein, the DNA binding nickase, the reverse transcriptase, and the integrase.

In some embodiments, the integrase is Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), or Minos, or any functional fragments or variants thereof.

In some embodiments, the DNA binding nickase is a Cas9-D10A, a Cas9-H840A, a Cas12a, or a Cas12b nickase, or a functional fragment or variant thereof.

In some embodiments, the reverse transcriptase is derived from a Moloney Murine Leukemia Virus (M-MLV) reverse transcriptase domain, transcription xenopolymerase (RTX), avian myeloblastosis virus reverse transcriptase (AMV-RT), or Eubacterium rectale maturase RT (MarathonRT).

In some embodiments, the aptamer is an MS2 aptamer, a Qβ aptamer, or a PP7 aptamer. In some embodiments, the aptamer is an MS2 aptamer.

In some embodiments, the ttRNA comprises an RNA polynucleotide described herein.

In some embodiments, the polynucleotide of interest comprises one or more nucleotide modification (e.g., insertion, deletion, or substitution) compared to the endogenous sequence of the target dsDNA polynucleotide. In some embodiments, the one or more nucleotide modification is an insertion of from about 1-50, 1-40, 1-30, 1-20, 1-10, 1-5, or 1-2 nucleotides. In some embodiments, the one or more nucleotide modification is an insertion of 1, 2, 3, 4, 5, 6, 7, 8, 9, 10, 11, 12, 13, 14, 15, 16, 17, 18, 19, 20, 30, 40, or 50 nucleotides. In some embodiments, the one or more nucleotide modification is deletion of from about 1-50, 1-1-30, 1-20, 1-10, 1-5, or 1-2 nucleotides. In some embodiments, the one or more nucleotide modification is a deletion of 1, 2, 3, 4, 5, 6, 7, 8, 9, 10, 11, 12, 13, 14, 15, 16, 17, 18, 19, 20, 40, or 50 nucleotides. In some embodiments, the one or more nucleotide modification is a substitution of from about 1-10, 1-9, 1-8, 1-7, 1-6, 1-5, 1-4, 1-3, or 1-2 nucleotides. In some embodiments, the one or more nucleotide modification is a substitution of 1, 2, 3, 4, 5, 6, 7, 8, 9, or 10 nucleotides. In some embodiments, the one or more nucleotide modification is a one nucleotide substitution. In some embodiments, the one or more nucleotide modification is made in a gene associated with a disease. In some embodiments, the one or more nucleotide modification is made in a gene associated with an inherited disease.

In some embodiments, the method further comprises contacting the dsDNA polynucleotide with a ngRNA.

In some embodiments, (i) the editing polypeptide's DNA binding nickase nicks strand of the target dsDNA polynucleotide generating a free single-stranded DNA (ssDNA) polynucleotide having a 3′ end; (ii) the ssDNA hybridizes to the primer binding site of the ttRNA; (iii) the reverse transcriptase reverse transcribes a strand of DNA from the 3′ end of said ssDNA using the reverse transcription template sequence as a template, thereby generating an extended sequence comprising a ssDNA flap that encodes the integration recognition sequence; and (iv) replacing the endogenous DNA strand immediately adjacent downstream of the nick on the target strand with the ssDNA flap, thereby installing the integration recognition sequence in the target dsDNA polynucleotide.

In some embodiments, the target dsDNA polynucleotide is within the genome of a cell.

In one aspect, provided herein are methods of site-specifically integrating of a polynucleotide of interest into a target dsDNA polynucleotide in a cell, the method comprising: (1) incorporating an integration recognition sequence into a target location in a target dsDNA polynucleotide in a cell by introducing into a cell: (a)(i) an editing polypeptide comprising a DNA binding nickase (or a functional fragment or variant thereof), a reverse transcriptase (or a functional fragment or variant thereof), and an aptamer binding protein (or a functional fragment or variant thereof), wherein each of the DNA binding nickase, reverse transcriptase, and aptamer binding protein, are each operably connected in any order; or (a)(ii) a polynucleotide encoding the editing polypeptide of (a)(i); (b) a targeting gRNA comprising (i) a spacer and (ii) a scaffold; and (c) a ttRNA comprising (i) a primer binding site, (ii) a reverse transcription template sequence that comprises an integration sequence that comprises an integration recognition sequence, and (iii) an aptamer; wherein the editing polypeptide's DNA binding nickase nicks a strand of the target dsDNA polynucleotide, and the reverse transcriptase reverse transcribes the reverse transcription template sequence into an extended sequence that encodes the first integration recognition sequence or a complement thereof and the extended sequence is incorporated into the target location in the target dsDNA polynucleotide; and (2) integrating the polynucleotide of interest into the target dsDNA polynucleotide, the method comprising introducing into the cell: an integrase (or a functional fragment or variant thereof) and a polynucleotide that comprises the polynucleotide of interest operably connected to a polynucleotide that comprises a sequence complementary or associated to the integration recognition sequence; wherein the integrase incorporates the polynucleotide of interest into the target dsDNA polynucleotide by integration, recombination, or reverse transcription of the sequence that is complementary or associated to the integration recognition sequence, to thereby site-specifically integrate the polynucleotide of interest into the target dsDNA polynucleotide in a cell.

In some embodiments, step (a) comprises step (a)(i) introducing into the cell a polynucleotide encoding an editing polypeptide.

In some embodiments, the polynucleotide encoding a polypeptide is within a vector or particle. In some embodiments, the vector is a plasmid or viral vector.

In some embodiments, the particle is a nanoparticle (e.g., a lipid nanoparticle) or a viral particle.

In some embodiments, step (a) comprises step (a)(ii) introducing into the cell a polypeptide encoded by the polynucleotide of (a)(i).

In some embodiments, the polynucleotide encoding an editing polypeptide comprises a polynucleotide described herein, a vector described herein, a polypeptide described herein, a particle described herein, or a pharmaceutical composition described herein.

In some embodiments, the ttRNA comprises an RNA polynucleotide described herein, a DNA polynucleotide described herein, a vector described herein, a particle described herein, or a pharmaceutical composition described herein.

In some embodiments, the ttRNA is within a vector or particle. In some embodiments, the vector is a plasmid or viral vector.

In some embodiments, the particle is a nanoparticle (e.g., a lipid nanoparticle) or a viral particle.

In some embodiments, the aptamer binding protein is an MS2 coat protein (MCP), a Qβ coat protein, or a PP7 coat protein, or a functional fragment or variant thereof. In some embodiments, the aptamer binding protein is MCP or a functional fragment or variant thereof.

In some embodiments, each of the DNA binding nickase, reverse transcriptase, aptamer binding protein, and the integrase are each operably connected in any order each via a linker.

In some embodiments, each of the DNA binding nickase, reverse transcriptase, aptamer binding protein, and integrase are encoded in the following order from 5′ to 3′: the aptamer binding protein, the DNA binding nickase, the reverse transcriptase, and the integrase.

In some embodiments, the integrase is Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), or Minos, or any functional fragments or variants thereof.

In some embodiments, the DNA binding nickase is a Cas9-D10A, a Cas9-H840A, a Cas12a, or a Cas12b nickase, or a functional fragment or variant thereof.

In some embodiments, the reverse transcriptase is derived from a Moloney Murine Leukemia Virus (M-MLV) reverse transcriptase domain, transcription xenopolymerase (RTX), avian myeloblastosis virus reverse transcriptase (AMV-RT), or Eubacterium rectale maturase RT (MarathonRT).

In some embodiments, the aptamer is an MS2 aptamer, a Qβ aptamer, or a PP7 aptamer. In some embodiments, the aptamer is an MS2 aptamer.

In some embodiments, the ttRNA comprises an RNA polynucleotide described herein.

In some embodiments, the polynucleotide of interest comprises one or more nucleotide modification (e.g., insertion, deletion or substitution) compared to the endogenous sequence of the target dsDNA polynucleotide. In some embodiments, the one or more nucleotide modification is an insertion of from about 1-50, 1-40, 1-30, 1-20, 1-10, 1-5, or 1-2 nucleotides. In some embodiments, the one or more nucleotide modification is an insertion of 1, 2, 3, 4, 5, 6, 7, 8, 9, 10, 11, 12, 13, 14, 15, 16, 17, 18, 19, 20, 30, 40, or 50 nucleotides. In some embodiments, the one or more nucleotide modification is deletion of from about 1-50, 1-1-30, 1-20, 1-10, 1-5, or 1-2 nucleotides. In some embodiments, the one or more nucleotide modification is a deletion of 1, 2, 3, 4, 5, 6, 7, 8, 9, 10, 11, 12, 13, 14, 15, 16, 17, 18, 19, 20, 40, or 50 nucleotides. In some embodiments, the one or more nucleotide modification is a substitution of from about 1-10, 1-9, 1-8, 1-7, 1-6, 1-5, 1-4, 1-3, or 1-2 nucleotides. In some embodiments, the one or more nucleotide modification is a substitution of 1, 2, 3, 4, 5, 6, 7, 8, 9, or 10 nucleotides. In some embodiments, the one or more nucleotide modification is a one nucleotide substitution. In some embodiments, the one or more nucleotide modification is made in a gene associated with a disease. In some embodiments, the one or more nucleotide modification is made in a gene associated with an inherited disease.

In some embodiments, the method further comprises contacting the dsDNA polynucleotide with a ngRNA.

In some embodiments, (i) the editing polypeptide's DNA binding nickase nicks strand of the target dsDNA polynucleotide generating a free single-stranded DNA (ssDNA) polynucleotide having a 3′ end; (ii) the ssDNA hybridizes to the primer binding site of the ttRNA; (iii) the reverse transcriptase reverse transcribes a strand of DNA from the 3′ end of said ssDNA using the reverse transcription template sequence as a template, thereby generating an extended sequence comprising a ssDNA flap that encodes the integration recognition sequence; and (iv) replacing the endogenous DNA strand immediately adjacent downstream of the nick on the target strand with the ssDNA flap, thereby installing the integration recognition sequence in the target dsDNA polynucleotide.

In some embodiments, the target dsDNA polynucleotide is within the genome of a cell.

In one aspect, provided herein are methods of site specifically integrating a polynucleotide of interest into a target dsDNA polynucleotide in a cell, the method comprising (1) incorporating an integration recognition sequence into a target location in the target dsDNA polynucleotide in a cell comprising introducing into the cell: a composition described herein or a system described herein; and (2) integrating the polynucleotide of interest into the target dsDNA polynucleotide, the method comprising: contacting the target dsDNA polynucleotide with the polynucleotide of interest that comprises the polynucleotide of interest operably connected to a polynucleotide that comprises a sequence complementary or associated to the integration recognition sequence; wherein the integrase incorporates the polynucleotide of interest into the target dsDNA polynucleotide by integration, recombination, or reverse transcription of the sequence that is complementary or associated to the integration recognition sequence.

In one aspect, provided herein are methods of treating a subject diagnosed with a disease associated with a genetic mutation, the method comprising administering (a) a composition described herein, or a system described herein to the subject; and (b) a polynucleotide that comprises a polynucleotide of interest operably connected to a polynucleotide that comprises a sequence complementary or associated to the integration recognition sequence.

In some embodiments, the polynucleotide of interest comprises a polynucleotide sequence that replaces and/or corrects an endogenous sequence in the genome of a cell of the subject that comprises the genetic mutation associated with disease.

### BRIEF DESCRIPTION OF THE DRAWINGS

FIG. 1 is a bar graph showing the percent AttB insertion in HEK293FT cells transfecting with a PASTE editing system and a ttRNA, showing integration recognition site insertion with trans-template RNA, as described in Example 1.

### DETAILED DESCRIPTION

PASTE editing utilizes a modified PRIME gene editing technique to site-specifically insert an integration site within a target polynucleotide (e.g., genome) and subsequently utilizing the site to integrate a polynucleotide of interest (See, e.g., U.S. Ser. No. 17/451,734, the entire contents of which are incorporated by reference herein for all purposes). PASTE-REPLACE editing utilizes PASTE but with a paired set of gRNAs that enable the simultaneous deletion of a polynucleotide sequence (e.g., a gene) and replacement of the polynucleotide with an exogenous polynucleotide of interest (e.g., a variant gene) (See, e.g., U.S. Ser. No. 17/451,734). The first step in PASTE and PASTE-REPLACE editing generally comprises the use of a nickase (e.g., a Cas9 nickase) fused to a reverse transcriptase and an extended gRNA (pegRNA). The pegRNA comprises at least three functional polynucleotides (i) a targeting sequence (targeting the nickase to the target polynucleotide site), (ii) a primer binding site (PBS), and (iii) a reverse transcriptase template sequence containing the integration site. However, providing all three of these functionalities in a single RNA molecule means the pegRNAs are relatively long (typically 150-200 nucleotides) making the pegRNA difficult and expensive to manufacture at a large scale, as would be required for therapeutic or diagnostic uses. Additionally, the long length of the pegRNAs may impact editing efficiency; for example, biochemical measurements show that the complex design of the pegRNA reduces its affinity to Cas9, and likely decreases the efficiency of the process. Here is has unexpectedly been found that the pegRNA can be split into two distinct RNA molecules while maintaining efficient PASTE editing. As such, the current disclosure provides improved PASTE editing systems that allow for efficient editing and enhanced manufacturability. Providing the PBS and the reverse transcriptase template sequence in a separate RNA molecule is particularly advantageous in technologies like PASTE and PASTE-REPLACE because they require the insertion of long (38-46 bp) integration sites (versus PRIME editing which in many instances requires only short reverse transcriptase template sequences encoding a single nucleotide change).

### 8.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The use of the singular forms herein includes the plural unless specifically stated otherwise. As used herein, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. Numeric ranges are inclusive of the numbers defining the range.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

When proteins are contemplated herein, it should be understood that polynucleotides encoding the proteins are also provided, as are vectors comprising the polynucleotides encoding the proteins.

As used herein, the term “aptamer” refers to a single stranded RNA (ssRNA) polynucleotide that under suitable conditions forms a secondary structure comprising a hairpin loop that is specifically recognized (i.e., bound by) by a cognate protein (i.e., an aptamer binding protein). Examples of aptamers include, MS2, Qβ, and PP7.

As used herein, the term “aptamer binding protein” refers to a protein that specifically recognizes (i.e., binds to) an aptamer. Examples of aptamer binding proteins that specifically bind cognate aptamers are the MS2 coat protein (MCP) (which binds the MS2 aptamer), Qβ coat protein (which binds the Qβ aptamer), and the PP7 coat protein (which binds the PP7 aptamer).

As used herein, the term “Cas9” refers to an RNA-guided nuclease comprising a Cas9 domain, or a fragment thereof (e.g., a protein comprising an active or inactive DNA cleavage domain of Cas9, and/or the gRNA binding domain of Cas9). The amino acid sequence of an exemplary Cas9 reference polypeptide is provided in SEQ ID NO: 1.

As used herein, the term “Cas9 nickase” refers to a variant of Cas9 which is capable of cleaving only one strand of a target double stranded polynucleotide, thereby introducing a single-strand break in the target double strand polynucleotide. Similar terminology is used herein in reference to other Cas nucleases that exhibit nickase activity. For example, a “Cas12e nickase” would be used similarly herein to refer to a Cas12e which is capable of cleaving only one strand of a target double stranded polynucleotide, thereby introducing a single-strand break in the target double strand polynucleotide

As used herein, the term “derived from,” with reference to a polynucleotide sequence refers to a polynucleotide sequence that has at least 85%, 90%, 95%, 96%, 97%, 98%, or 99% sequence identity to a reference naturally occurring nucleic acid sequence from which it is derived. The term “derived from,” with reference to an amino acid sequence refers to an amino acid sequence that has at least 85%, 90%, 95%, 96%, 97%, 98%, or 99% sequence identity to a reference naturally occurring amino acid sequence from which it is derived. The term “derived from” as used herein does not denote any specific process or method for obtaining the polynucleotide or amino acid sequence. For example, the polynucleotide or amino acid sequence can be chemically synthesized.

As used herein, the term “diagnostic nucleotide modification” refers to a polynucleotide of interest that encodes at least one nucleotide modification (e.g., substitution, deletion, or insertion) relative to the endogenous target polynucleotide (e.g., gene) sequence, wherein the polynucleotide of interest is not intended to have or does not have a therapeutic effect in a subject but is intended to be used in diagnostic methods.

As used herein, the term “DNA” or “DNA polynucleotides” refers to macromolecules that include multiple deoxyribonucleotides that are polymerized via phosphodiester bonds. Deoxyribonucleotides are nucleotides in which the sugar is deoxyribose.

As used herein, the term “editing polypeptide” refers to a multifunctional protein comprising (i) a DNA binding nickase (or functional fragment or variant thereof) (e.g., as described herein), (ii) a reverse transcriptase (or functional fragment or variant thereof) (e.g., as described herein), and (iii) an aptamer binding protein (or a functional fragment or variant thereof) (e.g., as described herein). In some embodiments, the editing polypeptide further comprises (iv) an integrase (or a functional fragment or variant thereof) (e.g., as described herein).

As used herein, the term “editing polynucleotide” refers to a polynucleotide encoding an editing polypeptide (e.g., described herein).

As used herein, the term “functional fragment” in reference to a protein refers to a fragment of a reference protein that retains at least one particular function. For example, a functional fragment of an aptamer binding protein can refer to a fragment of the protein that retains the ability to bind the cognate aptamer. Not all functions of the reference protein need be retained by a functional fragment of the protein. In some instances, one or more functions are selectively reduced or eliminated.

As used herein, the term “functional variant” in reference to a protein refers to a protein that comprises at least one amino acid modification (e.g., a substitution, deletion, addition) compared to the amino acid sequence of a reference protein, that retains at least one particular function. For example, a functional variant of an aptamer binding protein refers to a protein that binds an aptamer comprising an amino acid substitution as compared to a wild type reference protein that retains the ability to bind the cognate aptamer. Not all functions of the reference wild type protein need be retained by the functional variant of the protein. In some instances, one or more functions are selectively reduced or eliminated.

As used herein, the term “fusion protein” and grammatical equivalents thereof refer to a protein that comprises an amino acid sequence derived from at least two separate proteins. The amino acid sequence of the at least two separate proteins can be directly connected through a peptide bond; or can be operably connected through an amino acid linker. Therefore, the term fusion protein encompasses embodiments, wherein the amino acid sequence of e.g., Protein A is directly connected to the amino acid sequence of Protein B through a peptide bond (Protein A-Protein B), and embodiments, wherein the amino acid sequence of e.g., Protein A is operably connected to the amino acid sequence of Protein B through an amino acid linker (Protein A-linker-Protein B).

A used herein, the term “fuse” and grammatical equivalents thereof refer to the operable connection of an amino acid sequence derived from one protein to the amino acid sequence derived from different protein. The term fuse encompasses both a direct connection of the two amino acid sequences through a peptide bond, and the indirect connection through an amino acid linker.

As used herein, the term “guide RNA” or “gRNA” refers to an RNA polynucleotide that guides the insertion or deletion of one or more polynucleotides of interest (e.g., a gene of interest) into a target polynucleotide (e.g., genome) via a nuclease (e.g., a Cas protein, e.g., Cas9).

As used herein, the term “integrase” refers to a protein capable of integrating a polynucleotide of interest (e.g., a gene) into a desired location (e.g., at an integration site) in a target polynucleotide (e.g., the genome of a cell). The integration can occur in a single reaction or multiple reactions.

As used herein, the term “integration sequence” refers to a polynucleotide sequence that encodes an integration site.

As used herein, the term “integration site” refers to a polynucleotide sequence capable of being recognized by an integrase.

As used herein, the term “modification,” with reference to a polynucleotide sequence, refers to a polynucleotide sequence that comprises at least one substitution, alteration, inversion, addition, or deletion of nucleotide compared to a reference polynucleotide sequence. Modifications can include the inclusion of non-naturally occurring nucleotide residues. As used herein, the term “modification,” with reference to an amino acid sequence refers to an amino acid sequence that comprises at least one substitution, alteration, inversion, addition, or deletion of an amino acid residue compared to a reference amino acid sequence. Modifications can include the inclusion of non-naturally occurring amino acid residues. Naturally occurring amino acid derivatives are not considered modified amino acids for purposes of determining percent identity of two amino acid sequences. For example, a naturally occurring modification of a glutamate amino acid residue to a pyroglutamate amino acid residue would not be considered an amino acid modification for purposes of determining percent identity of two amino acid sequences. Further, for example, a naturally occurring modification of a glutamate amino acid residue to a pyroglutamate amino acid residue would not be considered an amino acid “modification” as defined herein.

As used herein, the term “nickase” refers to a protein (e.g., a nuclease) that has the ability to cleave only one strand of a target double stranded polynucleotide, thereby introducing a single-strand break in the target double strand polynucleotide. In some embodiments, for example, an editing polypeptide described herein comprises a Cas9 nuclease with one of the two nuclease domains inactivated, e.g., by amino acid substitution of H840A, wherein the Cas9 has nickase activity but is not able to make a double strand break in a target double stranded polynucleotide.

As used herein, the term “nicking gRNA” refers to a targeting gRNA that targets a nickase to the strand of a double stranded DNA target polynucleotide that was not edited during an engineering method (e.g., PRIME, PASTE, or PASTE-REPLACE) (e.g., before DNA repair of the heteroduplex comprising an edited strand and a non-edited strand).

In reference to a system utilizing a first targeting gRNA, a nickase, and a ttRNA, refers to a second targeting gRNA that is utilized to target the nickase (e.g., a Cas9 nickase) to the opposite strand of a double stranded DNA polynucleotide that was targeted by the first targeting RNA.

As used herein, the terms “operably connected” and “operably linked” are used interchangeably and refer to a linkage of polynucleotide sequence elements or polypeptide sequence elements in a functional relationship. For example, a polynucleotide sequence is operably connected when it is placed into a functional relationship with another polynucleotide sequence. In some embodiments, a transcription regulatory polynucleotide sequence e.g., a promoter, enhancer, or other expression control element is operably-linked to a polynucleotide sequence that encodes a protein if it affects the transcription of the polynucleotide sequence that encodes the protein.

As used herein, the term “orthogonal integration sites” refers to integrations sites that do not significantly recognize the recognition site or nucleotide sequence of the integrase (e.g., recombinase) recognized by the other.

The determination of “percent identity” between two sequences (e.g., polypeptide or polynucleotides) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the)(BLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (See, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance for use in contact with the tissues of human beings and/or non-human animals without excessive toxicity, irritation, allergic response, or other problem or complication, commensurate with a reasonable therapeutic benefit/risk ratio.

The terms “polynucleotide,” “nucleic acid,” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “polynucleotide of interest” refers to a polynucleotide intended or desired to be integrated into a target polynucleotide using any suitable method (e.g., a method described herein).

As used herein, the term “primer binding site” or “PBS” refers to the portion of a ttRNA that binds to the polynucleotides sequence ay the 3′ end of the flap that is formed after the DNA binding nickase nicks the target polynucleotide sequence.

The terms “protein” and “polypeptide” are used interchangeably herein and refer to a polymer of at least two amino acids linked by a peptide bond.

As used herein, the term “protospacer” refers to the DNA sequence that has the same (or similar) nucleotide sequence as the spacer sequence of a gRNA. The gRNA anneals to the complement of the protospacer sequence on the opposite strand of the DNA.

As used herein, the term “protospacer adjacent motif” or “PAM” refers to a short DNA sequence, typically 2-6 base pairs, that functions to aid a Cas nickase in recognizing the target DNA.

As used herein, the term “recognition site” refers to a polynucleotide sequence that pairs with an integration site to mediate integration by an integrase (e.g., a recombinase).

As used herein, the term “RNA” or “RNA polynucleotide” refers to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

As used herein, the term “hairpin loop” in reference to an RNA polynucleotide (e.g., an aptamer) refers to an RNA sequence that under physiological conditions is able to base-pair to form a double helix that ends in an unpaired loop.

As used herein, the term “reverse transcriptase” refers to a protein (e.g., a polymerase) that is capable of RNA-dependent DNA synthesis. All known reverse transcriptases require a primer to synthesize a DNA transcript from an RNA template. An exemplary reverse transcriptase commonly used in the art is derived from the moloney murine leukemia virus (M-MLV). See, e.g., Gerard, G. R., DNA 5:271-279 (1986) and Kotewicz, M. L., et al., Gene 35:249-258 (1985).

As used herein, the term “reverse transcriptase template sequence” refers to the portion of a ttRNA that encodes the polynucleotide desired to be integrated into the target polynucleotide (e.g., genome) that is synthesized by the reverse transcriptase. The reverse transcriptase template sequence is used as a template during DNA synthesis by the reverse transcriptase. The reverse transcription template sequence can be reverse transcribed by the reverse transcriptase into an extended sequence that encodes an integration recognition sequence or a complement thereof.

As used herein, the term “scaffold” in reference to a gRNA refers to a polynucleotide in a gRNA that mediates binding to a nuclease (e.g., nickase) or a functional fragment or variant thereof (e.g., Cas9 (e.g., Cas9 nickases)).

As used herein, the term “spacer” in reference to a gRNA refers to a polynucleotide in a gRNA that mediates binding to a polynucleotide comprising a sequence complementary to the protospacer.

As used herein, the term “trans-template RNA” or “ttRNA” refers to an RNA polynucleotide that comprises at least a primer binding site and a reverse transcriptase template sequence. In some embodiments, the ttRNA further comprises one or more aptamers that is specifically recognized by a cognate aptamer binding protein.

As used herein, the term “targeting gRNA” refers to a gRNA that comprises a spacer and a scaffold. The targeting gRNA functions to guide an editing polypeptide (e.g., an editing polypeptide described herein, see, e.g., § 5.3) to the target polynucleotide (e.g., a specific target sequence within a genome, e.g., within a cell).

As used herein, the term “therapeutic nucleotide modification” refers to a polynucleotide of interest that encodes at least one nucleotide modification (e.g., substitution, deletion, or insertion) relative to the endogenous target polynucleotide (e.g., gene) sequence that is intended to have or does have a therapeutic effect in a subject.

A “therapeutically effective amount” of a therapeutic agent (e.g., a composition or system described herein) refers to any amount of the therapeutic agent that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a compositions as described herein.

### 8.2 PRIME, PASTE, and PASTE-REPLACE Editing

In some embodiments, the compositions and systems described herein are useful in methods of PRIME, PASTE (programmable addition via site-specific targeting elements), and PASTE-REPLACE editing. PRIME editing generally involves the use of Cas9 nickase fused to a reverse-transcriptase and an extended gRNA (pegRNA). The pegRNA comprises a standard guide sequence (e.g., a spacer and a scaffold to target the Cas9 to the target site), a PBS) and a reverse transcriptase template sequence containing the desired nucleotide edit (see, e.g., Scholefield, J., Harrison, P. T. Prime editing—an update on the field. Gene Ther 28, 396-401 (2021). https://doi.org/10.1038/s41434-021-00263-9). PASTE editing utilizes a modified PRIME technique to site-specifically insert an integration site within a target polynucleotide and subsequently utilizing the site to integrate a polynucleotide sequence of interest (see, e.g., US20220145293, the entire contents of which are incorporated by reference herein for all purposes). PASTE-REPLACE editing utilizes PASTE but with a paired set of guides that enable the simultaneous deletion of a target polynucleotide sequence (e.g., a gene) and replacement of the target polynucleotide with an exogenous polynucleotide of interest (e.g., a genre).

### 8.3 Editing Polypeptides

In one aspect, provided herein are editing polypeptides and methods of using the editing polypeptides in site-specific polynucleotide (e.g., gene, genome) engineering. The editing polypeptide is a multifunctional fusion protein, responsible for e.g., nicking the target polynucleotide, reverse transcribing the reverse transcriptase template sequence of a ttRNA (e.g., described herein), and recruiting a ttRNA (e.g., described herein) through binding to an aptamer within the ttRNA. In some embodiments, the editing polypeptide further comprises integrase functionality (e.g., for use in PASTE and PASTE-REPLACE methods of polynucleotide editing).

Also provided herein are polynucleotides encoding any of the editing polypeptides described herein. In some embodiments, the polynucleotides encoding the editing polypeptides are RNA or DNA. Also provided herein are vectors comprising a polynucleotide encoding any of the editing polynucleotides described herein. In some embodiments, the vector is a plasmid or a viral vector. Also provided herein are particles comprising the editing polypeptide, the polynucleotide encoding the editing polypeptide, or the vector comprising the polynucleotide encoding the editing polypeptide. In some embodiments, the particle is a viral particle or a lipid particle (e.g., a lipid nanoparticle). Also provided herein are cells comprising the editing polypeptide, polynucleotide encoding the editing polypeptide, the vector comprising the polynucleotide encoding the editing polypeptide, or the particle comprising the editing polypeptide, the polynucleotide encoding the editing polypeptide, or the vector comprising the polynucleotide encoding the editing polypeptide. Also provided herein are pharmaceutical compositions comprising the editing polypeptide, polynucleotide encoding the editing polypeptide, the vector comprising the polynucleotide encoding the editing polypeptide, or the particle comprising the editing polypeptide, the polynucleotide encoding the editing polypeptide, or the vector comprising the polynucleotide encoding the editing polypeptide; and a pharmaceutically acceptable excipient. Also provided herein are kits comprising the editing polypeptide, polynucleotide encoding the editing polypeptide, the vector comprising the polynucleotide encoding the editing polypeptide, or the particle comprising the editing polypeptide, the polynucleotide encoding the editing polypeptide, or the vector comprising the polynucleotide encoding the editing polypeptide; and a pharmaceutically acceptable excipient, or a pharmaceutical composition comprising any of the foregoing and a pharmaceutically acceptable excipient.

Each of the above referenced components of editing polypeptides (and polynucleotides) is further described below.

### 8.3.1 DNA Binding Nickases

As outlined above, one component of the editing polypeptides described herein is a DNA binding nickase (or a functional fragment or variant thereof). In some embodiments, a functional fragment or a functional variant of a DNA binding nickase is used, wherein the fragment or variant maintains nickase activity.

In some embodiments, the DNA binding nickase is a naturally occurring nickase (or functional fragment or variant thereof). In some embodiments, the DNA binding nickase (or a functional fragment or variant thereof) is a nickase that has been modified (e.g., incorporates one or more amino acid modifications compared to a reference sequence) to impart nickase activity. For example, the DNA binding nickase (or a functional fragment or variant thereof) may be a Cas9 nuclease (or functional fragment or variant thereof) with one of the two nuclease domains inactivated, e.g., by amino acid substitution of H840A, wherein the Cas9 has nickase activity but is not able to make a double strand break in a target double stranded polynucleotide.

In some embodiments, the DNA binding nickase comprises a Cas9 nickase, Cas12e (CasX) nickase, Cas12d (CasY) nickase, Cas12a (Cpf1) nickase, Cas12b1 (C2c1) nickase, Cas13a (C2c2) nickase, Cas12c (C2c3) nickase (or a functional fragment or variant of any of the foregoing).

In some embodiments, the DNA binding nickase is a Cas9 nickase (or a functional fragment or variant thereof). The wild type Cas9 comprises two separate nuclease domains, the RuvC domain (which cleaves the non-protospacer DNA strand) and HNH domain (which cleaves the protospacer DNA strand). In some embodiments, the Cas9 nickase comprises only a single functioning nuclease domain.

In some embodiments, the Cas9 nickase comprises a mutation in the RuvC domain which inactivates the RuvC nuclease activity. Suitable mutations include, but are not limited to, e.g., in aspartate (D) 10, histidine (H) 983, aspartate (D) 986, or glutamate (E) 762 (amino acid numbering relative to SEQ ID NO: 1), (See, e.g., Nishimasu et al., “Crystal structure of Cas9 in complex with guide RNA and target DNA,” Cell/156(5), 935-949, which is incorporated herein by reference). In some embodiments, the Cas9 nickase (or a functional fragment or variant thereof) comprises at least one of the following amino acid substitutions D10X, H983X, D986X, or E762X, wherein Xis any amino acid other than the wild-type amino acid (amino acid numbering relative to SEQ ID NO: 1). In some embodiments, the Cas9 nickase (or a functional fragment or variant thereof) comprises at least one of the following amino acid substitutions D10A, H983A, D986A, or E762A, or a combination thereof (amino acid numbering relative to SEQ ID NO: 1). A Cas9 nickase (or a functional fragment or variant thereof) comprising a D10A amino acid substitution is also referred to herein as Cas9-D10A. Likewise, a Cas9 nickase (or a functional fragment or variant thereof) comprising a H983A amino acid substitution is also referred to herein as Cas9-H983A. A Cas9 nickase (or a functional fragment or variant thereof) comprising a D986A amino acid substitution is also referred to herein as Cas9-D986A. A Cas9 nickase (or a functional fragment or variant thereof) comprising a E762A amino acid substitution is also referred to herein as Cas9-E762A.

In some embodiments, the Cas9 nickase (or a functional fragment or variant thereof) comprises a mutation in the HNH domain which inactivates the HNH nuclease activity. Suitable mutations include, but are not limited to, a mutation in histidine (H) 840 or asparagine (R) 863 (amino acid numbering relative to SEQ ID NO: 1) (See supra). In some embodiments, the Cas9 nickase (or a functional fragment or variant thereof) comprises at least one of the following amino acid substitutions H840X or R863X, wherein X is any amino acid other than the wild-type amino acid (amino acid numbering relative to SEQ ID NO: 1). In some embodiments, the Cas9 nickase (or a functional fragment or variant thereof) comprises at least one of the following amino acid substitutions H840A or R863A, or a combination thereof (amino acid numbering relative to SEQ ID NO: 1). A Cas9 nickase (or a functional fragment or variant thereof) comprising an H840A amino acid substitution is also referred to herein as Cas9-H840A. Likewise, a Cas9 nickase (or a functional fragment or variant thereof) comprising an R863A amino acid substitution is also referred to herein as a Cas9-R863A (amino acid numbering relative to SEQ ID NO: 1).

In some embodiments, the DNA binding nickase (or a functional fragment or variant thereof) comprises Cas9-D10A, Cas9-H983A, Cas9-D986A, Cas9-E762A, Ca9s-H840A, or Cas9-R863A (or a functional fragment or variant of any of the foregoing). In some embodiments, the DNA binding nickase (or a functional fragment or variant thereof) comprises Cas9-D10A, Cas9-H983A, Cas9-D986A, or Cas9-E762A (or a functional fragment or variant of any of the foregoing). In some embodiments, the DNA binding nickase comprises Cas9-H840A or Cas9-R863A (or a functional fragment or variant of any of the foregoing). In some embodiments, the DNA binding nickase (or a functional fragment or variant thereof) comprises Cas9-H840A (or a functional fragment or variant of any of the foregoing).

The amino acid sequence of exemplary DNA binding nickase is provided in Table 1.

TABLE 1
The amino acid sequence of exemplary DNA binding nickase.

| Description | Amino Acid Sequence | SEQ ID NO |
|---|---|---|
| Cas9 | MDKKYSIGLDIGTNSVGWAVITDEYKVPSKKFKVLGNTDRH | 1 |
| Reference | SIKKNLIGALLFDSGETAEATRLKRTARRRYTRRKNRICYL | |
| (Wild-Type) | QEIFSNEMAKVDDSFFHRLEESELVEEDKKHERHPIFGNIV | |
| | DEVAYHEKYPTIYHLRKKLVDSTDKADLRLIYLALAHMIKE | |
| | RGHFLIEGDLNPDNSDVDKLFIQLVQTYNQLFEENPINASG | |
| | VDAKAILSARLSKSRRLENLIAQLPGEKKNGLFGNLIALSL | |
| | GLTPNFKSNFDLAEDAKLQLSKDTYDDDLDNLLAQIGDQYA | |
| | DLFLAAKNLSDAILLSDILRVNTEITKAPLSASMIKRYDEH | |
| | HQDLTLLKALVRQQLPEKYKEIFFDQSKNGYAGYIDGGASQ | |
| | EEFYKFIKPILEKMDGTEELLVKLNREDLLRKQRTFDNGSI | |
| | PHQIHLGELHAILRRQEDFYPFLKDNREKIEKILTFRIPYY | |
| | VGPLARGNSRFAWMTRKSEETITPWNFEEVVDKGASAQSFI | |
| | ERMTNFDKNLPNEKVLPKHSLLYEYFTVYNELTKVKYVTEG | |
| | MRKPAFLSGEQKKAIVDLLFKTNRKVTVKQLKEDYFKKIEC | |
| | FDSVEISGVEDRFNASLGTYHDLLKIIKDKDELDNEENEDI | |
| | LEDIVLTLTLFEDREMIEERLKTYAHLFDDKVMKQLKRRRY | |
| | TGWGRLSRKLINGIRDKQSGKTILDFLKSDGFANRNEMQLI | |
| | HDDSLTFKEDIQKAQVSGQGDSLHEHIANLAGSPAIKKGIL | |
| | QTVKVVDELVKVMGRHKPENIVIEMARENQTTQKGQKNSRE | |
| | RMKRIEEGIKELGSQILKEHPVENTQLQNEKLYLYYLQNGR | |
| | DMYVDQELDINRLSDYDVDHIVPQSFLKDDSIDNKVLTRSD | |
| | KNRGKSDNVPSEEVVKKMKNYWRQLLNAKLITQRKFDNLTK | |
| | AERGGLSELDKAGFIKRQLVETRQITKHVAQILDSRMNTKY | |
| | DENDKLIREVKVITLKSKLVSDFRKDFQFYKVREINNYHHA | |
| | HDAYLNAVVGTALIKKYPKLESEFVYGDYKVYDVRKMIAKS | |
| | EQEIGKATAKYFFYSNIMNFFKTEITLANGEIRKRPLIETN | |
| | GETGEIVWDKGRDFATVRKVLSMPQVNIVKKTEVQTGGFSK | |
| | ESILPKRNSDKLIARKKDWDPKKYGGFDSPTVAYSVLVVAK | |
| | VEKGKSKKLKSVKELLGITIMERSSFEKNPIDFLEAKGYKE | |
| | VKKDLIIKLPKYSLFELENGRKRMLASAGELQKGNELALPS | |
| | KYVNFLYLASHYEKLKGSPEDNEQKQLFVEQHKHYLDEIIE | |
| | QISEFSKRVILADANLDKVLSAYNKHRDKPIREQAENIIHL | |
| | FTLTNLGAPAAFKYFDTTIDRKRYTSTKEVLDATLIHQSIT | |
| | GLYETRIDLSQLGGD | |
| Cas9-D10A | MDKKYSIGLAIGTNSVGWAVITDEYKVPSKKFKVLGNT | 2 |
| | DRHSIKKNLIGALLFDSGETAEATRLKRTARRRYTRRK | |
| | NRICYLQEIFSNEMAKVDDSFFHRLEESFLVEEDKKHE | |
| | RHPIFGNIVDEVAYHEKYPTIYHLRKKLVDSTDKADLR | |
| | LIYLALAHMIKFRGHFLIEGDLNPDNSDVDKLFIQLVQ | |
| | TYNQLFEENPINASGVDAKAILSARLSKSRRLENLIAQ | |
| | LPGEKKNGLFGNLIALSLGLTPNEKSNFDLAEDAKLQL | |
| | SKDTYDDDLDNLLAQIGDQYADLFLAAKNLSDAILLSD | |
| | ILRVNTEITKAPLSASMIKRYDEHHQDLTLLKALVRQQ | |
| | LPEKYKEIFFDQSKNGYAGYIDGGASQEEFYKFIKPIL | |
| | EKMDGTEELLVKLNREDLLRKQRTFDNGSIPHQIHLGE | |
| | LHAILRRQEDFYPFLKDNREKIEKILTFRIPYYVGPLA | |
| | RGNSRFAWMTRKSEETITPWNFEEVVDKGASAQSFIER | |
| | MTNFDKNLPNEKVLPKHSLLYEYFTVYNELTKVKYVTE | |
| | GMRKPAFLSGEQKKAIVDLLFKTNRKVTVKQLKEDYFK | |
| | KIECFDSVEISGVEDRENASLGTYHDLLKIIKDKDELD | |
| | NEENEDILEDIVLTLTLFEDREMIEERLKTYAHLEDDK | |
| | VMKQLKRRRYTGWGRLSRKLINGIRDKQSGKTILDELK | |
| | SDGFANRNFMQLIHDDSLTFKEDIQKAQVSGQGDSLHE | |
| | HIANLAGSPAIKKGILQTVKVVDELVKVMGRHKPENIV | |
| | IEMARENQTTQKGQKNSRERMKRIEEGIKELGSQILKE | |
| | HPVENTQLQNEKLYLYYLQNGRDMYVDQELDINRLSDY | |
| | DVDHIVPQSFLKDDSIDNKVLTRSDKNRGKSDNVPSEE | |
| | VVKKMKNYWRQLLNAKLITQRKFDNLTKAERGGLSELD | |
| | KAGFIKRQLVETRQITKHVAQILDSRMNTKYDENDKLI | |
| | REVKVITLKSKLVSDFRKDFQFYKVREINNYHHAHDAY | |
| | LNAVVGTALIKKYPKLESEFVYGDYKVYDVRKMIAKSE | |
| | QEIGKATAKYFFYSNIMNFFKTEITLANGEIRKRPLIE | |
| | TNGETGEIVWDKGRDFATVRKVLSMPQVNIVKKTEVQT | |
| | GGFSKESILPKRNSDKLIARKKDWDPKKYGGFDSPTVA | |
| | YSVLVVAKVEKGKSKKLKSVKELLGITIMERSSFEKNP | |
| | IDFLEAKGYKEVKKDLIIKLPKYSLFELENGRKRMLAS | |
| | AGELQKGNELALPSKYVNFLYLASHYEKLKGSPEDNEQ | |
| | KQLFVEQHKHYLDEIIEQISEFSKRVILADANLDKVLS | |
| | AYNKHRDKPIREQAENIIHLFTLTNLGAPAAFKYEDTT | |
| | IDRKRYTSTKEVLDATLIHQSITGLYETRIDLSQLGGD | |
| Cas9-H840A | MDKKYSIGLDIGTNSVGWAVITDEYKVPSKKFKVLGNT | 3 |
| | DRHSIKKNLIGALLFDSGETAEATRLKRTARRRYTRRK | |
| | NRICYLQEIFSNEMAKVDDSFFHRLEESFLVEEDKKHE | |
| | RHPIFGNIVDEVAYHEKYPTIYHLRKKLVDSTDKADLR | |
| | LIYLALAHMIKFRGHFLIEGDLNPDNSDVDKLFIQLVQ | |
| | TYNQLFEENPINASGVDAKAILSARLSKSRRLENLIAQ | |
| | LPGEKKNGLFGNLIALSLGLTPNEKSNEDLAEDAKLQL | |
| | SKDTYDDDLDNLLAQIGDQYADLFLAAKNLSDAILLSD | |
| | ILRVNTEITKAPLSASMIKRYDEHHQDLTLLKALVRQQ | |
| | LPEKYKEIFFDQSKNGYAGYIDGGASQEEFYKFIKPIL | |
| | EKMDGTEELLVKLNREDLLRKQRTEDNGSIPHQIHLGE | |
| | LHAILRRQEDFYPFLKDNREKIEKILTFRIPYYVGPLA | |
| | RGNSRFAWMTRKSEETITPWNFEEVVDKGASAQSFIER | |
| | MTNFDKNLPNEKVLPKHSLLYEYFTVYNELTKVKYVTE | |
| | GMRKPAFLSGEQKKAIVDLLFKTNRKVTVKQLKEDYFK | |
| | KIECFDSVEISGVEDRFNASLGTYHDLLKIIKDKDELD | |
| | NEENEDILEDIVLTLTLFEDREMIEERLKTYAHLEDDK | |
| | VMKQLKRRRYTGWGRLSRKLINGIRDKQSGKTILDELK | |
| | SDGFANRNFMQLIHDDSLTFKEDIQKAQVSGQGDSLHE | |
| | HIANLAGSPAIKKGILQTVKVVDELVKVMGRHKPENIV | |
| | IEMARENQTTQKGQKNSRERMKRIEEGIKELGSQILKE | |
| | HPVENTQLQNEKLYLYYLQNGRDMYVDQELDINRLSDY | |
| | DVDAIVPQSFLKDDSIDNKVLTRSDKNRGKSDNVPSEE | |
| | VVKKMKNYWRQLLNAKLITQRKFDNLTKAERGGLSELD | |
| | KAGFIKRQLVETRQITKHVAQILDSRMNTKYDENDKLI | |
| | REVKVITLKSKLVSDFRKDFQFYKVREINNYHHAHDAY | |
| | LNAVVGTALIKKYPKLESEFVYGDYKVYDVRKMIAKSE | |
| | QEIGKATAKYFFYSNIMNFFKTEITLANGEIRKRPLIE | |
| | TNGETGEIVWDKGRDFATVRKVLSMPQVNIVKKTEVQT | |
| | GGFSKESILPKRNSDKLIARKKDWDPKKYGGFDSPTVA | |
| | YSVLVVAKVEKGKSKKLKSVKELLGITIMERSSFEKNP | |
| | IDFLEAKGYKEVKKDLIIKLPKYSLFELENGRKRMLAS | |
| | AGELQKGNELALPSKYVNFLYLASHYEKLKGSPEDNEQ | |
| | KQLFVEQHKHYLDEIIEQISEFSKRVILADANLDKVLS | |
| | AYNKHRDKPIREQAENIIHLFTLTNLGAPAAFKYFDTT | |
| | IDRKRYTSTKEVLDATLIHQSITGLYETRIDLSQLGGD | |

In some embodiments, the DNA binding nickase comprises a nickase set forth in Table 1 (or a functional fragment or variant thereof). In some embodiments, the DNA binding nickase comprises an amino acid sequence at least 95%, 96%, 97%, 98%, 99%, or 100% identical to the amino acid sequence of a DNA binding nickase set forth in Table 1. In some embodiments, the DNA binding nickase comprises an amino acid sequence comprising the amino acid sequence of any one of the DNA binding nickases set forth in Table 1, and comprising 1, 2, or 3 amino acid modifications. In some embodiments, the DNA binding nickase comprises an amino acid sequence comprising the amino acid sequence of any one of the DNA binding nickases set forth in Table 1, and comprises no more than 1, 2, or 3 amino acid modifications.

In some embodiments, the DNA binding nickase comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-3. In some embodiments, the DNA binding nickase comprises an amino acid sequence comprising the amino acid sequence of any one of SEQ ID NOS: 1-3, and comprising 1, 2, or 3 amino acid modifications. In some embodiments, the DNA binding nickase comprises an amino acid sequence comprising the amino acid sequence of any one of SEQ ID NOS: 1-3, and comprises no more than 1, 2, or 3 amino acid modifications.

### 8.3.2 Reverse Transcriptases

As outlined above, one component of the editing polypeptides described herein is a reverse transcriptase (or a functional fragment or variant thereof). In some embodiments, a functional fragment or functional variants of a reverse transcriptase is used, wherein the fragment or variant maintains reverse transcriptase activity.

In some embodiments, the reverse transcriptase is a naturally occurring reverse transcriptase (or functional fragment or variant thereof). In some embodiments, the reverse transcriptase is derived from a naturally occurring reverse transcriptase (or functional fragment or variant thereof). In some embodiments, the reverse transcriptase (or a functional fragment or variant thereof) is a reverse transcriptase that has been modified (e.g., incorporates one or more amino acid modifications compared to a reference sequence). In some embodiments, the modified reverse transcriptase comprises one or more improved properties as compared to the corresponding reference sequence (e.g., thermostability, fidelity, reverse transcriptase activity).

Exemplary reverse transcriptases include, but are not limited to, moloney murine leukemia virus (M-MLV) reverse transcriptase; human immunodeficiency virus (HIV) reverse transcriptase and avian sarcoma-leukosis virus (ASLV) reverse transcriptase, which includes but is not limited to rous sarcoma virus (RSV) reverse transcriptase, avian myeloblastosis virus (AMY) reverse transcriptase, avian erythroblastosis virus (AEV) helper virus MCAV reverse transcriptase, avian myelocytomatosis virus MC29 helper virus MCAV reverse transcriptase, avian reticuloendotheliosis virus (REV-T) helper virus REV-A reverse transcriptase, avian sarcoma virus UR2 helper virus UR2AV reverse transcriptase, avian sarcoma virus Y73 helper virus YAV reverse transcriptase, rous associated virus (RAV) reverse transcriptase, and myeloblastosis associated virus (MAV) reverse transcriptase.

Any of the forementioned exemplary reverse transcriptases can be modified, e.g., comprises at least one amino acid substitution, deletion, or addition.

In some embodiments, the reverse transcriptase is derived from the M-MLV reverse transcriptase. The amino acid sequence of an exemplary reference M-MLV reverse transcriptase is set forth in SEQ ID NO: 5. In some embodiments, the M-MLV reverse transcriptase is naturally occurring. In some embodiments, the M-MLV reverse transcriptase is non-naturally occurring. In some embodiments, the M-MLV reverse transcriptase comprises one or more amino acid modifications relative to a reference sequence (e.g., SEQ ID NO: 5). In some embodiments, the M-MLV reverse transcriptase comprises one or more amino acid substitutions relative to a reference sequence (e.g., SEQ ID NO: 5).

In some embodiments, the M-MLV reverse transcriptase comprises 1, 2, 3, 4, 5, 6, 7, 8, 9, 10, or more of the following amino acid substitutions: Y8H, P51L, S56A, S67R, E69K, V129P, L139P, T197A, D200N, H204R, V223H, T246E, N249D, E286R, Q291I, E302K, E302R, T306K, F309N, W313F, M320L, T330P, P330E, L435G, L435R, N454K, D524A, D524G, D524N, E562Q, D583N, H594Q, L603W, E607K, D653N, or L671P, or any combination thereof (amino acid numbering relative to SEQ ID NO: 5). In some embodiments, the M-MLV reverse transcriptase comprises 1, 2, 3, 4, or 5 of the following amino acid substitutions: D200N, T306K, W313F, T330P, or L603W, or any combination thereof (amino acid numbering relative to SEQ ID NO:).

In some embodiments, the reverse transcriptase is fused (either directly or indirectly via a linker) of the DNA-binding protein Sso7d from a species of Sulfurisphaera. The amino acid sequence of an exemplary Sso7d DNA binding domain is provided in Table 2.

TABLE 2
The amino acid sequence of exemplary Sso7d DNA binding domain.

| Description | Amino Acid Sequence | SEQ ID NO |
|---|---|---|
| Sso7d DNA | ALLLDTDRVQFGPVVALNPATLLPLPEEGLQHNCLDGTGGG | 4 |
| binding | GVTVKFKYKGEELEVDISKIKKVWRVGKMISFTYDDNGKTG | |
| domain | RGAVSEKDAPKELLQMLEKSGKKSGGSKRTADGS | |

The amino acid sequence of exemplary reverse transcriptases is provided in Table 3.

TABLE 3
The amino acid sequence of exemplary reverse transcriptases.

| Description | Amino Acid Sequence | SEQ ID NO |
|---|---|---|
| M-MLV | TLNIEDEYRLHETSKEPDVSLGSTWLSDFPQAWAETGGMGL | 5 |
| Reverse | AVRQAPLIIPLKATSTPVSIKQYPMSQEARLGIKPHIQRLL | |
| Transcriptase | DQGILVPCQSPWNTPLLPVKKPGTNDYRPVQDLREVNKRVE | |
| Reference | DIHPTVPNPYNLLSGLPPSHQWYTVLDLKDAFFCLRLHPTS | |
| (Wild-Type) | QPLFAFEWRDPEMGISGQLTWTRLPQGFKNSPTLFDEALHR | |
| | DLADFRIQHPDLILLQYVDDLLLAATSELDCQQGTRALLQT | |
| | LGNLGYRASAKKAQICQKQVKYLGYLLKEGQRWLTEARKET | |
| | VMGQPTPKTPRQLREFLGTAGFCRLWIPGFAEMAAPLYPLT | |
| | KTGTLFNWGPDQQKAYQEIKQALLTAPALGLPDLTKPFELF | |
| | VDEKQGYAKGVLTQKLGPWRRPVAYLSKKLDPVAAGWPPCL | |
| | RMVAAIAVLTKDAGKLTMGQPLVILAPHAVEALVKQPPDRW | |
| | LSNARMTHYQALLLDTDRVQFGPVVALNPATLLPLPEEGLQ | |
| | HNCLDILAEAHGTRPDLTDQPLPDADHTWYTDGSSLLQEGQ | |
| | RKAGAAVTTETEVIWAKALPAGTSAQRAELIALTQALKMAE | |
| | GKKLNVYTDSRYAFATAHIHGEIYRRRGLLTSEGKEIKNKD | |
| | EILALLKALFLPKRLSIIHCPGHQKGHSAEARGNRMADQAA | |
| | RKAAITETPDTSTLLIENSSP | |
| M-MLV | TLNIEDEYRLHETSKEPDVSLGSTWLSDFPQAWAETGGMGL | 6 |
| Reverse | AVRQAPLIIPLKATSTPVSIKQYPMSQEARLGIKPHIQRLL | |
| Transcriptase | DQGILVPCQSPWNTPLLPVKKPGTNDYRPVQDLREVNKRVE | |
| Reference | DIHPTVPNPYNLLSGLPPSHQWYTVLDLKDAFFCLRLHPTS | |
| (Wild-Type- | QPLFAFEWRDPEMGISGQLTWTRLPQGFKNSPTLFDEALHR | |
| C-terminal | DLADFRIQHPDLILLQYVDDLLLAATSELDCQQGTRALLQT | |
| truncated) | LGNLGYRASAKKAQICQKQVKYLGYLLKEGQRWLTEARKET | |
| | VMGQPTPKTPRQLREFLGTAGFCRLWIPGFAEMAAPLYPLT | |
| | KTGTLFNWGPDQQKAYQEIKQALLTAPALGLPDLTKPFELF | |
| | VDEKQGYAKGVLTQKLGPWRRPVAYLSKKLDPVAAGWPPCL | |
| | RMVAAIAVLTKDAGKLTMGQPLVILAPHAVEALVKQPPDRW | |
| | LSNARMTHYQALLLDTDRVQFGPVVALNPATLLPLPEEGLQ | |
| | HNCLD | |
| M-MLV | TLNIEDEYRLHETSKEPDVSLGSTWLSDFPQAWAETGGMGL | 7 |
| Reverse | AVRQAPLIIPLKATSTPVSIKQYPMSQEARLGIKPHIQRLL | |
| Transcriptase | DQGILVPCQSPWNTPLLPVKKPGTNDYRPVQDLREVNKRVE | |
| D200N/ | DIHPTVPNPYNLLSGLPPSHQWYTVLDLKDAFFCLRLHPTS | |
| T306K/T330P/ | QPLFAFEWRDPEMGISGQLTWTRLPQGFKNSPTLFNEALHR | |
| L603W/ | DLADFRIQHPDLILLQYVDDLLLAATSELDCQQGTRALLQT | |
| W313F | LGNLGYRASAKKAQICQKQVKYLGYLLKEGQRWLTEARKET | |
| | VMGQPTPKTPRQLREFLGKAGFCRLFIPGFAEMAAPLYPLT | |
| | KPGTLFNWGPDQQKAYQEIKQALLTAPALGLPDLTKPFELF | |
| | VDEKQGYAKGVLTQKLGPWRRPVAYLSKKLDPVAAGWPPCL | |
| | RMVAAIAVLTKDAGKLTMGQPLVILAPHAVEALVKQPPDRW | |
| | LSNARMTHYQALLLDTDRVQFGPVVALNPATLLPLPEEGLQ | |
| | HNCLDILAEAHGTRPDLTDQPLPDADHTWYTDGSSLLQEGQ | |
| | RKAGAAVTTETEVIWAKALPAGTSAQRAELIALTQALKMAE | |
| | GKKLNVYTDSRYAFATAHIHGEIYRRRGWLTSEGKEIKNKD | |
| | EILALLKALFLPKRLSIIHCPGHQKGHSAEARGNRMADQAA | |
| | RKAAITETPDTSTLLIENSSP | |
| M-MLV | TLNIEDEYRLHETSKEPDVSLGSTWLSDFPQAWAETGGMGL | 8 |
| Reverse | AVRQAPLIIPLKATSTPVSIKQYPMSQEARLGIKPHIQRLL | |
| Transcriptase | DQGILVPCQSPWNTPLLPVKKPGTNDYRPVQDLREVNKRVE | |
| D200N/ | DIHPTVPNPYNLLSGLPPSHQWYTVLDLKDAFFCLRLHPTS | |
| T306K/T330P/ | QPLFAFEWRDPEMGISGQLTWTRLPQGFKNSPTLFNEALHR | |
| L603W/ | DLADFRIQHPDLILLQYVDDLLLAATSELDCQQGTRALLQT | |
| W313F | LGNLGYRASAKKAQICQKQVKYLGYLLKEGQRWLTEARKET | |
| (Truncated) | VMGQPTPKTPRQLREFLGKAGFCRLFIPGFAEMAAPLYPLT | |
| | KPGTLFNWGPDQQKAYQEIKQALLTAPALGLPDLTKPFELF | |
| | VDEKQGYAKGVLTQKLGPWRRPVAYLSKKLDPVAAGWPPCL | |
| | RMVAAIAVLTKDAGKLTMGQPLVILAPHAVEALVKQPPDRW | |
| | LSNARMTHYQALLLDTDRVQFGPVVALNPATLLPLPEEGLQ | |
| | HNCLD | |

In some embodiments, the reverse transcriptase comprises a reverse transcriptase set forth in Table 2 (or a functional fragment or variant thereof). In some embodiments, the reverse transcriptase comprises an amino acid sequence at least 95%, 96%, 97%, 98%, 99%, or 100% identical to the amino acid sequence of a reverse transcriptase set forth in Table 2. In some embodiments, the reverse transcriptase comprises an amino acid sequence comprising the amino acid sequence of any one of the reverse transcriptases set forth in Table 2, and comprising 1, 2, or 3 amino acid modifications. In some embodiments, the reverse transcriptase comprises an amino acid sequence comprising the amino acid sequence of any one of the reverse transcriptases set forth in Table 2, and comprises no more than 1, 2, or 3 amino acid modifications.

In some embodiments, the reverse transcriptase comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 5-8. In some embodiments, the reverse transcriptase comprises an amino acid sequence comprising the amino acid sequence of any one of SEQ ID NOS: 5-8, and comprising 1, 2, or 3 amino acid modifications. In some embodiments, the reverse transcriptase comprises an amino acid sequence comprising the amino acid sequence of any one of SEQ ID NOS: 5-8, and comprises no more than 1, 2, or 3 amino acid modifications.

### 8.3.3 Aptamer Binding Proteins

As described above, the polynucleotide editing systems (e.g., gene editing systems) described herein utilize an aptamer and aptamer binding protein pair; wherein the ttRNA comprises an aptamer and the editing polypeptide comprises an aptamer binding protein (or a functional fragment or functional variant thereof) that specifically recognizes (i.e., binds to) the aptamer of the ttRNA. The use of the aptamer/aptamer binding protein pair allows for recruitment of the ttRNA directly to the location on the polynucleotide that is to be targeted by the editing system through the binding of the aptamer to the aptamer binding protein (that is itself targeted by fusion to the DNA binding nickase (e.g., described herein) which is in turn targeted to the target location on the polynucleotide through binding to a gRNA comprising the spacer and a scaffold (e.g., as described herein). Any suitable aptamer/aptamer binding protein pair known to the person of ordinary skill in the art may be employed. Exemplary pairs include, but are not limited to, an MS2 RNA aptamer/MS2 coat protein (MCP) pair, a Qβ RNA aptamer/Qβ coat protein pair, and a PP7 RNA aptamer/PP7 coat protein. The aptamer binding proteins are further detailed below, and the corresponding aptamers are further detailed below.

Exemplary aptamer binding proteins include, but are not limited to, MCP, Qβ coat protein, and PP7 coat protein. As discussed above, MCP specifically recognizes (i.e., binds to) to the MS2 aptamer; the Qβ coat protein specifically recognizes (i.e., binds to) to the Qβ aptamer; and the PP7 coat protein specifically recognizes (i.e., binds to) to the aptamer. The amino acid sequence of exemplary aptamer binding proteins is provided in Table 4.

TABLE 4
Amino acid sequence of exemplary aptamer binding proteins.

| Description | Amino Acid Sequence | SEQ ID NO |
|---|---|---|
| MCP (N55K) | MASNFTQFVLVDNGGTGDVTVAPSNFANGVAEWISS | 9 |
| | NSRSQAYKVTCSVRQSSAQKRKYTIKVEVPKVATQT | |
| | VGGVELPVAAWRSYLNMELTIPIFATNSDCELIVKA | |
| | MQGLLKDGNPIPSAIAANSGIYSA | |
| Qβ Coat | MAKLETVTLGNIGKDGKQTLVLNPRGVNPTNGVASL | 10 |
| Protein | SQAGAVPALEKRVTVSVSQPSRNRKNYKVQVKIQNP | |
| | TACTANGSCDPSVTRQAYADVTFSFTQYSTDEERAF | |
| | VRTELAALLASPLLIDAIDQLNPAY | |
| PP7 Coat | SKTIVLSVGEATRTLTEIQSTADRQIFEEKVGPLVG | 11 |
| Protein | RLRLTASLRQNGAKTAYRVNLKLDQADVVDCSTSVC | |
| | GELPKVRYTQVWSHDVTIVANSTEASRKSLYDLTKS | |
| | LVVQATSEDLVVNLVPLGR | |

In some embodiments, the full amino acid sequence of the mature form of the aptamer binding protein is utilizes. Functional variants or fragments of the aptamer binding proteins (compared to a reference sequence) may be utilized, as long as the fragments or variants are capable of specifically recognizing (i.e., binding) the cognate aptamer.

In some embodiments, the aptamer binding protein comprises an aptamer binding protein set forth in Table 4. In some embodiments, the aptamer binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of an aptamer binding protein set forth in Table 4. In some embodiments, the aptamer binding protein comprises an amino acid sequence comprising the amino acid sequence of any one of the aptamer binding proteins set forth in Table 4, and comprising 1, 2, or 3 amino acid modifications. In some embodiments, the aptamer binding protein comprises an amino acid sequence comprising the amino acid sequence of any one of the aptamer binding proteins set forth in Table 4, and comprises no more than 1, 2, or 3 amino acid modifications.

In some embodiments, the aptamer binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 9-11. In some embodiments, the aptamer binding protein comprises an amino acid sequence comprising the amino acid sequence of any one of SEQ ID NOS: 9-11, and comprising 1, 2, or 3 amino acid modifications. In some embodiments, the aptamer protein comprises an amino acid sequence comprising the amino acid sequence of any one of SEQ ID NOS: 9-11, and comprises no more than 1, 2, or 3 amino acid modifications.

### 8.3.4 Integrases

In some embodiments, the compositions, systems, and methods described herein utilize an integrase (or a functional fragment or variant thereof) and a cognate integration sequence (e.g., comprising an integration site, see, e.g., § 5.4.1.2). Integrases, integration sequences, and integration sites are particularly useful in methods of PASTE editing (e.g., as described herein). It is understood by the person of ordinary skill in the art that integration sites and integrases for use in the compositions, systems, and methods described herein will be selected in pairs, wherein the selected integrase will specifically recognize the selected integration site.

The integrase (or functional fragment or variant thereof) can be provided as part of the editing polypeptide (e.g., as described herein, e.g., as a fusion protein) or as a separate polypeptide. In some embodiments, the integrase (or functional fragment or variant thereof) is part of the editing polypeptide (e.g., a fusion protein). In some embodiments, the integrase (or functional fragment or variant thereof) is polypeptide separate from the editing polypeptide.

Exemplary integrases include recombinases, reverse transcriptases, and retrotransposases. Exemplary integrases include, but are not limited to, Cre, Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, and retrotransposases encoded by R2, L1, Tol2 Tc1, Tc3, Mariner (Himar 1), Mariner (mos 1), and Minos. In some embodiments, the integrase is Bxb1.

The integrases (e.g., recombinases) explicitly provided herein are not meant to be exclusive examples of integrases (e.g., recombinases) that can be used in embodiments of the disclosure. The methods and compositions of the disclosure can be expanded by mining databases for new orthogonal integrases (e.g., recombinases) or designing synthetic integrases (e.g., recombinases) with defined DNA specificities (See, e.g., Groth et al., “Phage integrases: biology and applications.” J. Mol. Biol. 2004; 335, 667-678; Gordley et al., “Synthesis of programmable integrases.” Proc. Natl. Acad. Sci. USA. 2009; 106, 5053-5058; the entire contents of each of which is hereby incorporated by reference in their entirety for all purposes).

In some embodiments, the integrase (or functional fragment or variant thereof) is a recombinase that incorporates the polynucleotide of interest into the target polynucleotide (e.g., a genome of a cell) at an integration site by recombination. Exemplary recombinases include serine recombinases and tyrosine recombinases. In some embodiments, the integrase is a serine recombinase. In some embodiments, the integrase is a tyrosine recombinase. Exemplary serine recombinases include, but are not limited to, Hin, Gin, Tn3, β-six, CinH, ParA, γδ, Bxb1, φC31, TP901, TG1, φBT1, R1, R2, R3, R4, R5, φRV1, φFC1, MR11, A118, U153, gp29. Examples of serine recombinases also include, without limitation, recombinases Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, and BxZ2 from Mycobacterial phages. In some embodiments, the integrase is Hin, Gin, Tn3, β-six, CinH, ParA, γδ, Bxb1, φC31, TP901, TG1, φBT1, R1, R2, R3, R4, R5, φRV1, φFC1, MR11, A118, U153, or gp29. In some embodiments, the integrase is a tyrosine recombinase. Exemplary, tyrosine recombinases include, but are not limited to, Cre, FLP, R, Lambda, HK101, HK022, and pSAM2.

In some embodiments, the integrase is a reverse transcriptase that incorporates the polynucleotide of interest into the target polynucleotide (e.g., a genome of a cell) at an integration site by reverse transcription.

In some embodiments, the integrase (or functional fragment or variant thereof) is a retrotransposase that incorporates the polynucleotide of interest into the target polynucleotide (e.g., a genome of a cell) at an integration site by retrotransposition. Exemplary retrotransposases include, but are not limited to, retrotransposases encoded by elements such as R2, L1, Tol2 Tc1, Tc3, Mariner (Himar 1), Mariner (mos 1), Minos, and any functional variants thereof.

The amino acid sequence of exemplary integrases is provided in Table 5.

TABLE 5
The amino acid sequence of exemplary integrases.

| Description | Amino Acid Sequence | SEQ ID NO |
|---|---|---|
| Bxb1 Integrase | SRALVVIRLSRVTDATTSPERQLESCQQLCAQRGWD | 12 |
| | VVGVAEDLDVSGAVDPFDRKRRPNLARWLAFEEQPF | |
| | DVIVAYRVDRLTRSIRHLQQLVHWAEDHKKLVVSAT | |
| | EAHFDTTTPFAAVVIALMGTVAQMELEAIKERNRSA | |
| | AHFNIRAGKYRGSLPPWGYLPTRVDGEWRLVPDPVQ | |
| | RERILEVYHRVVDNHEPLHLVAHDLNRRGVLSPKDY | |
| | FAQLQGREPQGREWSATALKRSMISEAMLGYATLNG | |
| | KTVRDDDGAPLVRAEPILTREQLEALRAELVKTSRA | |
| | KPAVSTPSLLLRVLFCAVCGEPAYKFAGGGRKHPRY | |
| | RCRSMGFPKHCGNGTVAMAEWDAFCEEQVLDLLGDA | |
| | ERLEKVWVAGSDSAVELAEVNAELVDLTSLIGSPAY | |
| | RAGSPQREALDARIAALAARQEELEGLEARPSGWEW | |
| | RETGQRFGDWWREQDTAAKNTWLRSMNVRLTFDVRG | |
| | GLTRTIDFGDLQEYEQHLRLGSVVERLHTGMS | |

In some embodiments, the full amino acid sequence of an integrase is utilized. Functional variants or fragments of an integrase e (compared to a reference sequence) may be utilized, as long as the fragments or variants are capable of mediating integration.

In some embodiments, the integrase comprises an integrase set forth in Table 5. In some embodiments, the integrase comprises an amino acid sequence at least 85%, 86%, 87%, 88%, 89%, 90%, 91%, 92%, 93%, 94%, 95%, 96%, 97%, 98%, 99%, or 100% identical to the amino acid sequence of an integrase set forth in Table 5. In some embodiments, the integrase comprises an amino acid sequence comprising the amino acid sequence of any one of the integrases set forth in Table 5, and comprising 1, 2, or 3 amino acid modifications. In some embodiments, the integrase comprises an amino acid sequence comprising the amino acid sequence of any one of the integrases set forth in Table 5, and comprises no more than 1, 2, or 3 amino acid modifications.

In some embodiments, the integrase comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 12. In some embodiments, the integrase comprises an amino acid sequence comprising the amino acid sequence of SEQ ID NO: 12, and comprising 1, 2, or 3 amino acid modifications. In some embodiments, the integrase comprises an amino acid sequence comprising the amino acid sequence of SEQ ID NO: 12, and comprises no more than 1, 2, or 3 amino acid modifications.

### 8.3.5 Linkers

As described above, any one or more (e.g., all) of the components of an editing polypeptide (e.g., described herein) can be operably connected via a linker (e.g., a peptide linker) (e.g., one or more different linkers). Common linkers (e.g., glycine and glycine/serine linkers) are known in the art. Any suitable linker(s) can be utilized as long as each component can mediate the desired function.

In some embodiments, at least two components of an editing polypeptide (e.g., described herein) are operably connected via a linker. In some embodiments, each component of an editing polypeptide (e.g., described herein) is operably connected to the preceding and/or subsequent component of the editing polypeptide via a linker. In some embodiments, each component of an editing polypeptide (e.g., described herein) is operably connected to the preceding and/or subsequent component of the editing polypeptide via a different linker.

In some embodiments, the linker is from about 2-100, 2-50, 2-25, 2-10, 4-100, 4-50, 4-25, 4-10, 5-100, 5-50, 5-25, 5-10, 10-100, 10-50, or 10-25 amino acids in length. In some embodiments, the linker is about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 amino acids in length.

The amino acid sequence of exemplary linkers is provided in Table 6.

TABLE 6
The amino acid sequence of exemplary linkers.

| Description | Amino Acid Sequence | SEQ ID NO |
|---|---|---|
| GGGGS | GGGGS | 13 |
| (GGGS)₃ | GGGGSGGGGSGGGGS | 14 |
| (GGS)₆ | GGSGGSGGSGGSGGSGGS | 15 |
| PAPAP | PAPAP | 16 |
| (EAAAK)₃ | EAAAKEAAAKEAAAK | 17 |
| XTEN | SGSETPGTSESATPES | 18 |
| EAAAK | EAAAK | 19 |

In some embodiments, the editing polypeptide comprises one or more of the linkers set forth in Table 6. In some embodiments, the editing polypeptide comprises a plurality of the linkers set forth in Table 6. In some embodiments, the editing polypeptide comprises at least one linker comprising an amino acid sequence of one of the linkers in Table 6, comprising 1, 2, or 3 amino acid modifications (e.g., substitutions). In some embodiments, the editing polypeptide comprises at least one linker comprising an amino acid sequence of one of the linkers in Table 6, comprising no more than 1, 2, or 3 amino acid modifications (e.g., substitutions). In some embodiments, the editing polypeptide comprises a linker comprising amino acid sequence at least 95%, 96%, 97%, 98%, 99%, or 100% identical to the amino acid sequence of any one of SEQ ID NOS: 13-19. In some embodiments, the editing polypeptide comprises a linker that comprises the amino acid sequence of SEQ ID NOS: 13-19, and comprises 1, 2, or 3 amino acid modifications.

### 8.3.6 Nuclear Localization Signal (NLS)

In some embodiments, the editing polypeptide comprises a nuclear localization signal (NLS), which promotes translocation of the editing polypeptide to the nucleus within a host cell. The editing polypeptide may contain more than one NLS. NLS are known to the person of ordinary skill in the art. Any suitable NLS known in the art may be employed. The amino acid sequence of exemplary NLSs is provided in Table 7.

TABLE 7
Amino acid sequence of exemplary NLSs.

| | SEQ |
|---|---|
| Amino Acid Sequence | ID NO |
| AHFKISGEKRPSTDPGKKAKNPKKKKKKDP | 20 |
| AHRAKKMSKTHA | 21 |
| ASPEYVNLPINGNG | 22 |
| CTKRPRW | 23 |
| DKAKRVSRNKSEKKRR | 24 |
| EELRLKEELLKGIYA | 25 |
| EEQLRRRKNSRLNNTG | 26 |
| EVLKVIRTGKRKKKAWKRMVTKVC | 27 |
| HHHHHHHHHHHHQPH | 28 |
| HKKKHPDASVNFSEFSK | 29 |
| HKRTKKNLS | 30 |
| IINGRKLKLKKSRRRSSQTSNNSFTSRRS | 31 |
| KAEQERRK | 32 |
| KEKRKRREELFIEQKKRK | 33 |
| KKGKDEWFSRGKKP | 34 |
| KKGPSVQKRKKTNLS | 35 |
| KKKTVINDLLHYKKEK | 36 |
| KKNGGKGKNKPSAKIKK | 37 |
| KKPKWDDFKKKKK | 38 |
| KKRKKDNLS | 39 |
| KKRRKRRRK | 40 |
| KKRRRRARK | 41 |
| KKSKRGR | 42 |
| KKSRKRGS | 43 |
| KKSTALSRELGKIMRRR | 44 |
| KKSYQDPEIIAHSRPRK | 45 |
| KKTGKNRKLKSKRVKTR | 46 |
| KKVSIAGQSGKLWRWKR | 47 |
| KKYENVVIKRSPRKRGRPRK | 48 |
| KNKKRK | 49 |
| KPKKKR | 50 |
| KRAMKDDSHGNSTSPKRRK | 51 |
| KRANSNLVAAYEKAKKK | 52 |
| KRASEDTTSGSPPKKSSAGPKR | 53 |
| KRFKRRWMVRKMKTKK | 54 |
| KRGLNSSFETSPKKVK | 55 |
| KRGNSSIGPNDLSKRKQRKK | 56 |
| KRIHSVSLSQSQIDPSKKVKRAK | 57 |
| KRKGKLKNKGSKRKK | 58 |
| KRRRRRRREKRKR | 59 |
| KRSNDRTYSPEEEKQRRA | 60 |
| KRTVATNGDASGAHRAKKMSK | 61 |
| KRVYNKGEDEQEHLPKGKKR | 62 |
| KSGKAPRRRAVSMDNSNK | 63 |
| KVNFLDMSLDDIIIYKELE | 64 |
| KVQHRIAKKTTRRRR | 65 |
| LSPSLSPL | 66 |
| MDSLLMNRRKFLYQFKNVRWAKGRRETYLC | 67 |
| MPQNEYIELHRKRYGYRLDYHEKKRKKESREAHERSKKAKKMI | 68 |
| GLKAKLYHK | |
| MVQLRPRASR | 69 |
| NNKLLAKRRKGGASPKDDPMDDIK | 70 |
| NYKRPMDGTYGPPAKRHEGE | 71 |
| PDTKRAKLDSSETTMVKKK | 72 |
| PEKRTKI | 73 |
| PGGRGKKK | 74 |
| PGKMDKGEHRQERRDRPY | 75 |
| PKKGDKYDKTD | 76 |
| PKKKSRK | 77 |
| PKKNKPE | 78 |
| PKKRAKV | 79 |
| PKPKKLKVE | 80 |
| PKRGRGR | 81 |
| PKRRLVDDA | 82 |
| PKRRRTY | 83 |
| PLEKRR | 84 |
| PLRKAKR | 85 |
| PPAKRKCIF | 86 |
| PPARRRRL | 87 |
| PPKKKRKV | 88 |
| PPNKRMKVKH | 89 |
| PPRIYPQLPSAPT | 90 |
| PQRSPFPKSSVKR | 91 |
| PRPRKVPR | 92 |
| PRRRVQRKR | 93 |
| PRRVRLK | 94 |
| PSRKRPR | 95 |
| PSSKKRKV | 96 |
| PTKKRVK | 97 |
| QRPGPYDRP | 98 |
| RGKGGKGLGKGGAKRHRK | 99 |
| RKAGKGGGGHKTTKKRSAKDEKVP | 100 |
| RKIKLKRAK | 101 |
| RKIKRKRAK | 102 |
| RKKEAPGPREELRSRGR | 103 |
| RKKRKGK | 104 |
| RKKRRQRRR | 105 |
| RKKSIPLSIKNLKRKHKRKKNKITR | 106 |
| RKLVKPKNTKMKTKLRTNPY | 107 |
| RKRLILSDKGQLDWKK | 108 |
| RKRLKSK | 109 |
| RKRRVRDNM | 110 |
| RKRSPKDKKEKDLDGAGKRRKT | 111 |
| RKRTPRVDGQTGENDMNKRRRK | 112 |
| RLPVRRRRRR | 113 |
| RLRFRKPKSK | 114 |
| RQQRKR | 115 |
| RRDLNSSFETSPKKVK | 116 |
| RRDRAKLR | 117 |
| RRGDGRRR | 118 |
| RRGRKRKAEKQ | 119 |
| RRKKRR | 120 |
| RRKRSKSEDMDSVESKRRR | 121 |
| RRKRSR | 122 |
| RRPKGKTLQKRKPK | 123 |
| RRRGFERFGPDNMGRKRK | 124 |
| RRRGKNKVAAQNCRK | 125 |
| RRRKRRNLS | 126 |
| RRRQKQKGGASRRR | 127 |
| RRRREGPRARRRR | 128 |
| RRTIRLKLVYDKCDRSCKIQKKNRNKCQYCRFHKCLSVGMSHN | 129 |
| AIRFGRMPRSEKAKLKAE | |
| RRVPQRKEVSRCRKCRK | 130 |
| RVGGRRQAVECIEDLLNEPGQPLDLSCKRPRP | 131 |
| RVVKLRIAP | 132 |
| RVVRRR | 133 |
| SKRKTKISRKTR | 134 |
| SYVKTVPNRTRTYIKL | 135 |
| TGKNEAKKRKIA | 136 |
| TLSPASSPSSVSCPVIPASTDESPGSALNI | 137 |

In some embodiments, the editing polypeptide comprises one or more of the NLSs set forth in Table 7. In some embodiments, the editing polypeptide comprises a plurality of the NSLs set forth in Table 7. In some embodiments, the editing polypeptide comprises at least one NLS comprising an amino acid sequence of one of the linkers in Table 7, comprising 1, 2, or 3 amino acid modifications (e.g., substitutions). In some embodiments, the editing polypeptide comprises at least one linker comprising an amino acid sequence of one of the NLSs in Table 7, comprising no more than 1, 2, or 3 amino acid modifications (e.g., substitutions). In some embodiments, the editing polypeptide comprises an NLS comprising amino acid sequence at least 95%, 96%, 97%, 98%, 99%, or 100% identical to the amino acid sequence of any one of SEQ ID NOS: 20-137. In some embodiments, the editing polypeptide comprises an NLS that comprises the amino acid sequence of SEQ ID NOS: 20-137, and comprises 1, 2, or 3 amino acid modifications.

### 8.3.7 Orientation

As described above, the editing polypeptides described herein are fusion proteins comprising (i) a DNA binding nickase DNA binding nickase (or functional fragment or variant thereof) (e.g., as described herein), (ii) a reverse transcriptase (or functional fragment or variant thereof) (e.g., as described herein), and (iii) an aptamer binding protein (or a functional fragment or variant thereof) (e.g., as described herein). In some embodiments, the editing polypeptide further comprises (iv) an integrase (or a functional fragment or variant thereof) (e.g., as described herein).

The components of the editing polypeptide can be arranged in any order in which each component can mediate the desired function. In some embodiments, the editing polypeptide comprises from N- to C-terminus an aptamer binding protein (or a functional fragment or variant thereof) (e.g., as described herein); a DNA binding nickase (or functional fragment or variant thereof) (e.g., as described herein); and a reverse transcriptase (or functional fragment or variant thereof) (e.g., as described herein). In some embodiments, the editing polypeptide comprises from N- to C-terminus an aptamer binding protein (or a functional fragment or variant thereof) (e.g., as described herein); a DNA binding nickase (or functional fragment or variant thereof) (e.g., as described herein), a reverse transcriptase (or functional fragment or variant thereof) (e.g., as described herein); and (iv) an integrase (or a functional fragment or variant thereof) (e.g., as described herein).

In some embodiments, each component is operably connected to the subsequent and/or following component through a peptide linker (e.g., described herein). In some embodiments, the editing polypeptide comprises from N- to C-terminus an aptamer binding protein (or a functional fragment or variant thereof) (e.g., as described herein); a first peptide linker; a DNA binding nickase (or functional fragment or variant thereof) (e.g., as described herein); a second peptide linker; and a reverse transcriptase (or functional fragment or variant thereof) (e.g., as described herein). In some embodiments, the editing polypeptide comprises from N- to C-terminus an aptamer binding protein (or a functional fragment or variant thereof) (e.g., as described herein); a first peptide linker; a DNA binding nickase (or functional fragment or variant thereof) (e.g., as described herein); a second peptide linker; a reverse transcriptase (or functional fragment or variant thereof) (e.g., as described herein); a third peptide linker; and (iv) an integrase (or a functional fragment or variant thereof) (e.g., as described herein).

Likewise, each component of an editing polynucleotide encoding an editing poly described herein can be arranged in any order in which each component can mediate the desired function. In some embodiments, the editing polynucleotide comprises from 5′- to 3′-terminus a polynucleotide encoding an aptamer binding protein (or a functional fragment or variant thereof) (e.g., as described herein); a polynucleotide encoding a DNA binding nickase (or functional fragment or variant thereof) (e.g., as described herein); and a polynucleotide encoding a reverse transcriptase (or functional fragment or variant thereof) (e.g., as described herein). In some embodiments, the editing polynucleotide comprises from 5′- to 3′-terminus a polynucleotide encoding an aptamer binding protein (or a functional fragment or variant thereof) (e.g., as described herein); a polynucleotide encoding a DNA binding nickase (or functional fragment or variant thereof) (e.g., as described herein); a polynucleotide encoding a reverse transcriptase (or functional fragment or variant thereof) (e.g., as described herein); and a polynucleotide encoding an integrase (or a functional fragment or variant thereof) (e.g., as described herein).

In some embodiments, the editing polynucleotide comprises from 5′- to 3′-terminus a polynucleotide encoding an aptamer binding protein (or a functional fragment or variant thereof) (e.g., as described herein); a first linker; a polynucleotide encoding a DNA binding nickase (or functional fragment or variant thereof) (e.g., as described herein); a second linker; and a polynucleotide encoding a reverse transcriptase (or functional fragment or variant thereof) (e.g., as described herein). In some embodiments, the editing polynucleotide comprises from 5′- to 3′-terminus a polynucleotide encoding an aptamer binding protein (or a functional fragment or variant thereof) (e.g., as described herein); a first linker; a polynucleotide encoding a DNA binding nickase (or functional fragment or variant thereof) (e.g., as described herein); a second linker; a polynucleotide encoding a reverse transcriptase (or functional fragment or variant thereof) (e.g., as described herein); a third linker; and a polynucleotide encoding an integrase (or a functional fragment or variant thereof) (e.g., as described herein).

### 8.3.8 Exemplary Editing Polypeptides

As described above, the editing polypeptides described herein are fusion proteins comprising (i) a DNA binding nickase (or functional fragment or variant thereof) (e.g., as described herein), (ii) a reverse transcriptase (or functional fragment or variant thereof) (e.g., as described herein), and (iii) an aptamer binding protein (or a functional fragment or variant thereof) (e.g., as described herein). In some embodiments, the editing polypeptide further comprises (iv) an integrase (or a functional fragment or variant thereof) (e.g., as described herein).

Exemplary editing polypeptides are described below. These are exemplary and are in no way limiting. The amino acid sequence of exemplary editing polypeptides is provided in Table 8.

In some embodiments, the editing polypeptide comprises an editing polypeptide set forth in Table 8. In some embodiments, the editing polypeptide comprises an amino acid sequence at least 90%, 91%, 92%, 93%, 94%, 95%, 96%, 97%, 98%, 99%, or 100% identical to the amino acid sequence of an editing polypeptide set forth in Table 8. In some embodiments, the editing polypeptide comprises an amino acid sequence at least 90%, 91%, 92%, 93%, 94%, 95%, 96%, 97%, 98%, 99%, or 100% identical to the amino acid sequence of SEQ ID NO: 138.

TABLE 8
The amino acid sequence of exemplary editing polypeptides.

| Description | Amino Acid Sequence | SEQ ID NO |
|---|---|---|
| MCP-Cas9-RT | MASNFTQFVLVDNGGTGDVTVAPSNFANGVAEWISS | 138 |
| | NSRSQAYKVTCSVRQSSAQKRKYTIKVEVPKVATQT | |
| | VGGVELPVAAWRSYLNMELTIPIFATNSDCELIVKA | |
| | MQGLLKDGNPIPSAIAANSGIYSAGGGGSGGGGSGG | |
| | GGSGMKRTADGSEFESPKKKRKVDKKYSIGLDIGTN | |
| | SVGWAVITDEYKVPSKKFKVLGNTDRHSIKKNLIGA | |
| | LLFDSGETAEATRLKRTARRRYTRRKNRICYLQEIF | |
| | SNEMAKVDDSFFHRLEESFLVEEDKKHERHPIFGNI | |
| | VDEVAYHEKYPTIYHLRKKLVDSTDKADLRLIYLAL | |
| | AHMIKFRGHFLIEGDLNPDNSDVDKLFIQLVQTYNQ | |
| | LFEENPINASGVDAKAILSARLSKSRRLENLIAQLP | |
| | GEKKNGLFGNLIALSLGLTPNFKSNEDLAEDAKLQL | |
| | SKDTYDDDLDNLLAQIGDQYADLFLAAKNLSDAILL | |
| | SDILRVNTEITKAPLSASMIKRYDEHHQDLTLLKAL | |
| | VRQQLPEKYKEIFFDQSKNGYAGYIDGGASQEEFYK | |
| | FIKPILEKMDGTEELLVKLNREDLLRKQRTEDNGSI | |
| | PHQIHLGELHAILRRQEDFYPFLKDNREKIEKILTF | |
| | RIPYYVGPLARGNSRFAWMTRKSEETITPWNFEEVV | |
| | DKGASAQSFIERMTNFDKNLPNEKVLPKHSLLYEYE | |
| | TVYNELTKVKYVTEGMRKPAFLSGEQKKAIVDLLEK | |
| | TNRKVTVKQLKEDYFKKIECFDSVEISGVEDRENAS | |
| | LGTYHDLLKIIKDKDELDNEENEDILEDIVLTLTLF | |
| | EDREMIEERLKTYAHLEDDKVMKQLKRRRYTGWGRL | |
| | SRKLINGIRDKQSGKTILDFLKSDGFANRNFMQLIH | |
| | DDSLTFKEDIQKAQVSGQGDSLHEHIANLAGSPAIK | |
| | KGILQTVKVVDELVKVMGRHKPENIVIEMARENQTT | |
| | QKGQKNSRERMKRIEEGIKELGSQILKEHPVENTQL | |
| | QNEKLYLYYLQNGRDMYVDQELDINRLSDYDVDAIV | |
| | PQSFLKDDSIDNKVLTRSDKNRGKSDNVPSEEVVKK | |
| | MKNYWRQLLNAKLITQRKEDNLTKAERGGLSELDKA | |
| | GFIKRQLVETRQITKHVAQILDSRMNTKYDENDKLI | |
| | REVKVITLKSKLVSDERKDFQFYKVREINNYHHAHD | |
| | AYLNAVVGTALIKKYPKLESEFVYGDYKVYDVRKMI | |
| | AKSEQEIGKATAKYFFYSNIMNFFKTEITLANGEIR | |
| | KRPLIETNGETGEIVWDKGRDFATVRKVLSMPQVNI | |
| | VKKTEVQTGGFSKESILPKRNSDKLIARKKDWDPKK | |
| | YGGFDSPTVAYSVLVVAKVEKGKSKKLKSVKELLGI | |
| | TIMERSSFEKNPIDFLEAKGYKEVKKDLIIKLPKYS | |
| | LFELENGRKRMLASAGELQKGNELALPSKYVNFLYL | |
| | ASHYEKLKGSPEDNEQKQLFVEQHKHYLDEIIEQIS | |
| | EFSKRVILADANLDKVLSAYNKHRDKPIREQAENII | |
| | HLFTLTNLGAPAAFKYFDTTIDRKRYTSTKEVLDAT | |
| | LIHQSITGLYETRIDLSQLGGDSGGSSGGSSGSETP | |
| | GTSESATPESSGGSSGGSSTLNIEDEYRLHETSKEP | |
| | DVSLGSTWLSDEPQAWAETGGMGLAVRQAPLIIPLK | |
| | ATSTPVSIKQYPMSQEARLGIKPHIQRLLDQGILVP | |
| | CQSPWNTPLLPVKKPGTNDYRPVQDLREVNKRVEDI | |
| | HPTVPNPYNLLSGLPPSHQWYTVLDLKDAFFCLRLH | |
| | PTSQPLFAFEWRDPEMGISGQLTWTRLPQGFKNSPT | |
| | LFNEALHRDLADFRIQHPDLILLQYVDDLLLAATSE | |
| | LDCQQGTRALLQTLGNLGYRASAKKAQICQKQVKYL | |
| | GYLLKEGQRWLTEARKETVMGQPTPKTPRQLREFLG | |
| | KAGFCRLFIPGFAEMAAPLYPLTKPGTLENWGPDQQ | |
| | KAYQEIKQALLTAPALGLPDLTKPFELFVDEKQGYA | |
| | KGVLTQKLGPWRRPVAYLSKKLDPVAAGWPPCLRMV | |
| | AAIAVLTKDAGKLTMGQPLVILAPHAVEALVKQPPD | |
| | RWLSNARMTHYQALLLDTDRVQFGPVVALNPATLLP | |
| | LPEEGLQHNCLDILAEAHGTRPDLTDQPLPDADHTW | |
| | YTDGSSLLQEGQRKAGAAVTTETEVIWAKALPAGTS | |
| | AQRAELIALTQALKMAEGKKLNVYTDSRYAFATAHI | |
| | HGEIYRRRGWLTSEGKEIKNKDEILALLKALFLPKR | |
| | LSIIHCPGHQKGHSAEARGNRMADQAARKAAITETP | |
| | DTSTLLIENSSPSGGSKRTADGSEFEPKKKRKV | |

### 8.3.9 Delivery of Editing Polypeptides

The editing polypeptides (and polynucleotides encoding them) described herein can be delivered to a cell or a population of cells by any suitable method known in the art. For example, via a polynucleotide encoding the editing polypeptide; via a vector (e.g., a plasmid or viral vector) comprising a polynucleotide encoding the editing polypeptide; via a particle (e.g., a viral particle, lipid particle, nanoparticle (e.g., a lipid nanoparticle)) encapsulating an editing polypeptide, a polynucleotide encoding an editing polypeptide, or a vector comprising a polynucleotide encoding the editing polypeptide. Methods of delivering each of the aforementioned are known to the person of ordinary skill in the art.

Also provided herein are pharmaceutical compositions comprising an editing polypeptide, a polynucleotide encoding an editing polypeptide; a vector (e.g., a plasmid or viral vector) comprising a polynucleotide encoding an editing polypeptide; a particle (e.g., a viral particle, lipid particle, nanoparticle (e.g., a lipid nanoparticle)) encapsulating an editing polypeptide, a polynucleotide encoding an editing polypeptide, or a vector comprising a polynucleotide encoding an editing polypeptide; and a pharmaceutically acceptable excipient. Suitable viral vectors are known in the art. Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors.

### 8.4 Trans-Template RNA (ttRNA)

In one aspect, provided herein are trans-template RNAs and methods of use in site-specific polynucleotide (e.g., gene, genome) engineering. The ttRNAs described herein comprise (i) a primer binding site (PBS), (ii) a reverse transcriptase template sequence, and (iii) at least one aptamer that is specifically recognized by a cognate aptamer binding protein (e.g., described herein). In some embodiments, the reverse transcriptase template sequence comprises an integration sequence encoding an integration site (e.g., for use in PASTE and PASTE-REPLACE methods of polynucleotide editing). In some embodiments, the reverse transcriptase template sequence comprises a target nucleotide modification (e.g., for use in PRIME methods of polynucleotide editing).

In some embodiments, the ttRNA does not contain a spacer, a scaffold, or a spacer and a scaffold. In some embodiments, the ttRNA cannot mediate targeting of a nuclease (e.g., a nickase) to a target polynucleotide.

As described above, the ttRNAs described herein comprise a PBS. The PBS binds to a complementary DNA sequence of the DNA flap with the 3′ OH group generated by the nickase, thereby providing a primer for the reverse transcriptase of the editing polypeptide (primers required for all known reverse transcriptases to initiate reverse transcription). In some embodiments, the PBS comprises or consists of from about 10-20, 10-19, 10-18, 10-17, 10-16, 10-15, 10-14, 10-13, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, or 14-15 nucleotides. In some embodiments, the PBS comprises or consists of about 20, 19, 18, 17, 16, 15, 14, 13, 12, 11, or 10 nucleotides. In some embodiments, the PBS is at the terminal 3′ end of the ttRNA.

In some embodiments, the ttRNA comprises additional elements, e.g., promoter(s). In some embodiments, the ttRNA comprises additional elements to mediate expression of the ttRNA (e.g., one or more promoter). In some embodiments, the ttRNA comprises a promoter. Promoters are well known to the person of ordinary skill in the art. Any suitable promoter may be utilized. In some embodiments, the ttRNA comprises a U6 promoter.

### 8.4.1 Reverse Transcriptase Template Sequence

As described above, the ttRNAs described herein comprise a reverse transcriptase template sequence. The reverse transcriptase template sequence serves as a template (i.e., encodes) the polynucleotide of interest (e.g., polynucleotide comprising, e.g., therapeutic nucleotide modification, diagnostic nucleotide modification; or e.g., a polynucleotide comprising an integration sequence encoding an integration site) for incorporation into a target polynucleotide (e.g., a gene or genome of a cell). In some embodiments, the reverse transcriptase template sequence comprises a therapeutic or diagnostic target nucleotide modification (e.g., in some embodiments a single nucleotide substitution, e.g., for use in PRIME editing methods). In some embodiments, the reverse transcriptase template sequence comprises an integration sequence comprising an integration site (e.g., as described herein, See, e.g., § 5.4.1.2) (e.g., for use in PASTE and PASTE-REPLACE methods).

### 8.4.1.1 Therapeutic and Diagnostic Nucleotide Modifications

In some embodiments, the reverse transcriptase template sequence comprises a polynucleotide of interest that comprises a therapeutic or diagnostic nucleotide modification (relative to the endogenous polynucleotide sequence, e.g., endogenous gene sequence). In some embodiments, the therapeutic or diagnostic nucleotide modification comprises at least one nucleotide insertion, deletion, or substitution. In some embodiments, the therapeutic or diagnostic nucleotide modification comprises an insertion, deletion, or substitution of from about 1-500, 1-200, 1-100, 1-50, 1-25, 1-10, 1-9, 1-8, 1-7, 1-6, 1-5, 1-4, 1-3, or 1-2 nucleotides. In some embodiments, the therapeutic or diagnostic nucleotide modification comprises a single nucleotide substitution.

A person of skill in the art will appreciate that while a therapeutic or diagnostic nucleotide modification will be encoded in the reverse transcriptase template sequence for PRIME editing applications; the therapeutic or diagnostic nucleotide modification will be encoded in a separate polynucleotide in PASTE and PASTE-REPLACE applications (see, e.g., § 5.6), as the reverse transcriptase template sequence will comprise an integration sequence encoding an integration site, as described below.

### 8.4.1.2 Integration Sequences and Integration Sites

In some embodiments, the compositions, systems, and methods described herein utilize an integration sequence (e.g., comprising an integration site) and a cognate integrase (e.g., as described herein, e.g., see § 5.3.4). Integration sequences, integration sites, and integrases are particularly useful in methods of PASTE editing (e.g., as described herein). In some embodiments, the ttRNA comprises an integration sequence encoding an integration site. Inclusion of the integration sequence encoding an integration site in the ttRNA allows for the incorporation of the integration site into a desired (site-specific) location in the polynucleotide (e.g., gene or genome) being edited.

It is understood by the person of ordinary skill in the art that integration sites and integrases for use in the compositions, systems, and methods described herein will be selected in pairs, wherein the selected integrase will specifically recognize the selected integration site. Exemplary integration sites include, but are not limited to, lox71 sites, attB sites, attP sites, attL sites, attR sites, Vox sites, FRT sites, or pseudo attP sites. The nucleotide sequence of exemplary integrations sites is provided in Table 9.

TABLE 9
Nucleotide sequence of exemplary integration sites.

| | | SEQ ID |
|---|---|---|
| Description | Nucleotide Sequence | NO |
| Lox71 | ATAACTTCGTATAATGTATGCTATACGAACGGTA | 139 |
| Lox66 | TACCGTTCGTATAATGTATGCTATACGAAGTTAT | 140 |
| attB | GGCCGGCTTGTCGACGACGGCGGTCTCCGTCGTCAGGAT | 141 |
| | CATCCGG | |
| attP | CCGGATGATCCTGACGACGGAGACCGCCGTCGTCGACAA | 142 |
| | GCCGGCC | |
| attB-TT | GGCTTGTCGACGACGGCGTTCTCCGTCGTCAGGATCAT | 143 |
| attP-TT | GTGGTTTGTCTGGTCAACCACCGCGTTCTCAGTGGTGTA | 144 |
| | CGGTACAAACCCA | |
| attB-AA | GGCTTGTCGACGACGGCGAACTCCGTCGTCAGGATCAT | 145 |
| attP-AA | GTGGTTTGTCTGGTCAACCACCGCGAACTCAGTGGTGTA | 146 |
| | CGGTACAAACCCA | |
| attB-CC | GGCTTGTCGACGACGGCGCCCTCCGTCGTCAGGATCAT | 147 |
| attP-CC | GTGGTTTGTCTGGTCAACCACCGCGCCCTCAGTGGTGTA | 148 |
| | CGGTACAAACCCA | |
| attB-GG | GGCTTGTCGACGACGGCGGGCTCCGTCGTCAGGATCAT | 149 |
| attP-GG | GTGGTTTGTCTGGTCAACCACCGCGGGCTCAGTGGTGTA | 150 |
| | CGGTACAAACCCA | |
| attB-TG | GGCTTGTCGACGACGGCGTGCTCCGTCGTCAGGATCAT | 151 |
| attP-TG | GTGGTTTGTCTGGTCAACCACCGCGTGCTCAGTGGTGTA | 152 |
| | CGGTACAAACCCA | |
| attB-GT | GGCTTGTCGACGACGGCGGTCTCCGTCGTCAGGATCAT | 153 |
| attP-GT | GTGGTTTGTCTGGTCAACCACCGCGGTCTCAGTGGTGTA | 154 |
| | CGGTACAAACCCA | |
| attB-CT | GGCTTGTCGACGACGGCGCTCTCCGTCGTCAGGATCAT | 155 |
| attP-CT | GTGGTTTGTCTGGTCAACCACCGCGCTCTCAGTGGTGTA | 156 |
| | CGGTACAAACCCA | |
| attB-CA | GGCTTGTCGACGACGGCGCACTCCGTCGTCAGGATCAT | 157 |
| attP-CA | GTGGTTTGTCTGGTCAACCACCGCGCACTCAGTGGTGTA | 158 |
| | CGGTACAAACCCA | |
| attB-TC | GGCTTGTCGACGACGGCGTCCTCCGTCGTCAGGATCAT | 159 |
| attP-TC | GTGGTTTGTCTGGTCAACCACCGCGTCCTCAGTGGTGTA | 160 |
| | CGGTACAAACCCA | |
| attB-GA | GGCTTGTCGACGACGGCGGACTCCGTCGTCAGGATCAT | 161 |
| attP-GA | GTGGTTTGTCTGGTCAACCACCGCGGACTCAGTGGTGTA | 162 |
| | CGGTACAAACCCA | |
| attB-AG | GGCTTGTCGACGACGGCGAGCTCCGTCGTCAGGATCAT | 163 |
| attP-AG | GTGGTTTGTCTGGTCAACCACCGCGAGCTCAGTGGTGTA | 164 |
| | CGGTACAAACCCA | |
| attB-AC | GGCTTGTCGACGACGGCGACCTCCGTCGTCAGGATCAT | 165 |
| attP-AC | GTGGTTTGTCTGGTCAACCACCGCGACCTCAGTGGTGTA | 166 |
| | CGGTACAAACCCA | |
| attB-AT | GGCTTGTCGACGACGGCGATCTCCGTCGTCAGGATCAT | 167 |
| attP-AT | GTGGTTTGTCTGGTCAACCACCGCGATCTCAGTGGTGTA | 168 |
| | CGGTACAAACCCA | |
| attB-GC | GGCTTGTCGACGACGGCGGCCTCCGTCGTCAGGATCAT | 169 |
| attP-GC | GTGGTTTGTCTGGTCAACCACCGCGGCCTCAGTGGTGTA | 170 |
| | CGGTACAAACCCA | |
| attB-CG | GGCTTGTCGACGACGGCGCGCTCCGTCGTCAGGATCAT | 171 |
| attP-CG | GTGGTTTGTCTGGTCAACCACCGCGCGCTCAGTGGTGTA | 172 |
| | CGGTACAAACCCA | |
| attB-TA | GGCTTGTCGACGACGGCGTACTCCGTCGTCAGGATCAT | 173 |
| attP-TA | GTGGTTTGTCTGGTCAACCACCGCGTACTCAGTGGTGTA | 174 |
| | CGGTACAAACCCA | |
| C31-attB | TGCGGGTGCCAGGGCGTGCCCTTGGGCTCCCCGGGCGCG | 175 |
| | TACTCC | |
| C31-attP | GTGCCCCAACTGGGGTAACCTTTGAGTTCTCTCAGTTGG | 176 |
| | GGG | |
| R4-attB | GCGCCCAAGTTGCCCATGACCATGCCGAAGCAGTGGTAG | 177 |
| | AAGGGCACCGGCAGACAC | |
| R4-attP | AGGCATGTTCCCCAAAGCGATACCACTTGAAGCAGTGGT | 178 |
| | ACTGCTTGTGGGTACACTCTGCGGGTGATGA | |
| BT1-attB | GTCCTTGACCAGGTTTTTGACGAAAGTGATCCAGATGAT | 179 |
| | CCAGCTCCACACCCCGAACGC | |
| BT1-attP | GGTGCTGGGTTGTTGTCTCTGGACAGTGATCCATGGGAA | 180 |
| | ACTACTCAGCACCACCAATGTTCC | |
| Bxb-attB | TCGGCCGGCTTGTCGACGACGGCGGTCTCCGTCGTCAGG | 181 |
| | ATCATCCGGGC | |
| Bxb-attP | GTCGTGGTTTGTCTGGTCAACCACCGCGGTCTCAGTGGT | 182 |
| | GTACGGTACAAACCCCGAC | |
| TG1-attB | GATCAGCTCCGCGGGCAAGACCTTCTCCTTCACGGGGTG | 183 |
| | GAAGGTC | |
| TG1-attP | TCAACCCCGTTCCAGCCCAACAGTGTTAGTCTTTGCTCT | 184 |
| | TACCCAGTTGGGCGGGATAGCCTGCCCG | |
| C1-attB | AACGATTTTCAAAGGATCACTGAATCAAAAGTATTGCTC | 185 |
| | ATCCACGCGAAATTTTTC | |
| C1-attP | AATATTTTAGGTATATGATTTTGTTTATTAGTGTAAATA | 186 |
| | ACACTATGTACCTAAAAT | |
| C370-attB | TGTAAAGGAGACTGATAATGGCATGTACAACTATACTCG | 187 |
| | TCGGTAAAAAGGCA | |
| C370-attP | TAAAAAAATACAGCGTTTTTCATGTACAACTATACTAGT | 188 |
| | TGTAGTGCCTAAA | |
| K38-attB | GAGCGCCGGATCAGGGAGTGGACGGCCTGGGAGCGCTAC | 189 |
| | ACGCTGTGGCTGCGGTC | |
| K38-attP | CCCTAATACGCAAGTCGATAACTCTCCTGGGAGCGTTGA | 190 |
| | CAACTTGCGCACCCTGA | |
| RB-attB | TCTCGTGGTGGTGGAAGGTGTTGGTGCGGGGTTGGCCGT | 191 |
| | GGTCGAGGTGGGGTGGTGGTAGCCATTCG | |
| RV-attP | GCACAGGTGTAGTGTATCTCACAGGTCCACGGTTGGCCG | 192 |
| | TGGACTGCTGAAGAACATTCCACGCCAGGA | |
| SPBC-attB | AGTGCAGCATGTCATTAATATCAGTACAGATAAAGCTGT | 193 |
| | ATCTCCTGTGAACACAATGGGTGCCA | |
| SPBC-attP | AAAGTAGTAAGTATCTTAAAAAACAGATAAAGCTGTATA | 194 |
| | TTAAGATACTTACTAC | |
| TP901-attB | TGATAATTGCCAACACAATTAACATCTCAATCAAGGTAA | 195 |
| | ATGCTTTTTCGTTTT | |
| TP901-attP | AATTGCGAGTTTTTATTTCGTTTATTTCAATTAAGGTAA | 196 |
| | CTAAAAAACTCCTTT | |
| Wβ-attB | AAGGTAGCGTCAACGATAGGTGTAACTGTCGTGTTTGTA | 197 |
| | ACGGTACTTCCAACAGCTGGCGTTTCAGT | |
| Wß-attP | TAGTTTTAAAGTTGGTTATTAGTTACTGTGATATTTATC | 198 |
| | ACGGTACCCAATAACCAATGAATATTTGA | |
| A118-attB | TGTAACTTTTTCGGATCAAGCTATGAAGGACGCAAAGAG | 199 |
| | GGAACTAAACACTTAATT | |
| A118-attP | TTGTTTAGTTCCTCGTTTTCTCTCGTTGGAAGAAGAAGA | 200 |
| | AACGAGAAACTAAAATTA | |
| BL3-attB | CAACCTGTTGACATGTTTCCACAGACAACTCACGTGGAG | 201 |
| | GTAGTCACGGCTTTTACGTTAGTT | |
| BL3-attP | GAGAATACTGTTGAACAATGAAAAACTAGGCATGTAGAA | 202 |
| | GTTGTTTGTGCACTAACTTTAA | |
| MR11-attB | ACAGGTCAACACATCGCAGTTATCGAACAATCTTCGAAA | 203 |
| | ATGTATGGAGGCACTTGTATCAATATAGGATGTATACCT | |
| | TCGAAGACACTTGTACATGATGGATTAGAAGGCAAATCC | |
| | TTT | |
| MR11-attP | CAAAATAAAAAACATTGATTTTTATTAACTTCTTTTGTG | 204 |
| | CGGAACTACGAACAGTTCATTAATACGAAGTGTACAAAC | |
| | TTCCATACAAAAATAACCACGACAATTAAGACGTGGTTT | |
| | CTA | |
| attL | ATTATTTCTCACCCTGA | 205 |
| attR | ATCATCTCCCACCCGGA | 206 |
| Vox | AATAGGTCTGAGAACGCCCATTCTCAGACGTATT | 207 |
| FRT | GAAGTTCCTATACTTTCTAGAGAATAGGAACTTC | 208 |
| Bxb1_attB_ | GGCCGGCTTGTCGACGACGGCGAACTCCGTCGTCAGGAT | 209 |
| 46_AA_site | CATCCGG | |
| Bxb1_attB_ | GGCCGGCTTGTCGACGACGGCGGACTCCGTCGTCAGGAT | 210 |
| 46_GA_site | CATCCGG | |
| Bxb1_attB_ | GGCCGGCTTGTCGACGACGGCGCACTCCGTCGTCAGGAT | 211 |
| 46_CA_site | CATCCGG | |
| Bxb1_attB_ | GGCCGGCTTGTCGACGACGGCGTACTCCGTCGTCAGGAT | 212 |
| 46_TA_site | CATCCGG | |
| Bxb1_attB_ | GGCCGGCTTGTCGACGACGGCGAGCTCCGTCGTCAGGAT | 213 |
| 46_AG_site | CATCCGG | |
| Bxb1_attB_ | GGCCGGCTTGTCGACGACGGCGGGCTCCGTCGTCAGGAT | 214 |
| 46_GG_site | CATCCGG | |
| Bxb1_attB_ | GGCCGGCTTGTCGACGACGGCGCGCTCCGTCGTCAGGAT | 215 |
| 46_CG_site | CATCCGG | |
| Bxb1_attB | GGCCGGCTTGTCGACGACGGCGTGCTCCGTCGTCAGGAT | 216 |
| 46_TG_site | CATCCGG | |
| Bxb1 attB | GGCCGGCTTGTCGACGACGGCGACCTCCGTCGTCAGGAT | 217 |
| 46 AC si | CATCCGG | |
| te | | |
| Bxb1_attB_ | GGCCGGCTTGTCGACGACGGCGGCCTCCGTCGTCAGGAT | 218 |
| 46_GC_site | CATCCGG | |
| Bxb1_attB_ | GGCCGGCTTGTCGACGACGGCGCCCTCCGTCGTCAGGAT | 219 |
| 46_CC_site | CATCCGG | |
| Bxb1_attB_ | GGCCGGCTTGTCGACGACGGCGTCCTCCGTCGTCAGGAT | 220 |
| 46_TC_site | CATCCGG | |
| Bxb1_attB_ | GGCCGGCTTGTCGACGACGGCGATCTCCGTCGTCAGGAT | 221 |
| 46_AT_site | CATCCGG | |
| Bxb1_attB_ | GGCCGGCTTGTCGACGACGGCGCTCTCCGTCGTCAGGAT | 222 |
| 46_CT_site | CATCCGG | |
| Bxb1_attB_ | GGCCGGCTTGTCGACGACGGCGTTCTCCGTCGTCAGGAT | 223 |
| 46_TT_site | CATCCGG | |
| Bxb1_attB_ | GGCTTGTCGACGACGGCGGTCTCCGTCGTCAGGATCAT | 224 |
| 38_GT_site | | |
| Bxb1_attB | GGCTTGTCGACGACGGCGAACTCCGTCGTCAGGATCAT | 225 |
| 38_AA_site | | |
| Bxb1_attB_ | GGCTTGTCGACGACGGCGGACTCCGTCGTCAGGATCAT | 226 |
| 38_GA_site | | |
| Bxb1_attB_ | GGCTTGTCGACGACGGCGCACTCCGTCGTCAGGATCAT | 227 |
| 38_CA_site | | |
| Bxb1_attB | GGCTTGTCGACGACGGCGTACTCCGTCGTCAGGATCAT | 228 |
| 38_TA_site | | |
| Bxb1_attB_ | GGCTTGTCGACGACGGCGAGCTCCGTCGTCAGGATCAT | 229 |
| 38_AG_site | | |
| Bxb1_attB_ | GGCTTGTCGACGACGGCGGGCTCCGTCGTCAGGATCAT | 230 |
| 38_GG_site | | |
| Bxb1_attB_ | GGCTTGTCGACGACGGCGCGCTCCGTCGTCAGGATCAT | 231 |
| 38_CG_site | | |
| Bxb1_attB_ | GGCTTGTCGACGACGGCGTGCTCCGTCGTCAGGATCAT | 232 |
| 38_TG_site | | |
| Bxb1_attB_ | GGCTTGTCGACGACGGCGACCTCCGTCGTCAGGATCAT | 233 |
| 38_AC_site | | |
| Bxb1_attB_ | GGCTTGTCGACGACGGCGGCCTCCGTCGTCAGGATCAT | 234 |
| 38_GC_site | | |
| Bxb1_attB_ | GGCTTGTCGACGACGGCGCCCTCCGTCGTCAGGATCAT | 235 |
| 38_CC_site | | |
| Bxb1_attB_ | GGCTTGTCGACGACGGCGTCCTCCGTCGTCAGGATCAT | 236 |
| 38_TC_site | | |
| Bxb1_attB_ | GGCTTGTCGACGACGGCGATCTCCGTCGTCAGGATCAT | 237 |
| 38_AT_site | | |
| Bxb1_attB_ | GGCTTGTCGACGACGGCGCTCTCCGTCGTCAGGATCAT | 238 |
| 38_CT_site | | |
| Bxb1_attB_ | GGCTTGTCGACGACGGCGTTCTCCGTCGTCAGGATCAT | 239 |
| 38_TT_site | | |
| Cre Lox 66 | TACCGTTCGTATAATGTATGCTATACGAAGTTAT | 240 |
| site | | |
| Cre Lox 71 | ATAACTTCGTATAATGTATGCTATACGAACGGTA | 241 |
| site | | |
| TP901-1 | TTTACCTTGATTGAGATGTTAATTGTG | 242 |
| minimal | | |
| attB site | | |
| TP901-1 | GCGAGTTTTTATTTCGTTTATTTCAATTAAGGTAACTAA | 243 |
| minimal | AAAACTCCTTT | |
| attP site | | |
| PhiBT1 | CTGGATCATCTGGATCACTTTCGTCAAAAACCTG | 244 |
| minimal | | |
| attB site | | |
| PhiBT1 | TTCGGGTGCTGGGTTGTTGTCTCTGGACAGTGATCCATG | 245 |
| minimal | GGAAACTACTCAGCACCA | |
| attP site | | |
| Pseudo attP | CCCCAACTGGGGTAACCTTTGAGTTCTCTCAGTTGGGG | 246 |
| site | | |

In some embodiments, the integration site comprises an integration site set forth in Table 9. In some embodiments, the integration site comprises a nucleotide sequence at least 95%, 96%, 97%, 98%, 99%, or 100% identical to the nucleotide sequence of an integration site set forth in Table 9. In some embodiments, the integration site comprises a nucleotide sequence comprising the nucleotide sequence of any one of the integration sites set forth in Table 9, and comprising 1, 2, or 3 nucleotide modifications. In some embodiments, the integration site comprises a nucleotide sequence comprising the nucleotide sequence of any one of the integration sites set forth in Table 9, and comprises no more than 1, 2, or 3 nucleotide modifications.

In some embodiments, the integration site comprises a nucleotide sequence at least 95%, 96%, 97%, 98%, 99%, or 100% identical to the nucleotide sequence of any one of SEQ ID NOS: 139-246. In some embodiments, the integration sequence comprises a nucleotide sequence comprising the nucleotide sequence of any one of SEQ ID NOS: 139-246, and comprising 1, 2, or 3 nucleotide modifications. In some embodiments, the integration sequence comprises a nucleotide sequence comprising the nucleotide sequence of any one of SEQ ID NOS: 139-246, and comprises no more than 1, 2, or 3 nucleotide modifications.

In some embodiments, integration site is integrated into a target polynucleotide with an efficiency of at least about 1%, 5%, 10%, 15%, 20%, 25%, 30%, 35%, 40%, 45%, or 50%. In some embodiments, integration is integrated into a target polynucleotide with an efficiency of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or any range that is formed from any two of those values as endpoints.

It is common knowledge to the person of ordinary skill in the art, that integration typically requires (e.g., as with serine integrases) an integration site (encoded by the ttRNA) and a recognition site (e.g., linked to a polynucleotide of interest for insertion) both of which are recognized by the integrase. The integration site can be inserted into the target polynucleotide (e.g., of a cell) using a nuclease (e.g., a nickase), a gRNA, and/or an integrase. A single or a plurality of integration sites can be added to a target polynucleotide (e.g., a genome). In some embodiments, one integration site is added to a target polynucleotide (e.g., a genome). In some embodiments, more than one integration site is added to a target polynucleotide (e.g., a genome). The recognition site may be operably linked to a target polynucleotide (e.g., gene of interest) in an exogenous DNA or RNA (e.g., as described herein).

To insert more than one unique polynucleotide (e.g., gene) of interest, each at a specific site, multiple orthogonal integrations sites can be added to the specific desired locations within the polynucleotide (e.g., genome) to mediate site-specific integration of the multiple polynucleotides. A first integration site is “orthogonal” to a second integration site when it does not significantly recognize the recognition site or the integrase (e.g., recombinase) recognized by the second integration site. Thus, for example, one attB site of an integrase (e.g., a recombinase) can be orthogonal to an attB site of a different recombinase (e.g., integrase). In addition, one pair of attB and attP sites of an integrase (e.g., a recombinase) can be orthogonal to another pair of attB and attP sites recognized by the same integrase (e.g., recombinase). A pair of recombinases are considered orthogonal to each other, as defined herein, when there is recognition of each other's attB or attP site sequences. In some embodiments, the same integrase (e.g., recombinase) or two different recombinases (e.g., integrases) recognize the same integration site less than 30%, 28%, 26%, 24%, 22%, 20%, 18%, 16%, 14%, 12%, 10%, 8%, 6%, 4%, 2%, or 1%, or any range that is formed from any two of those values as endpoints of the time.

A single or a plurality of integration sites can be added to a target polynucleotide (e.g., a genome). In some embodiments, one integration site is added to a target polynucleotide (e.g., a genome). In some embodiments, more than one integration site is added to a target polynucleotide (e.g., a genome).

The central dinucleotide of some integrases is involved in the association of the two paired integration sites. For example, the central dinucleotide of BxbINT is involved in the association of the AttB integration site with the AttP recognition site. Therefore, changing the matched central dinucleotide can modify the integrase activity and provide orthogonality for the insertion of multiple genes. Therefore, expanding the set of AttB/AttP dinucleotides can enable multiplex gene insertion using orthogonal sets of gRNAs and ttRNAs.

In some embodiments, the attB and/or attP site sequences comprise a central dinucleotide sequence. It has been shown that, for example, the central dinucleotide can be changed to GA from GT and that only GA containing attB/attP sites interact and will not cross react with GT containing sequences. In some embodiments, the central dinucleotide is selected from the group consisting of AG, AC, TG, TC, CA, CT, GA, AA, TT, CC, GG, AT, TA, GC, CG and GT. In some embodiments, the central dinucleotide is nonpalindromic. In some embodiments, the central dinucleotide is palindromic. In some embodiments, the integration site and the recognition site of a pair share the same central dinucleotide and can mediate recombination in the presence of the cognate integrase. Exemplary pairs of attB and attP integration and recognition sites are provided in Table 10.

TABLE 10
Pairs of exemplary attB and attP integration and recognition sites.
Each of the pairs comprises the same central dinucleotide (CD).

| Pair | attB | attP | CD |
|---|---|---|---|
| 1 | SEQ ID NO: 143 | SEQ ID NO: 144 | TT |
| 2 | SEQ ID NO: 145 | SEQ ID NO: 146 | AA |
| 3 | SEQ ID NO: 147 | SEQ ID NO: 148 | CC |
| 4 | SEQ ID NO: 149 | SEQ ID NO: 150 | GG |
| 5 | SEQ ID NO: 151 | SEQ ID NO: 152 | TG |
| 6 | SEQ ID NO: 153 | SEQ ID NO: 154 | GT |
| 7 | SEQ ID NO: 155 | SEQ ID NO: 156 | CT |
| 8 | SEQ ID NO: 157 | SEQ ID NO: 158 | CA |
| 9 | SEQ ID NO: 159 | SEQ ID NO: 160 | TC |
| 10 | SEQ ID NO: 161 | SEQ ID NO: 162 | GA |
| 11 | SEQ ID NO: 163 | SEQ ID NO: 164 | AG |
| 12 | SEQ ID NO: 165 | SEQ ID NO: 166 | AC |
| 13 | SEQ ID NO: 167 | SEQ ID NO: 168 | AT |
| 14 | SEQ ID NO: 169 | SEQ ID NO: 170 | GC |
| 15 | SEQ ID NO: 171 | SEQ ID NO: 172 | CG |
| 16 | SEQ ID NO: 173 | SEQ ID NO: 174 | TA |

### 8.4.2 Aptamers

As described above, the polynucleotide editing systems (e.g., gene editing systems) described herein utilize an aptamer and aptamer binding protein pair; wherein the ttRNA comprises an aptamer and the editing polypeptide comprises an aptamer binding protein (or a functional fragment or functional variant thereof) that specifically recognizes (i.e., binds to) the aptamer within the ttRNA. The use of the aptamer/aptamer binding protein pair allows for recruitment of the ttRNA directly to the location on the polynucleotide that is to be targeted by the editing system through the binding of the aptamer to the aptamer binding protein (that is itself targeted by fusion to the DNA binding nickase (e.g., described herein) which is in turn targeted to the target location on the polynucleotide through binding to a gRNA comprising the spacer and a scaffold (e.g., as described herein). Any suitable aptamer/aptamer binding protein pair known to the person of ordinary skill in the art may be employed. Exemplary pairs include, but are not limited to, an MS2 aptamer/MS2 coat protein (MCP) pair, a Qβ aptamer/Qβ coat protein pair, and a PP7 aptamer/PP7 coat protein. The aptamers are further detailed below; and the corresponding aptamer binding proteins are further detailed in § 5.3.3.

Exemplary aptamers include, but are not limited to, an MS2 RNA aptamer, a Qβ aptamer, and a PP7 aptamer. As discussed above the MS2 aptamer is specifically recognized (i.e., bound by) MCP; the Qβ aptamer is specifically recognized (i.e., bound by) the Qβ coat protein; and the PP7 aptamer is specifically recognized (i.e., bound by) the PP7 coat protein. The RNA sequence of exemplary aptamer suitable for use in the RNAs (e.g., ttRNAs) described herein is provided in Table 11.

TABLE 11
RNA sequence of exemplary aptamers.

| Description | RNA Sequence | SEQ ID NO |
|---|---|---|
| MS2 Aptamer | AACATGAGGATCACCCATGTC | 247 |
| Qβ RNA | ATGCATGTCTAAGACAGCAT | 248 |
| Aptamer | | |
| PP7 RNA | TAAGGAGTTTATATGGAAACCCTTA | 249 |
| Aptamer | | |

Functional variants or fragments of aptamers (compared to a reference sequence) may be utilized, as long as the fragments or variants are capable of forming an RNA hairpin loop structure that can be recognized (i.e., bound by) the cognate aptamer binding protein.

In some embodiments, the aptamer comprises or is an aptamer set forth in Table 11. In some embodiments, the aptamer comprises a nucleotide sequence at least 95%, 96%, 97%, 98%, 99%, or 100% identical to the nucleotide sequence of an aptamer set forth in Table 11. In some embodiments, the aptamer comprises a nucleotide sequence comprising the nucleotide sequence of any one of the aptamers set forth in Table 11, and comprising 1, 2, or 3 nucleotide modifications. In some embodiments, the aptamer comprises a nucleotide sequence comprising the nucleotide sequence of any one of the aptamers set forth in Table 11, and comprises no more than 1, 2, or 3 nucleotide modifications.

In some embodiments, the aptamer comprises a nucleotide sequence at least 95%, 96%, 97%, 98%, 99%, or 100% identical to the nucleotide sequence of any one of SEQ ID NOS: 247-249. In some embodiments, the aptamer comprises a nucleotide sequence comprising the nucleotide sequence of any one of SEQ ID NOS: 247-249, and comprising 1, 2, or 3 nucleotide modifications. In some embodiments, the aptamer comprises a nucleotide sequence comprising the nucleotide sequence of any one of SEQ ID NOS: 247-249, and comprises no more than 1, 2, or 3 nucleotide modifications.

In some embodiments, the ttRNA comprises 1, 2, 3, 4, 5, or more aptamers. In some embodiments, the ttRNA comprises at least 2, 3, 4, or 5 aptamers. In some embodiments, the ttRNA comprises an aptamer at the 3′ end of the ttRNA. In some embodiments, the ttRNA comprises an aptamer at the 5′ end of the ttRNA. In some embodiments, the ttRNA comprises an aptamer at the 3′ end and 5′ end the of the ttRNA.

### 8.4.3 Orientation

As described above, ttRNAs described herein comprise (i) a primer binding site, (ii) a reverse transcriptase template sequence, and (iii) at least one aptamer that is specifically recognized by a cognate aptamer binding protein (e.g., described herein). The components can be arranged in any order as long as each component maintains its function (e.g., as described herein.

In some embodiments, the ttRNA comprises from 5′ to 3′ a primer binding site, a reverse transcriptase template sequence, and at least one aptamer that is specifically recognized by a cognate aptamer binding protein (e.g., described herein). In some embodiments, the ttRNA comprises from 5′ to 3′ a promoter (e.g., a U6 promoter), a primer binding site, a reverse transcriptase template sequence, and at least one aptamer that is specifically recognized by a cognate aptamer binding protein (e.g., described herein).

In some embodiments, the ttRNA comprises from 5′ to 3′ at least one aptamer that is specifically recognized by a cognate aptamer binding protein (e.g., described herein), a primer binding site, and a reverse transcriptase template sequence. In some embodiments, the ttRNA comprises from 5′ to 3′ a promoter (e.g., a U6 promoter), at least one aptamer that is specifically recognized by a cognate aptamer binding protein (e.g., described herein), a primer binding site, and a reverse transcriptase template sequence.

In some embodiments, the ttRNA comprises from 5′ to 3′ at least one aptamer that is specifically recognized by a cognate aptamer binding protein (e.g., described herein), a primer binding site, a reverse transcriptase template sequence, and at least one aptamer that is specifically recognized by a cognate aptamer binding protein (e.g., described herein). In some embodiments, the ttRNA comprises from 5′ to 3′ a promoter (e.g., a U6 promoter), at least one aptamer that is specifically recognized by a cognate aptamer binding protein (e.g., described herein), a primer binding site, a reverse transcriptase template sequence, and at least one aptamer that is specifically recognized by a cognate aptamer binding protein (e.g., described herein).

### 8.4.4 Exemplary ttRNAs

As described above, ttRNAs described herein comprise (i) a primer binding site, (ii) a reverse transcriptase template sequence, and (iii) at least one aptamer that is specifically recognized by a cognate aptamer binding protein (e.g., described herein). Exemplary ttRNAs are described below. These are exemplary and are in no way limiting. The nucleic acid sequence of exemplary editing polypeptides is provided in Table 12.

TABLE 12
The nucleic acid sequence of exemplary ttRNAs.

| Description | Nucleic Acid Sequence | SEQ ID NO |
|---|---|---|
| Left MS2 | aacatgaggatcacccatgtcGACGAGCGCGGCGAT | 250 |
| ACTB | ATCATCATCCATGGccggATGATCCTGACGACGGag | |
| template | accgccgtcgtcgacaagccggccTGAGCTGCGAGA | |
| | A | |
| Left MS2 | aacatgaggatcacccatgtcCGGGGGTCGCAGTCG | 251 |
| LMNB1 | CCATGatgatcctgacgacggagaccgccgtcgtcg | |
| template | acaagccCGGGCGGCG | |
| Right MS2 | GACGAGCGCGGCGATATCATCATCCATGGccggATG | 252 |
| ACTB | ATCCTGACGACGGagaccgccgtcgtcgacaagccg | |
| template | gccTGAGCTGCGAGAAaacatgaggatcacccatgt | |
| | c | |
| Right MS2 | CGGGGGTCGCAGTCGCCATGatgatcctgacgacgg | 253 |
| LMNB1 | agaccgccgtcgtcgacaagccCGGGCGGCGaacat | |
| template | gaggatcacccatgtc | |
| Both MS2 | aacatgaggatcacccatgtcGACGAGCGCGGCGAT | 254 |
| ACTB | ATCATCATCCATGGccggATGATCCTGACGACGGag | |
| template | accgccgtcgtcgacaagccggccTGAGCTGCGAGA | |
| | Aaacatgaggatcacccatgtc | |
| Both MS2 | aacatgaggatcacccatgtcCGGGGGTCGCAGTCG | 255 |
| LMNB1 | CCATGatgatcctgacgacggagaccgccgtcgtcg | |
| template | acaagccCGGGCGGCGaacatgaggatcacccatgt | |
| | c | |

### 8.4.5 Delivery of Trans-Template RNA

The ttRNAs described herein can be delivered to a cell or a population of cells by any suitable method known in the art. For example, via an RNA polynucleotide; via a vector (e.g., a plasmid or viral vector) comprising an RNA polynucleotide; via a particle (e.g., a viral particle, lipid particle, nanoparticle (e.g., a lipid nanoparticle)) encapsulating the polynucleotide or vector. Methods of delivering each of the aforementioned are known to the person of ordinary skill in the art. Also provided herein are pharmaceutical compositions comprising a ttRNA polynucleotide; a vector (e.g., a plasmid or viral vector) comprising the polynucleotide; a particle (e.g., a viral particle, lipid particle, nanoparticle (e.g., a lipid nanoparticle)) encapsulating the polynucleotide; and a pharmaceutically acceptable excipient.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors.

### 8.5 gRNAs

In some embodiments, the compositions, systems, and methods described herein comprise or utilize a gRNA. A gRNA typically functions to guide the insertion or deletion of one or more polynucleotides of interest (e.g., a gene of interest) into a target polynucleotide (e.g., genome). In some embodiments, the gRNA molecule is naturally occurring. In some embodiments, a gRNA molecule is non-naturally occurring. In some embodiments, a gRNA molecule is a synthetic gRNA molecule. In some embodiments, the gRNA comprises one or nucleotide modifications (e.g., to improve stability and/or half-life after being introduced into a cell).

### 8.5.1 Targeting gRNAs

In some embodiments, the compositions, systems, and methods described herein comprise a targeting gRNA that comprises a spacer and a scaffold. The targeting gRNA functions to guide an editing polypeptide (e.g., an editing polypeptide described herein, see, e.g., § 5.3) to the target polynucleotide (e.g., a specific target sequence within a genome, e.g., within a cell). In some embodiments, the targeting gRNA comprises about 90-110, 95-105, 95-100, or 100-105 nucleotides. In some embodiments, the targeting gRNA comprises about 90, 91, 92, 93, 94, 95, 96, 97, 98, 99, 100, 101, 102, 103, 104, 105, 106, 107, 108, 109, or 100 nucleotides. In some embodiments, the spacer comprises from about 18-25, 18-24, 18-23, 18-22, 18-21, 18-20, 19-25, 19-24, 19-23, 19-22, 19-21, or 19-20 nucleotides. In some embodiments, the spacer comprises about 16, 17, 18, 19, 20, 21, 22, 23, or 24 nucleotides.

In some embodiments, the targeting gRNA does not contain one, two, or three of the following: a primer binding site, a reverse transcriptase template sequence, or an aptamer that is specifically recognized by a cognate aptamer binding protein.

### 8.5.2 Nicking gRNAs

In some embodiments, the compositions, systems, and methods described herein comprise or utilize one or more a nicking gRNA (ngRNA). In some embodiments, the ngRNA targets a nickase such that the nickase can induce a nick in a DNA polynucleotide at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120

Use of a ngRNA can increase the editing efficiency, wherein the cell remakes the non-edited strand using the edited strand as the template to induce homologous recombination. In some embodiments, the non-edited strand is nicked using a ngRNA (e.g., described herein). In some embodiments, nicking of the non-edited strand can increase editing efficiency by about 1-, 2-, 3-, 4-, or 5-fold or more (relative to the editing efficiency in the absence of the ngRNA). Although the optimal nicking position varies depending on the genomic site, nicks positioned 3′ of the edit about 40-90 base pairs from the nick induced by the nickase can generally increase editing efficiency without excess indel formation. A person of ordinary skill in the art, may for example, start testing non-edited strand nicks about 50 base pairs from the nickase induced nick, and testing alternative nick locations (indel frequencies exceed acceptable levels).

### 8.5.3 Paired gRNAs

In some embodiments, the compositions, systems, and methods described herein comprise or utilize one or more set of paired guides that allow for the simultaneous deletion of an endogenous polynucleotide (e.g., gene) and insertion of a polynucleotide of interest (e.g., modified gene). The target dsDNA comprises two protospacers each on opposite strands of the target dsDNA. One gRNA (e.g., targeting gRNA) is targeted to one strand, while the other gRNA (e.g., targeting gRNA) of the pairs is targeted to the opposite strand. The targeting gRNA:editing polypeptide complex generates a single strand nick at each target site.

### 8.5.4 Modification of gRNAs

In some embodiments, the gRNA comprises one or nucleotide modifications (e.g., to improve stability and/or half-life after being introduced into a cell). In some embodiments, chemical modifications on the ribose rings and phosphate backbone of gRNAs are incorporated. Ribose modifications are typically placed at the 2′OH as it is readily available for manipulation. Simple modifications at the 2′OH include 2′-O-methyl, 2′-fluoro, and 2′-deoxy-2′-fluoro-beta-D-arabinonucleic acid (2′fluoro-ANA). More extensive ribose modifications such as 2′F-4′-Cα-OMe and 2′,4′-di-Cα-OMe combine modification at both the 2′ and 4′ carbons. Exemplary phosphodiester modifications include sulfide-based phosphorothioate (PS) or acetate-based phosphonoacetate alterations. Combinations of the ribose and phosphodiester modifications can also be utilized such as 2′-O-methyl 3′phosphorothioate (MS), or 2′-O-methyl-3′-thioPACE (MSP), and 2′-O-methyl-3′-phosphonoacetate (MP) RNAs. Locked and unlocked nucleotides such as locked nucleic acid (LNA), bridged nucleic acids (BNA), S-constrained ethyl (cEt), and unlocked nucleic acid (UNA) are examples of sterically hindered nucleotide modifications that can also be utilized.

### 8.5.5 Delivery of gRNAs

The gRNAs described herein (e.g., targeting gRNAs, ngRNAs) can be delivered to a cell or a population of cells by any suitable method known in the art. For example, via an RNA polynucleotide; via a vector (e.g., a plasmid or viral vector) comprising an RNA polynucleotide; via a particle (e.g., a viral particle, lipid particle, nanoparticle (e.g., a lipid nanoparticle)) encapsulating the polynucleotide or vector. Methods of delivering each of the aforementioned are known to the person of ordinary skill in the art. Also provided herein are pharmaceutical compositions comprising a gRNA described herein (e.g., targeting gRNA, ngRNA) polynucleotide; a vector (e.g., a plasmid or viral vector) comprising the polynucleotide; a particle (e.g., a viral particle, lipid particle, nanoparticle (e.g., a lipid nanoparticle)) encapsulating the polynucleotide; and a pharmaceutically acceptable excipient.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors.

### 8.6 PASTE and PASTE-REPLACE Polynucleotide of Interest

A person of skill in the art will appreciate that while a polynucleotide of interest (e.g., for therapeutic or diagnostic purposes) will be encoded in the reverse transcriptase template sequence for PRIME editing applications (see, e.g., § 5.4.1.1); the polynucleotide of interest (e.g., for therapeutic or diagnostic purposes) will be encoded in a separate polynucleotide in PASTE and PASTE-REPLACE applications, as the reverse transcriptase template sequence will comprise an integration sequence encoding an integration site.

In some embodiments, the polynucleotide of interest (e.g., for therapeutic or diagnostic purposes) comprises at least one nucleotide insertion, deletion, or substitution compared to the endogenous sequence of the target polynucleotide. In some embodiments, the polynucleotide of interest (e.g., for therapeutic or diagnostic purposes) comprises an insertion, deletion, or substitution of from about 1-500, 1-200, 1-100, 1-50, 1-25, 1-10, 1-9, 1-8, 1-7, 1-6, 1-5, 1-4, 1-3, or 1-2 nucleotides.

The size of the inserted polynucleotide of interest (e.g., gene) can vary from about 1 to about 50,000 nucleotides. In some embodiments, the inserted polynucleotide (e.g., gene) comprises or consists of from about 1-50000, 1-25000, 1-20000, 1-15000, 1-10000, 1-5000, 1-25000, 1-2000, 1-1000, 1-100, 1-50, 1-25, 1-10, 1-5, or 1-2 nucleotides.

In some embodiments, the inserted polynucleotide of interest (e.g., gene) comprises or consists of about 1, 10, 50, 100, 150, 200, 250, 300, 350, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, 9000, 9200, 9400, 9600, 9800, 10,000, 10,400, 10,600, 10,800, 11,000, 11,200, 11,400, 11,600, 11,800, 12,000, 14,000, 16,000, 18,000, 20,000, 30,000, 40,000, 50,000 nucleotides, or any range that is formed from any two of those values as endpoints.

In some embodiments, a plurality (e.g., 2 or more) polynucleotides of interest encoding different target nucleotide modifications (e.g., encoding different genes) are utilized. This process is referred to herein as multiplexing. The site-specific integration of different polynucleotides of interest can be accomplished utilizing distinct pairs of integration sites and integrases as described in § 5.4.1.2. In some embodiments, at least 1, 2, 3, 4, 5, 6, 7, 8, 9, or 10 different polynucleotides of interest are inserted into the target polynucleotide (e.g., genome of a cell). In some embodiments, multiplexing allows integration of for example, signaling cascade, over-expression of a protein of interest with its cofactor, insertion of multiple genes mutated in a neoplastic condition, or insertion of multiple chimeric antigen receptors for treatment of cancer.

In some embodiments, the polynucleotide(s) of interest is delivered into a cell via a polynucleotide (e.g., DNA, RNA), a minicircle (e.g., DNA or RNA), a vector comprising the polynucleotide (e.g., a plasmid or viral vector), a polypeptide, or a particle encapsulating the polynucleotide, minicircle, vector, or polypeptide.

In some embodiments, the polynucleotide of interest encodes, e.g., a lysosomal enzyme), a blood factor (e.g., Factor I, II, V, VII, X, XI, XII or XIII), a membrane protein, an exon, an intracellular protein (e.g., a cytoplasmic protein, a nuclear protein, an organellar protein such as a mitochondrial protein or lysosomal protein), an extracellular protein, a structural protein, a signaling protein, a regulatory protein, a transport protein, a sensory protein, a motor protein, a defense protein, or a storage protein, an anti-inflammatory protein, or a pro-inflammatory protein. Such polynucleotides of interest may be useful in methods of treating diseases certain diseases.

In some embodiments, the polynucleotide of interest is not for therapeutic use but for diagnostic use, e.g., such as a reporter gene upstream or downstream of a gene targeted for genetic analyses, such as, without limitation, for determining the expression of a gene. In some embodiments, the polynucleotide of interest can be used in plant genetics to insert genes to enhance drought tolerance, weather hardiness, and increased yield and herbicide resistance in plants.

### 8.7 Compositions, Pharmaceutical Compositions, Systems, and Kits

Provided herein are compositions (including pharmaceutical compositions), systems, and kits comprising any one or more (e.g., all) of the components described herein (e.g., an editing polypeptide, a ttRNA, one of more gRNAs (e.g., targeting gRNA, ngRNA), polynucleotide inserts). In one aspect, provided herein is a system comprising at least two components of an editing system described herein (e.g., an editing polypeptide, a gRNA, a ttRNA). In one aspect, provided herein are compositions comprising at least one components of an editing system described herein (e.g., an editing polypeptide, a gRNA, a ttRNA). In one aspect, provided herein are compositions comprising at least one components of an editing system described herein (e.g., an editing polypeptide, a gRNA, a ttRNA).

### 8.7.1 Pharmaceutical Compositions

Pharmaceutical compositions descried herein comprise at least one component of an editing system described herein (e.g., an editing polypeptide) and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing at least one component of an editing system described herein (e.g., an editing polypeptide) and formulating it into a pharmaceutically acceptable composition by the addition of one or more pharmaceutically acceptable excipient. In some embodiments, the pharmaceutical composition comprises a single component described herein (e.g., an editing polypeptide). In some embodiments, the pharmaceutical composition comprises a plurality of the components described herein (e.g., an editing polypeptide, a ttRNA, a targeting gRNA, etc.).

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. The skilled person knows the various possibilities to administer a pharmaceutical composition described herein a in order to deliver the editing system or composition to a target cell. Non-limiting embodiments include parenteral administration, such as intramuscular, intradermal, subcutaneous, transcutaneous, or mucosal administration. In one embodiment, the pharmaceutical composition is formulated for intravenous administration. In one embodiment, the pharmaceutical composition is formulated for administration by intramuscular, intradermal, or subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients (e.g., carriers) used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; orsodium hydroxide, hydrochloric acid, citric acid, or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 8.7.2 Kits

Also provided herein are kits comprising at least one pharmaceutical composition described herein. In addition, the kit may comprise a liquid vehicle for solubilizing or diluting, and/or technical instructions. The technical instructions of the kit may contain information about administration and dosage and subject groups. In some embodiments, the kit contains a single container comprising a single pharmaceutical composition described herein. In some embodiments, the kit at least two separate containers, each comprising a different pharmaceutical composition described herein (e.g., a first container comprising a pharmaceutical composition comprising one component of an editing system described herein, e.g., an editing polypeptide described herein, and a second container comprising a second pharmaceutical composition comprising a second component of an editing system described herein, e.g., a ttRNA).

### 8.8 Methods of Use

Provided herein are various methods of using the editing systems, compositions, pharmaceutical compositions described herein and any one or more of the components thereof (e.g., an editing polypeptide).

In one aspect, provided herein are methods of editing a target polynucleotide, the method comprising contacting the target polynucleotide with an editing system, composition, pharmaceutical composition, or any component thereof (e.g., an editing polypeptide). In some embodiments, the target polynucleotide is or is within a gene. In some embodiments, the target polynucleotide is or is within a genome.

In one aspect, provided herein are methods of editing a target polynucleotide within a cell, the method comprising introducing into the cell an editing system, composition, pharmaceutical composition, or any component thereof (e.g., an editing polypeptide). In some embodiments, the target polynucleotide is or is within a gene. In some embodiments, the target polynucleotide is or is within a genome.

In one aspect, provided herein are methods of editing a target polynucleotide within a cell in a subject, the method comprising administering to the subject an editing system, composition, pharmaceutical composition, or any component thereof (e.g., an editing polypeptide), in an amount sufficient to deliver the editing system, composition, pharmaceutical composition, or component to a cell in the subject. In some embodiments, the target polynucleotide is or is within a gene. In some embodiments, the target polynucleotide is or is within a genome.

In one aspect, provided herein are methods of delivering an editing system, composition, pharmaceutical composition, or any component thereof (e.g., an editing polypeptide) to a cell comprising contacting the cell with the editing system, composition, pharmaceutical composition, or component thereof, in an amount sufficient to deliver the editing system, composition, pharmaceutical composition, or any component thereof to the cell.

In one aspect, provided herein are methods of delivering an editing system, composition, pharmaceutical composition, or any component thereof (e.g., an editing polypeptide) to a subject, the method comprising administering the editing system, composition, pharmaceutical composition, or component thereof to the subject.

In one aspect, provided herein are methods of delivering an editing system, composition, pharmaceutical composition, or any component thereof (e.g., an editing polypeptide) to a cell in a subject, the method comprising administering the editing system, composition, pharmaceutical composition, or component thereof to the subject, in an amount sufficient to deliver the editing system, composition, pharmaceutical composition, or component to a cell in the subject.

In one aspect, provided herein are methods of treating a subject diagnosed with or suspected of having a disease associated with a genetic mutation comprising administering a composition or system described herein to the subject in an amount sufficient to correct the genetic mutation. Exemplary diseases associated with a genetic mutation, include, but are not limited to cystic fibrosis, muscular dystrophy, hemochromatosis, Tay-Sachs, Huntington disease, Congenital Deafness, Sickle cell anemia, Familial hypercholesterolemia, adenosine deaminase (ADA) deficiency, X-linked SCID (X-SCID), and Wiskott-Aldrich syndrome (WAS).

In some embodiments, the genetic mutation is in one of the following genes: GBA, BTK, ADA, CNGB3, CNGA3, ATF6, GNAT2, ABCA1, ABCA7, APOE, CETP, LIPC, MMP9, PLTP, VTN, ABCA4, MFSD8, TLR3, TLR4, ERCC6, HMCN1, HTRA1, MCDR4, MCDR5, ARMS2, C2, C3, CFB, CFH, JAG1, NOTCH2, CACNA1F, SERPINA1, TTR, GSN, B2M, APOA2, APOA1, OSMR, ELP4, PAX6, ARG, ASL, PITX2, FOXC1, BBS1, BBS10, BBS2, BBS9, MKKS, MKS1, BBS4, BBS7, TTC8, ARL6, BBS5, BBS12, TRIM32, CEP290, ADIPOR1, BBIP1, CEP19, IFT27, LZTFL1, DMD, BEST1, HBB, CYP4V2, AMACR, CYP7B1, HSD3B7, AKR1D1, OPN1SW, NR2F1, RLBP1, RGS9, RGS9BP, PROM1, PRPH2, GUCY2D, CACD, CHM, ALAD, ASS1, SLC25A13, OTC, ACADVL, ETFDH, TMEM67, CC2D2A, RPGRIP1L, KCNV2, CRX, GUCA1A, CERKL, CDHR1, PDE6C, TTLL5, RPGR, CEP78, C21 orf2, C8ORF37, RPGRIP1, ADAM9, POC1B, PITPNM3, RAB28, CACNA2D4, AIPL1, UNC119, PDE6H, OPN1LW, RIMS1, CNNM4, IFT81, RAX2, RDH5, SEMA4A, CORD17, PDE6B, GRK1, SAG, RHO, CABP4, GNB3, SLC24A1, GNAT1, GRM6, TRPM1, LRIT3, TGFBI, TACSTD2, KRT12, OVOL2, CPS1, UGT1A1, UGT1A9, UGT1A8, UGT1A7, UGT1A6, UGT1A5, UGT1A4, CFTR, DLD, EFEMP1, ABCC2, ZNF408, LRP5, FZD4, TSPAN12, EVR3, APOB, SLC2A2, LOC106627981, GBA1, NR2E3, OAT, SLC40A1, F8, F9, UROD, CPDX, HFE, JH, LDLR, EPHX1, TJP2, BAAT, NBAS, LARS1, HAMP, HJV, RS1, ADAMTS18, LRAT, RPE65, LCA5, MERTK, GDF6, RD3, CCT2, CLUAP1, DTHD1, NMNAT1, SPATA7, IFT140, IMPDH1, OTX2, RDH12, TULP1, CRB1, MT-ND4, MT-ND1, MT-ND6, BCKDHA, BCKDHB, DBT, MMAB, ARSB, GUSB, NAGS, NPC1, NPC2, NDP, OPA1, OPA3, OPA4, OPA5, RTN4IP1, TMEM126A, OPA6, OPA8, ACO2, PAH, PRKCSH, SEC63, GAA, UROS, PPDX, HPX, HMOX1, HMBS, MIR223, CYP1B1, LTBP2, AGXT, ATP8B1, ABCB11, ABCB4, FECH, ALAS2, PRPF31, RP1, EYS, TOPORS, USH2A, CNGA1, C2ORF71, RP2, KLHL7, ORF1, RP6, RP24, RP34, ROM1, ADGRA3, AGBL5, AHR, ARHGEF18, CA4, CLCC1, DHDDS, EMC1, FAM161A, HGSNAT, HK1, IDH3B, KIAA1549, KIZ, MAK, NEUROD1, NRL, PDE6A, PDE6G, PRCD, PRPF3, PRPF4, PRPF6, PRPF8, RBP3, REEP6, SAMD11, SLC7A14, SNRNP200, SPP2, ZNF513, NEK2, NEK4, NXNL1, OFD1, RP1L1, RP22, RP29, RP32, RP63, RP9, RGR, POMGNT1, DHX38, ARL3, COL2A1, SLCO1B1, SLCO1B3, KCNJ13, TIMP3, ELOVL4, TFR2, FAH, HPD, MYO7A, CDH23, PCDH15, DFNB31, GPR98, USH1C, USH1G, CIB2, CLRN1, HARS, ABHD12, ADGRV1, ARSG, CEP250, IMPG1, IMPG2, VCAN, G6PC1, ATP7B.

### EXAMPLES

### 9.1 Example 1. Integration Recognition Site Insertion with Trans-Template RNA (ttRNA)

Eight different ttRNAs were generated. Each of the ttRNAs generated contained either no MS2 aptamers, an MS2 aptamer at the 3′ end (left), the 5′ end (right), or both the 3′ end and 5′ end (both sides) of the ttRNA. The amino acid sequence of each of the 8 ttRNAs generated is provided in Table 13. Expression of each of the ttRNAs was driven by a U6 promoter and contained a primer binding site, a reverse transcriptase template sequence, and a complement of an integration sequence.

TABLE 13
Nucleic acid sequence of ttRNAs.

| Description | Nucleic Acid Sequence | SEQ ID NO |
|---|---|---|
| No MS2 ACTB template | GACGAGCGCGGCGATATCATCATCCATGG | 256 |
| | ccggATGATCCTGACGACGGagaccgccg | |
| | tcgtcgacaagccggccTGAGCTGCGAGA | |
| | A | |
| No MS2 LMNB1 template | CGGGGGTCGCAGTCGCCATGatgatcctg | 257 |
| | acgacggagaccgccgtcgtcgacaagcc | |
| | CGGGCGGCG | |
| Left MS2 ACTB template | aacatgaggatcacccatgtcGACGAGCG | 258 |
| | CGGCGATATCATCATCCATGGccggATGA | |
| | TCCTGACGACGGagaccgccgtcgtcgac | |
| | aagccggccTGAGCTGCGAGAA | |
| Left MS2 LMNB1 | aacatgaggatcacccatgtcCGGGGGTC | 259 |
| template | GCAGTCGCCATGatgatcctgacgacgga | |
| | gaccgccgtcgtcgacaagccCGGGCGGC | |
| | G | |
| Right MS2 ACTB | GACGAGCGCGGCGATATCATCATCCATGG | 260 |
| template | ccggATGATCCTGACGACGGagaccgccg | |
| | tcgtcgacaagccggccTGAGCTGCGAGA | |
| | Aaacatgaggatcacccatgtc | |
| Right MS2 LMNB1 | CGGGGGTCGCAGTCGCCATGatgatcctg | 261 |
| template | acgacggagaccgccgtcgtcgacaagcc | |
| | CGGGCGGCGaacatgaggatcacccatgt | |
| | c | |
| Both MS2 ACTB template | aacatgaggatcacccatgtcGACGAGCG | 262 |
| | CGGCGATATCATCATCCATGGccggATGA | |
| | TCCTGACGACGGagaccgccgtcgtcgac | |
| | aagccggccTGAGCTGCGAGAAaacatga | |
| | ggatcacccatgtc | |
| Both MS2 LMNB1 | aacatgaggatcacccatgtcCGGGGGTC | 263 |
| template | GCAGTCGCCATGatgatcctgacgacgga | |
| | gaccgccgtcgtcgacaagccCGGGCGGC | |
| | Gaacatgaggatcacccatgtc | |

HEK293FT cells (American Type Culture Collection (ATCC)-CRL32156) were cultured in Dulbecco's Modified Eagle Medium with high glucose, sodium pyruvate, and GlutaMAX (Thermo Fisher Scientific), additionally supplemented with 10% (v/v) fetal bovine serum (FBS) and 1× penicillin-streptomycin (Thermo Fisher Scientific).

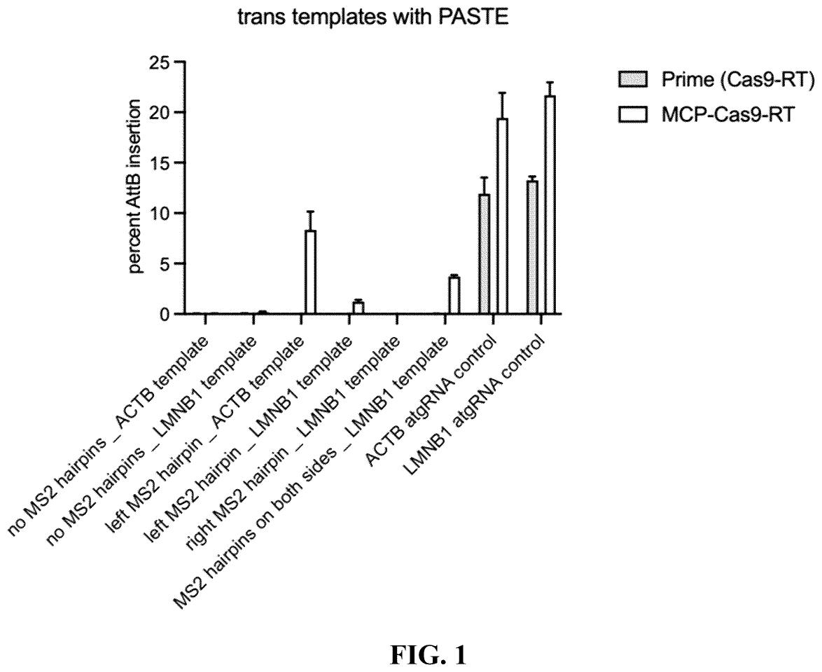

The HEK293FT cells were transfected with the ttRNAs described above (as indicated in FIG. 1) along with a Prime expression plasmid encoding a Cas9 nickase, a reverse transcriptase, a ngRNA, a targeting a gRNA targeting the LMNB1 gene or the ACTB gene; and (where indicated FIG. 1) an MS2 coat protein (MCP). Briefly, the HEK293FT cells were plated at 5-15K the day prior to transfection in a 96-well plate coated with poly-D-lysine (BD Biocoat). The transfection was subsequently carried out using with Lipofectamine 3000 (Thermo Fisher Scientific), according to the manufacturer's specifications. For insertions, of a plasmid encoding the ttRNA, 35.5 ng of a plasmid encoding the targeting gRNA, ng of a plasmid encoding the nicking gRNA, and 100 ng of a plasmid encoding the SpCas9-RT or MCP-SpCas9-RT were added to each well. The nucleic acid sequence encoding the SpCas9-RT and the MCP-SpCas9-RT editing polypeptides is provided in Table 14.

TABLE 14
Nucleic acid encoding the SpCas9-RT and the MCP-SpCas9-RT editing
polypeptides.

| Description | Nucleic Acid Sequence | SEQ ID NO |
|---|---|---|
| SpCas9-RT | atgaaacggacagccgacggaagcgagttcgagtcac | 264 |
| | caaagaagaagcggaaagtcgacaagaagtacagcat | |
| | cggcctggacatcggcaccaactctgtgggctgggcc | |
| | gtgatcaccgacgagtacaaggtgcccagcaagaaat | |
| | tcaaggtgctgggcaacaccgaccggcacagcatcaa | |
| | gaagaacctgatcggagccctgctgttcgacagcggc | |
| | gaaacagccgaggccacccggctgaagagaaccgcca | |
| | gaagaagatacaccagacggaagaaccggatctgcta | |
| | tctgcaagagatcttcagcaacgagatggccaaggtg | |
| | gacgacagcttcttccacagactggaagagtccttcc | |
| | tggtggaagaggataagaagcacgagcggcaccccat | |
| | cttcggcaacatcgtggacgaggtggcctaccacgag | |
| | aagtaccccaccatctaccacctgagaaagaaactgg | |
| | tggacagcaccgacaaggccgacctgcggctgatcta | |
| | tctggccctggcccacatgatcaagttccggggccac | |
| | ttcctgatcgagggcgacctgaaccccgacaacagcg | |
| | acgtggacaagctgttcatccagctggtgcagaccta | |
| | caaccagctgttcgaggaaaaccccatcaacgccagc | |
| | ggcgtggacgccaaggccatcctgtctgccagactga | |
| | gcaagagcagacggctggaaaatctgatcgcccagct | |
| | gcccggcgagaagaagaatggcctgttcggaaacctg | |
| | attgccctgagcctgggcctgacccccaacttcaaga | |
| | gcaacttcgacctggccgaggatgccaaactgcagct | |
| | gagcaaggacacctacgacgacgacctggacaacctg | |
| | ctggcccagatcggcgaccagtacgccgacctgtttc | |
| | tggccgccaagaacctgtccgacgccatcctgctgag | |
| | cgacatcctgagagtgaacaccgagatcaccaaggcc | |
| | cccctgagcgcctctatgatcaagagatacgacgagc | |
| | accaccaggacctgaccctgctgaaagctctcgtgcg | |
| | gcagcagctgcctgagaagtacaaagagattttcttc | |
| | gaccagagcaagaacggctacgccggctacattgacg | |
| | gcggagccagccaggaagagttctacaagttcatcaa | |
| | gcccatcctggaaaagatggacggcaccgaggaactg | |
| | ctcgtgaagctgaacagagaggacctgctgcggaagc | |
| | agcggaccttcgacaacggcagcatcccccaccagat | |
| | ccacctgggagagctgcacgccattctgcggcggcag | |
| | gaagatttttacccattcctgaaggacaaccgggaaa | |
| | agatcgagaagatcctgaccttccgcatcccctacta | |
| | cgtgggccctctggccaggggaaacagcagattcgcc | |
| | tggatgaccagaaagagcgaggaaaccatcaccccct | |
| | ggaacttcgaggaagtggtggacaagggcgcttccgc | |
| | ccagagcttcatcgagcggatgaccaacttcgataag | |
| | aacctgcccaacgagaaggtgctgcccaagcacagcc | |
| | tgctgtacgagtacttcaccgtgtataacgagctgac | |
| | caaagtgaaatacgtgaccgagggaatgagaaagccc | |
| | gccttcctgagcggcgagcagaaaaaggccatcgtgg | |
| | acctgctgttcaagaccaaccggaaagtgaccgtgaa | |
| | gcagctgaaagaggactacttcaagaaaatcgagtgc | |
| | ttcgactccgtggaaatctccggcgtggaagatcggt | |
| | tcaacgcctccctgggcacataccacgatctgctgaa | |
| | aattatcaaggacaaggacttcctggacaatgaggaa | |
| | aacgaggacattctggaagatatcgtgctgaccctga | |
| | cactgtttgaggacagagagatgatcgaggaacggct | |
| | gaaaacctatgcccacctgttcgacgacaaagtgatg | |
| | aagcagctgaagcggcggagatacaccggctggggca | |
| | ggctgagccggaagctgatcaacggcatccgggacaa | |
| | gcagtccggcaagacaatcctggatttcctgaagtcc | |
| | gacggcttcgccaacagaaacttcatgcagctgatcc | |
| | acgacgacagcctgacctttaaagaggacatccagaa | |
| | agcccaggtgtccggccagggcgatagcctgcacgag | |
| | cacattgccaatctggccggcagccccgccattaaga | |
| | agggcatcctgcagacagtgaaggtggtggacgagct | |
| | cgtgaaagtgatgggccggcacaagcccgagaacatc | |
| | gtgatcgaaatggccagagagaaccagaccacccaga | |
| | agggacagaagaacagccgcgagagaatgaagcggat | |
| | cgaagagggcatcaaagagctgggcagccagatcctg | |
| | aaagaacaccccgtggaaaacacccagctgcagaacg | |
| | agaagctgtacctgtactacctgcagaatgggcggga | |
| | tatgtacgtggaccaggaactggacatcaaccggctg | |
| | tccgactacgatgtggacgctatcgtgcctcagagct | |
| | ttctgaaggacgactccatcgacaacaaggtgctgac | |
| | cagaagcgacaagaaccggggcaagagcgacaacgtg | |
| | ccctccgaagaggtcgtgaagaagatgaagaactact | |
| | ggcggcagctgctgaacgccaagctgattacccagag | |
| | aaagttcgacaatctgaccaaggccgagagaggcggc | |
| | ctgagcgaactggataaggccggcttcatcaagagac | |
| | agctggtggaaacccggcagatcacaaagcacgtggc | |
| | acagatcctggactcccggatgaacactaagtacgac | |
| | gagaatgacaagctgatccgggaagtgaaagtgatca | |
| | ccctgaagtccaagctggtgtccgatttccggaagga | |
| | tttccagttttacaaagtgcgcgagatcaacaactac | |
| | caccacgcccacgacgcctacctgaacgccgtcgtgg | |
| | gaaccgccctgatcaaaaagtaccctaagctggaaag | |
| | cgagttcgtgtacggcgactacaaggtgtacgacgtg | |
| | cggaagatgatcgccaagagcgagcaggaaatcggca | |
| | aggctaccgccaagtacttcttctacagcaacatcat | |
| | gaactttttcaagaccgagattaccctggccaacggc | |
| | gagatccggaagcggcctctgatcgagacaaacggcg | |
| | aaaccggggagatcgtgtgggataagggccgggattt | |
| | tgccaccgtgcggaaagtgctgagcatgccccaagtg | |
| | aatatcgtgaaaaagaccgaggtgcagacaggcggct | |
| | tcagcaaagagtctatcctgcccaagaggaacagcga | |
| | taagctgatcgccagaaagaaggactgggaccctaag | |
| | aagtacggcggcttcgacagccccaccgtggcctatt | |
| | ctgtgctggtggtggccaaagtggaaaagggcaagtc | |
| | caagaaactgaagagtgtgaaagagctgctggggatc | |
| | accatcatggaaagaagcagcttcgagaagaatccca | |
| | tcgactttctggaagccaagggctacaaagaagtgaa | |
| | aaaggacctgatcatcaagctgcctaagtactccctg | |
| | ttcgagctggaaaacggccggaagagaatgctggcct | |
| | ctgccggcgaactgcagaagggaaacgaactggccct | |
| | gccctccaaatatgtgaacttcctgtacctggccagc | |
| | cactatgagaagctgaagggctcccccgaggataatg | |
| | agcagaaacagctgtttgtggaacagcacaagcacta | |
| | cctggacgagatcatcgagcagatcagcgagttctcc | |
| | aagagagtgatcctggccgacgctaatctggacaaag | |
| | tgctgtccgcctacaacaagcaccgggataagcccat | |
| | cagagagcaggccgagaatatcatccacctgtttacc | |
| | ctgaccaatctgggagcccctgccgccttcaagtact | |
| | ttgacaccaccatcgaccggaagaggtacaccagcac | |
| | caaagaggtgctggacgccaccctgatccaccagagc | |
| | atcaccggcctgtacgagacacggatcgacctgtctc | |
| | agctgggaggtgactctggaggatctagcggaggatc | |
| | ctctggcagcgagacaccaggaacaagcgagtcagca | |
| | acaccagagagcagtggcggcagcagcggcggcagca | |
| | gcaccctaaatatagaagatgagtatcggctacatga | |
| | gacctcaaaagagccagatgtttctctagggtccaca | |
| | tggctgtctgattttcctcaggcctgggcggaaaccg | |
| | ggggcatgggactggcagttcgccaagctcctctgat | |
| | catacctctgaaagcaacctctacccccgtgtccata | |
| | aaacaataccccatgtcacaagaagccagactgggga | |
| | tcaagccccacatacagagactgttggaccagggaat | |
| | actggtaccctgccagtccccctggaacacgcccctg | |
| | ctacccgttaagaaaccagggactaatgattataggc | |
| | ctgtccaggatctgagagaagtcaacaagcgggtgga | |
| | agacatccaccccaccgtgcccaaccettacaacctc | |
| | ttgagcgggctcccaccgtcccaccagtggtacactg | |
| | tgcttgatttaaaggatgcctttttctgcctgagact | |
| | ccaccccaccagtcagcctctcttcgcctttgagtgg | |
| | agagatccagagatgggaatctcaggacaattgacct | |
| | ggaccagactcccacagggtttcaaaaacagtcccac | |
| | cctgtttaatgaggcactgcacagagacctagcagac | |
| | ttccggatccagcacccagacttgatcctgctacagt | |
| | acgtggatgacttactgctggccgccacttctgagct | |
| | agactgccaacaaggtactcgggccctgttacaaacc | |
| | ctagggaacctcgggtatcgggcctcggccaagaaag | |
| | cccaaatttgccagaaacaggtcaagtatctggggta | |
| | tcttctaaaagagggtcagagatggctgactgaggcc | |
| | agaaaagagactgtgatggggcagcctactccgaaga | |
| | cccctcgacaactaagggagttcctagggaaggcagg | |
| | cttctgtcgcctcttcatccctgggtttgcagaaatg | |
| | gcagcccccctgtaccctctcaccaaaccggggactc | |
| | tgtttaattggggcccagaccaacaaaaggcctatca | |
| | agaaatcaagcaagctcttctaactgccccagccctg | |
| | gggttgccagatttgactaagccctttgaactctttg | |
| | tcgacgagaagcagggctacgccaaaggtgtcctaac | |
| | gcaaaaactgggaccttggcgtcggccggtggcctac | |
| | ctgtccaaaaagctagacccagtagcagctgggtggc | |
| | ccccttgcctacggatggtagcagccattgccgtact | |
| | gacaaaggatgcaggcaagctaaccatgggacagcca | |
| | ctagtcattctggccccccatgcagtagaggcactag | |
| | tcaaacaaccccccgaccgctggctttccaacgcccg | |
| | gatgactcactatcaggccttgcttttggacacggac | |
| | cgggtccagttcggaccggtggtagccctgaacccgg | |
| | ctacgctgctcccactgcctgaggaagggctgcaaca | |
| | caactgccttgatatcctggccgaagcccacggaacc | |
| | cgacccgacctaacggaccagccgctcccagacgccg | |
| | accacacctggtacacggatggaagcagtctcttaca | |
| | agagggacagcgtaaggcgggagctgcggtgaccacc | |
| | gagaccgaggtaatctgggctaaagccctgccagccg | |
| | ggacatccgctcagcgggctgaactgatagcactcac | |
| | ccaggccctaaagatggcagaaggtaagaagctaaat | |
| | gtttatactgatagccgttatgcttttgctactgccc | |
| | atatccatggagaaatatacagaaggcgtgggtggct | |
| | cacatcagaaggcaaagagatcaaaaataaagacgag | |
| | atcttggccctactaaaagccctctttctgcccaaaa | |
| | gacttagcataatccattgtccaggacatcaaaaggg | |
| | acacagcgccgaggctagaggcaaccggatggctgac | |
| | caagcggcccgaaaggcagccatcacagagactccag | |
| | acacctctaccctcctcatagaaaattcatcaccc | |
| MCP-Cas9-RT | atggcttcaaactttactcagttcgtgctcgtggaca | 265 |
| | atggtgggacaggggatgtgacagtggctccttctaa | |
| | tttcgctaatggggtggcagagtggatcagctccaac | |
| | tcacggagccaggcctacaaggtgacatgcagcgtca | |
| | ggcagtctagtgcccagaagagaaagtataccatcaa | |
| | ggtggaggtccccaaagtggctacccagacagtgggc | |
| | ggagtcgaactgcctgtcgccgcttggaggtcctacc | |
| | tgaacatggagctcactatcccaattttcgctaccaa | |
| | ttctgactgtgaactcatcgtgaaggcaatgcagggg | |
| | ctcctcaaagacggtaatcctatcccttccgccatcg | |
| | ccgctaactcaggtatctacagcgctggaggaggtgg | |
| | aagcggaggaggaggaagcggaggaggaggtagcgga | |
| | atgaaacggacagccgacggaagcgagttcgagtcac | |
| | caaagaagaagcggaaagtcgacaagaagtacagcat | |
| | cggcctggacatcggcaccaactctgtgggctgggcc | |
| | gtgatcaccgacgagtacaaggtgcccagcaagaaat | |
| | tcaaggtgctgggcaacaccgaccggcacagcatcaa | |
| | gaagaacctgatcggagccctgctgttcgacagcggc | |
| | gaaacagccgaggccacccggctgaagagaaccgcca | |
| | gaagaagatacaccagacggaagaaccggatctgcta | |
| | tctgcaagagatcttcagcaacgagatggccaaggtg | |
| | gacgacagcttcttccacagactggaagagtccttcc | |
| | tggtggaagaggataagaagcacgagcggcaccccat | |
| | cttcggcaacatcgtggacgaggtggcctaccacgag | |
| | aagtaccccaccatctaccacctgagaaagaaactgg | |
| | tggacagcaccgacaaggccgacctgcggctgatcta | |
| | tctggccctggcccacatgatcaagttccggggccac | |
| | ttcctgatcgagggcgacctgaaccccgacaacagcg | |
| | acgtggacaagctgttcatccagctggtgcagaccta | |
| | caaccagctgttcgaggaaaaccccatcaacgccagc | |
| | ggcgtggacgccaaggccatcctgtctgccagactga | |
| | gcaagagcagacggctggaaaatctgatcgcccagct | |
| | gcccggcgagaagaagaatggcctgttcggaaacctg | |
| | attgccctgagcctgggcctgacccccaacttcaaga | |
| | gcaacttcgacctggccgaggatgccaaactgcagct | |
| | gagcaaggacacctacgacgacgacctggacaacctg | |
| | ctggcccagatcggcgaccagtacgccgacctgtttc | |
| | tggccgccaagaacctgtccgacgccatcctgctgag | |
| | cgacatcctgagagtgaacaccgagatcaccaaggcc | |
| | cccctgagcgcctctatgatcaagagatacgacgagc | |
| | accaccaggacctgaccctgctgaaagctctcgtgcg | |
| | gcagcagctgcctgagaagtacaaagagattttcttc | |
| | gaccagagcaagaacggctacgccggctacattgacg | |
| | gcggagccagccaggaagagttctacaagttcatcaa | |
| | gcccatcctggaaaagatggacggcaccgaggaactg | |
| | ctcgtgaagctgaacagagaggacctgctgcggaagc | |
| | agcggaccttcgacaacggcagcatcccccaccagat | |
| | ccacctgggagagctgcacgccattctgcggcggcag | |
| | gaagatttttacccattcctgaaggacaaccgggaaa | |
| | agatcgagaagatcctgaccttccgcatcccctacta | |
| | cgtgggccctctggccaggggaaacagcagattcgcc | |
| | tggatgaccagaaagagcgaggaaaccatcaccccct | |
| | ggaacttcgaggaagtggtggacaagggcgcttccgc | |
| | ccagagcttcatcgagcggatgaccaacttcgataag | |
| | aacctgcccaacgagaaggtgctgcccaagcacagcc | |
| | tgctgtacgagtacttcaccgtgtataacgagctgac | |
| | caaagtgaaatacgtgaccgagggaatgagaaagccc | |
| | gccttcctgagcggcgagcagaaaaaggccatcgtgg | |
| | acctgctgttcaagaccaaccggaaagtgaccgtgaa | |
| | gcagctgaaagaggactacttcaagaaaatcgagtgc | |
| | ttcgactccgtggaaatctccggcgtggaagatcggt | |
| | tcaacgcctccctgggcacataccacgatctgctgaa | |
| | aattatcaaggacaaggacttcctggacaatgaggaa | |
| | aacgaggacattctggaagatatcgtgctgaccctga | |
| | cactgtttgaggacagagagatgatcgaggaacggct | |
| | gaaaacctatgcccacctgttcgacgacaaagtgatg | |
| | aagcagctgaagcggcggagatacaccggctggggca | |
| | ggctgagccggaagctgatcaacggcatccgggacaa | |
| | gcagtccggcaagacaatcctggatttcctgaagtcc | |
| | gacggcttcgccaacagaaacttcatgcagctgatcc | |
| | acgacgacagcctgacctttaaagaggacatccagaa | |
| | agcccaggtgtccggccagggcgatagcctgcacgag | |
| | cacattgccaatctggccggcagccccgccattaaga | |
| | agggcatcctgcagacagtgaaggtggtggacgagct | |
| | cgtgaaagtgatgggccggcacaagcccgagaacatc | |
| | gtgatcgaaatggccagagagaaccagaccacccaga | |
| | agggacagaagaacagccgcgagagaatgaagcggat | |
| | cgaagagggcatcaaagagctgggcagccagatcctg | |
| | aaagaacaccccgtggaaaacacccagctgcagaacg | |
| | agaagctgtacctgtactacctgcagaatgggcggga | |
| | tatgtacgtggaccaggaactggacatcaaccggctg | |
| | tccgactacgatgtggacgctatcgtgcctcagagct | |
| | ttctgaaggacgactccatcgacaacaaggtgctgac | |
| | cagaagcgacaagaaccggggcaagagcgacaacgtg | |
| | ccctccgaagaggtcgtgaagaagatgaagaactact | |
| | ggcggcagctgctgaacgccaagctgattacccagag | |
| | aaagttcgacaatctgaccaaggccgagagaggcggc | |
| | ctgagcgaactggataaggccggcttcatcaagagac | |
| | agctggtggaaacccggcagatcacaaagcacgtggc | |
| | acagatcctggactcccggatgaacactaagtacgac | |
| | gagaatgacaagctgatccgggaagtgaaagtgatca | |
| | ccctgaagtccaagctggtgtccgatttccggaagga | |
| | tttccagttttacaaagtgcgcgagatcaacaactac | |
| | caccacgcccacgacgcctacctgaacgccgtcgtgg | |
| | gaaccgccctgatcaaaaagtaccctaagctggaaag | |
| | cgagttcgtgtacggcgactacaaggtgtacgacgtg | |
| | cggaagatgatcgccaagagcgagcaggaaatcggca | |
| | aggctaccgccaagtacttcttctacagcaacatcat | |
| | gaactttttcaagaccgagattaccctggccaacggc | |
| | gagatccggaagcggcctctgatcgagacaaacggcg | |
| | aaaccggggagatcgtgtgggataagggccgggattt | |
| | tgccaccgtgcggaaagtgctgagcatgccccaagtg | |
| | aatatcgtgaaaaagaccgaggtgcagacaggcggct | |
| | tcagcaaagagtctatcctgcccaagaggaacagcga | |
| | taagctgatcgccagaaagaaggactgggaccctaag | |
| | aagtacggcggcttcgacagccccaccgtggcctatt | |
| | ctgtgctggtggtggccaaagtggaaaagggcaagtc | |
| | caagaaactgaagagtgtgaaagagctgctggggatc | |
| | accatcatggaaagaagcagcttcgagaagaatccca | |
| | tcgactttctggaagccaagggctacaaagaagtgaa | |
| | aaaggacctgatcatcaagctgcctaagtactccctg | |
| | ttcgagctggaaaacggccggaagagaatgctggcct | |
| | ctgccggcgaactgcagaagggaaacgaactggccct | |
| | gccctccaaatatgtgaacttcctgtacctggccagc | |
| | cactatgagaagctgaagggctcccccgaggataatg | |
| | agcagaaacagctgtttgtggaacagcacaagcacta | |
| | cctggacgagatcatcgagcagatcagcgagttctcc | |
| | aagagagtgatcctggccgacgctaatctggacaaag | |
| | tgctgtccgcctacaacaagcaccgggataagcccat | |
| | cagagagcaggccgagaatatcatccacctgtttacc | |
| | ctgaccaatctgggagcccctgccgccttcaagtact | |
| | ttgacaccaccatcgaccggaagaggtacaccagcac | |
| | caaagaggtgctggacgccaccctgatccaccagagc | |
| | atcaccggcctgtacgagacacggatcgacctgtctc | |
| | agctgggaggtgactctggaggatctagcggaggatc | |
| | ctctggcagcgagacaccaggaacaagcgagtcagca | |
| | acaccagagagcagtggcggcagcagcggcggcagca | |
| | gcaccctaaatatagaagatgagtatcggctacatga | |
| | gacctcaaaagagccagatgtttctctagggtccaca | |
| | tggctgtctgattttcctcaggcctgggcggaaaccg | |
| | ggggcatgggactggcagttcgccaagctcctctgat | |
| | catacctctgaaagcaacctctacccccgtgtccata | |
| | aaacaataccccatgtcacaagaagccagactgggga | |
| | tcaagccccacatacagagactgttggaccagggaat | |
| | actggtaccctgccagtccccctggaacacgcccctg | |
| | ctacccgttaagaaaccagggactaatgattataggc | |
| | ctgtccaggatctgagagaagtcaacaagcgggtgga | |
| | agacatccaccccaccgtgcccaaccettacaacctc | |
| | ttgagcgggctcccaccgtcccaccagtggtacactg | |
| | tgcttgatttaaaggatgcctttttctgcctgagact | |
| | ccaccccaccagtcagcctctcttcgcctttgagtgg | |
| | agagatccagagatgggaatctcaggacaattgacct | |
| | ggaccagactcccacagggtttcaaaaacagtcccac | |
| | cctgtttaatgaggcactgcacagagacctagcagac | |
| | ttccggatccagcacccagacttgatcctgctacagt | |
| | acgtggatgacttactgctggccgccacttctgagct | |
| | agactgccaacaaggtactcgggccctgttacaaacc | |
| | ctagggaacctcgggtatcgggcctcggccaagaaag | |
| | cccaaatttgccagaaacaggtcaagtatctggggta | |
| | tcttctaaaagagggtcagagatggctgactgaggcc | |
| | agaaaagagactgtgatggggcagcctactccgaaga | |
| | cccctcgacaactaagggagttcctagggaaggcagg | |
| | cttctgtcgcctcttcatccctgggtttgcagaaatg | |
| | gcagcccccctgtaccctctcaccaaaccggggactc | |
| | tgtttaattggggcccagaccaacaaaaggcctatca | |
| | agaaatcaagcaagctcttctaactgccccagccctg | |
| | gggttgccagatttgactaagccctttgaactctttg | |
| | tcgacgagaagcagggctacgccaaaggtgtcctaac | |
| | gcaaaaactgggaccttggcgtcggccggtggcctac | |
| | ctgtccaaaaagctagacccagtagcagctgggtggc | |
| | ccccttgcctacggatggtagcagccattgccgtact | |
| | gacaaaggatgcaggcaagctaaccatgggacagcca | |
| | ctagtcattctggccccccatgcagtagaggcactag | |
| | tcaaacaaccccccgaccgctggctttccaacgcccg | |
| | gatgactcactatcaggccttgcttttggacacggac | |
| | cgggtccagttcggaccggtggtagccctgaacccgg | |
| | ctacgctgctcccactgcctgaggaagggctgcaaca | |
| | caactgccttgatatcctggccgaagcccacggaacc | |
| | cgacccgacctaacggaccagccgctcccagacgccg | |
| | accacacctggtacacggatggaagcagtctcttaca | |
| | agagggacagcgtaaggcgggagctgcggtgaccacc | |
| | gagaccgaggtaatctgggctaaagccctgccagccg | |
| | ggacatccgctcagcgggctgaactgatagcactcac | |
| | ccaggccctaaagatggcagaaggtaagaagctaaat | |
| | gtttatactgatagccgttatgcttttgctactgccc | |
| | atatccatggagaaatatacagaaggcgtgggyggct | |
| | cacatcagaaggcaaagagatcaaaaataaagacgag | |
| | atcttggccctactaaaagccctctttctgcccaaaa | |
| | gacttagcataatccattgtccaggacatcaaaaggg | |
| | acacagcgccgaggctagaggcaaccggatggctgac | |
| | caagcggcccgaaaggcagccatcacagagactccag | |
| | acacctctaccctcctcatagaaaattcatcaccc | |

The nucleic acid sequence of the additional gRNAs referenced above is provided in Table 15.

TABLE 15
Nucleic acid sequence of non-ttRNA gRNAs.

| Description | Nucleic Acid Sequence | SEQ ID NO |
|---|---|---|
| ACTB N-term | GAAGCCGGCCTTGCACATGCgttttagagctagaaat | 266 |
| Nicking guide 1 | agcaagttaaaataaggctagtccgttatcaacttga | |
| +48 guide | aaaagtggcaccgagtcggtgc | |
| ACTB N-term | GCTATTCTCGCAGCTCACCAgttttagagctagaaat | 267 |
| site cutting | agcaagttaaaataaggctagtccgttatcaacttga | |
| guide | aaaagtggcaccgagtcggtgc | |
| LMNB1 N-term | GCGTGGTGGGGCCGCCAGCGgttttagagctagaaat | 268 |
| Nicking guide 1 | agcaagttaaaataaggctagtccgttatcaacttga | |
| +46 | aaaagtggcaccgagtcggtgc | |
| LMNB1 N-term | GCTGTCTCCGCCGCCCGCCAgttttagagctagaaat | 269 |
| atg site cutting | agcaagttaaaataaggctagtccgttatcaacttga | |
| guide | aaaagtggcaccgagtcggtgc | |

Integration of the integration recognition site was determined by genomic DNA harvesting and targeted amplicon next generation sequencing. Briefly, DNA was harvested from transfected cells by removal of the culture media, resuspension of the cells in 50 μL of QuickExtract (Lucigen), and incubation at 65° C. for 15 min, 68° C. for 15 min, and 98° C. for 10 min. Target genomic regions were PCR amplified with NEBNext High-Fidelity 2×PCR Master Mix (NEB) according to the manufacturer's protocol. Barcodes and adapters for Illumina sequencing were added in a subsequent PCR amplification. Amplicons were pooled and prepared for sequencing on a MiSeq (Illumina). Reads were demultiplexed and analyzed with appropriate pipelines.

As shown in FIG. 1, use of a separate ttRNA and target gRNA within the PASTE editing system mediates integration of integration recognition sites at two different target sites (LMNB1 and ACTB). The integration required the addition of an aptamer binding protein to the editing polypeptide to recruit the ttRNA with the corresponding aptamer to the target site. This example shows that the classical pegRNA utilized in PRIME editing, can be split into two molecules, a targeting gRNA targeting the Cas9 nickase to the target site and a ttRNA comprising the primer binding site, the reverse transcriptase template sequence, and the integration sequence, while maintaining efficient PASTE integration.

### 9.2 Example 2. Programmable Addition Via Site Specific Targeting Elements (PASTE) with Trans-Template RNA (ttRNA)

The ttRNA generated in Example 1 (see Table 13) are used to incorporate an integration recognition site into the target sites in the ACTB and LMNB1 loci and then integrate an exogenous polynucleotide of interest using the integration recognition site as a landing pad or beacon. In this example, incorporation of the integration recognition site and the exogenous polynucleotide of interest is mediated by an MCP-Cas9-RT polypeptide and a Bxb1 integrase.

HEK293FT cells (American Type Culture Collection (ATCC)-CRL32156) are cultured in Dulbecco's Modified Eagle Medium with high glucose, sodium pyruvate, and GlutaMAX (Thermo Fisher Scientific), additionally supplemented with 10% (v/v) fetal bovine serum (FBS) and 1× penicillin-streptomycin (Thermo Fisher Scientific).

The HEK293FT cells are transfected with the ttRNAs described above in Example 1 (as indicated in FIG. 1; see Table 13 for ttRNA) along with an expression plasmid (or multiple expression plasmids) encoding a Cas9 nickase, a reverse transcriptase fusion with MS2 coating protein, a ngRNA, a targeting a gRNA targeting the LMNB1 gene or the ACTB gene; and (where indicated FIG. 1) and a Bxb1 integrase. Briefly, the HEK293FT cells are plated at 5-15K cells per well the day prior to transfection in a 96-well plate coated with poly-D-lysine (BD Biocoat). The transfection is carried out using with Lipofectamine 3000 (Thermo Fisher Scientific), according to the manufacturer's specifications. For insertions, 35.5 ng of a plasmid encoding the ttRNA, 35.5 ng of a plasmid encoding the targeting gRNA, 25 ng of a plasmid encoding the nicking gRNA, 20 ng of plasmid encoding the exogenous polynucleotide, and 100 ng of a plasmid encoding the SpCas9-RT; SpCas-RT-Bxb1, MCP-SpCas9-RT, or MCP-SpCas9-RT-Bxb1 are added to each well. The nucleic acid sequence encoding the SpCas9-RT and the MCP-SpCas9-RT editing polypeptides is provided in Table 14 (see Example 1) and the nucleic acid sequence encoding the SpCas-RT-Bxb1 and MCP-SpCas9-RT-Bxb1 is provided in Table 16.

TABLE 16
nucleic acid sequence encoding the SpCas-RT-Bxb1 and MCP-SpCas9-RT-Bxb1.

| | | SEQ |
|---|---|---|
| | | ID |
| Description | Nucleic Acid Sequence | NO: |
| SpCas-RT-Bxb1 | ATGAAACGGACAGCCGACGGAAGCGAGTTCGAGTCACCAAAGAAGA | 270 |
| | AGCGGAAAGTCGACAAGAAGTACAGCATCGGCCTGGACATCGGCAC | |
| | CAACTCTGTGGGCTGGGCCGTGATCACCGACGAGTACAAGGTGCCCA | |
| | GCAAGAAATTCAAGGTGCTGGGCAACACCGACCGGCACAGCATCAA | |
| | GAAGAACCTGATCGGAGCCCTGCTGTTCGACAGCGGCGAAACAGCC | |
| | GAGGCCACCCGGCTGAAGAGAACCGCCAGAAGAAGATACACCAGAC | |
| | GGAAGAACCGGATCTGCTATCTGCAAGAGATCTTCAGCAACGAGAT | |
| | GGCCAAGGTGGACGACAGCTTCTTCCACAGACTGGAAGAGTCCTTCC | |
| | TGGTGGAAGAGGATAAGAAGCACGAGCGGCACCCCATCTTCGGCAA | |
| | CATCGTGGACGAGGTGGCCTACCACGAGAAGTACCCCACCATCTACC | |
| | ACCTGAGAAAGAAACTGGTGGACAGCACCGACAAGGCCGACCTGCG | |
| | GCTGATCTATCTGGCCCTGGCCCACATGATCAAGTTCCGGGGCCACT | |
| | TCCTGATCGAGGGCGACCTGAACCCCGACAACAGCGACGTGGACAAG | |
| | CTGTTCATCCAGCTGGTGCAGACCTACAACCAGCTGTTCGAGGAAAA | |
| | CCCCATCAACGCCAGCGGCGTGGACGCCAAGGCCATCCTGTCTGCCA | |
| | GACTGAGCAAGAGCAGACGGCTGGAAAATCTGATCGCCCAGCTGCC | |
| | CGGCGAGAAGAAGAATGGCCTGTTCGGAAACCTGATTGCCCTGAGC | |
| | CTGGGCCTGACCCCCAACTTCAAGAGCAACTTCGACCTGGCCGAGGA | |
| | TGCCAAACTGCAGCTGAGCAAGGACACCTACGACGACGACCTGGAC | |
| | AACCTGCTGGCCCAGATCGGCGACCAGTACGCCGACCTGTTTCTGGC | |
| | CGCCAAGAACCTGTCCGACGCCATCCTGCTGAGCGACATCCTGAGAG | |
| | TGAACACCGAGATCACCAAGGCCCCCCTGAGCGCCTCTATGATCAAG | |
| | AGATACGACGAGCACCACCAGGACCTGACCCTGCTGAAAGCTCTCGT | |
| | GCGGCAGCAGCTGCCTGAGAAGTACAAAGAGATTTTCTTCGACCAG | |
| | AGCAAGAACGGCTACGCCGGCTACATTGACGGCGGAGCCAGCCAGG | |
| | AAGAGTTCTACAAGTTCATCAAGCCCATCCTGGAAAAGATGGACGG | |
| | CACCGAGGAACTGCTCGTGAAGCTGAACAGAGAGGACCTGCTGCGG | |
| | AAGCAGCGGACCTTCGACAACGGCAGCATCCCCCACCAGATCCACCT | |
| | GGGAGAGCTGCACGCCATTCTGCGGCGGCAGGAAGATTTTTACCCA | |
| | TTCCTGAAGGACAACCGGGAAAAGATCGAGAAGATCCTGACCTTCC | |
| | GCATCCCCTACTACGTGGGCCCTCTGGCCAGGGGAAACAGCAGATTC | |
| | GCCTGGATGACCAGAAAGAGCGAGGAAACCATCACCCCCTGGAACT | |
| | TCGAGGAAGTGGTGGACAAGGGCGCTTCCGCCCAGAGCTTCATCGA | |
| | GCGGATGACCAACTTCGATAAGAACCTGCCCAACGAGAAGGTGCTG | |
| | CCCAAGCACAGCCTGCTGTACGAGTACTTCACCGTGTATAACGAGCT | |
| | GACCAAAGTGAAATACGTGACCGAGGGAATGAGAAAGCCCGCCTTC | |
| | CTGAGCGGCGAGCAGAAAAAGGCCATCGTGGACCTGCTGTTCAAGA | |
| | CCAACCGGAAAGTGACCGTGAAGCAGCTGAAAGAGGACTACTTCAA | |
| | GAAAATCGAGTGCTTCGACTCCGTGGAAATCTCCGGCGTGGAAGAT | |
| | CGGTTCAACGCCTCCCTGGGCACATACCACGATCTGCTGAAAATTAT | |
| | CAAGGACAAGGACTTCCTGGACAATGAGGAAAACGAGGACATTCTG | |
| | GAAGATATCGTGCTGACCCTGACACTGTTTGAGGACAGAGAGATGA | |
| | TCGAGGAACGGCTGAAAACCTATGCCCACCTGTTCGACGACAAAGT | |
| | GATGAAGCAGCTGAAGCGGCGGAGATACACCGGCTGGGGCAGGCTG | |
| | AGCCGGAAGCTGATCAACGGCATCCGGGACAAGCAGTCCGGCAAGA | |
| | CAATCCTGGATTTCCTGAAGTCCGACGGCTTCGCCAACAGAAACTTC | |
| | ATGCAGCTGATCCACGACGACAGCCTGACCTTTAAAGAGGACATCC | |
| | AGAAAGCCCAGGTGTCCGGCCAGGGCGATAGCCTGCACGAGCACAT | |
| | TGCCAATCTGGCCGGCAGCCCCGCCATTAAGAAGGGCATCCTGCAGA | |
| | CAGTGAAGGTGGTGGACGAGCTCGTGAAAGTGATGGGCCGGCACAA | |
| | GCCCGAGAACATCGTGATCGAAATGGCCAGAGAGAACCAGACCACC | |
| | CAGAAGGGACAGAAGAACAGCCGCGAGAGAATGAAGCGGATCGAAG | |
| | AGGGCATCAAAGAGCTGGGCAGCCAGATCCTGAAAGAACACCCCGT | |
| | GGAAAACACCCAGCTGCAGAACGAGAAGCTGTACCTGTACTACCTG | |
| | CAGAATGGGCGGGATATGTACGTGGACCAGGAACTGGACATCAACC | |
| | GGCTGTCCGACTACGATGTGGACGCTATCGTGCCTCAGAGCTTTCTG | |
| | AAGGACGACTCCATCGACAACAAGGTGCTGACCAGAAGCGACAAGA | |
| | ACCGGGGCAAGAGCGACAACGTGCCCTCCGAAGAGGTCGTGAAGAA | |
| | GATGAAGAACTACTGGCGGCAGCTGCTGAACGCCAAGCTGATTACC | |
| | CAGAGAAAGTTCGACAATCTGACCAAGGCCGAGAGAGGCGGCCTGA | |
| | GCGAACTGGATAAGGCCGGCTTCATCAAGAGACAGCTGGTGGAAAC | |
| | CCGGCAGATCACAAAGCACGTGGCACAGATCCTGGACTCCCGGATGA | |
| | ACACTAAGTACGACGAGAATGACAAGCTGATCCGGGAAGTGAAAGT | |
| | GATCACCCTGAAGTCCAAGCTGGTGTCCGATTTCCGGAAGGATTTCC | |
| | AGTTTTACAAAGTGCGCGAGATCAACAACTACCACCACGCCCACGAC | |
| | GCCTACCTGAACGCCGTCGTGGGAACCGCCCTGATCAAAAAGTACCC | |
| | TAAGCTGGAAAGCGAGTTCGTGTACGGCGACTACAAGGTGTACGAC | |
| | GTGCGGAAGATGATCGCCAAGAGCGAGCAGGAAATCGGCAAGGCTA | |
| | CCGCCAAGTACTTCTTCTACAGCAACATCATGAACTTTTTCAAGACC | |
| | GAGATTACCCTGGCCAACGGCGAGATCCGGAAGCGGCCTCTGATCGA | |
| | GACAAACGGCGAAACCGGGGAGATCGTGTGGGATAAGGGCCGGGAT | |
| | TTTGCCACCGTGCGGAAAGTGCTGAGCATGCCCCAAGTGAATATCG | |
| | TGAAAAAGACCGAGGTGCAGACAGGCGGCTTCAGCAAAGAGTCTAT | |
| | CCTGCCCAAGAGGAACAGCGATAAGCTGATCGCCAGAAAGAAGGAC | |
| | TGGGACCCTAAGAAGTACGGCGGCTTCGACAGCCCCACCGTGGCCTA | |
| | TTCTGTGCTGGTGGTGGCCAAAGTGGAAAAGGGCAAGTCCAAGAAA | |
| | CTGAAGAGTGTGAAAGAGCTGCTGGGGATCACCATCATGGAAAGAA | |
| | GCAGCTTCGAGAAGAATCCCATCGACTTTCTGGAAGCCAAGGGCTA | |
| | CAAAGAAGTGAAAAAGGACCTGATCATCAAGCTGCCTAAGTACTCC | |
| | CTGTTCGAGCTGGAAAACGGCCGGAAGAGAATGCTGGCCTCTGCCG | |
| | GCGAACTGCAGAAGGGAAACGAACTGGCCCTGCCCTCCAAATATGT | |
| | GAACTTCCTGTACCTGGCCAGCCACTATGAGAAGCTGAAGGGCTCCC | |
| | CCGAGGATAATGAGCAGAAACAGCTGTTTGTGGAACAGCACAAGCA | |
| | CTACCTGGACGAGATCATCGAGCAGATCAGCGAGTTCTCCAAGAGA | |
| | GTGATCCTGGCCGACGCTAATCTGGACAAAGTGCTGTCCGCCTACAA | |
| | CAAGCACCGGGATAAGCCCATCAGAGAGCAGGCCGAGAATATCATC | |
| | CACCTGTTTACCCTGACCAATCTGGGAGCCCCTGCCGCCTTCAAGTA | |
| | CTTTGACACCACCATCGACCGGAAGAGGTACACCAGCACCAAAGAG | |
| | GTGCTGGACGCCACCCTGATCCACCAGAGCATCACCGGCCTGTACGA | |
| | GACACGGATCGACCTGTCTCAGCTGGGAGGTGACTCTGGAGGATCT | |
| | AGCGGAGGATCCTCTGGCAGCGAGACACCAGGAACAAGCGAGTCAG | |
| | CAACACCAGAGAGCTCTGGTAGCGAGACACCCGGTACCAGTGAAAG | |
| | CGCCACGCCAGAAAGCAGTGGGAGTGAGACTCCGGGTACATCTGAA | |
| | TCAGCGACACCGGAATCAAGTGGCGGCAGCAGCGGCGGCAGCAGCA | |
| | CCCTAAATATAGAAGATGAGTATCGGCTACATGAGACCTCAAAAGA | |
| | GCCAGATGTTTCTCTAGGGTCCACATGGCTGTCTGATTTTCCTCAGG | |
| | CCTGGGCGGAAACCGGGGGCATGGGACTGGCAGTTCGCCAAGCTCCT | |
| | CTGATCATACCTCTGAAAGCAACCTCTACCCCCGTGTCCATAAAACA | |
| | ATACCCCATGTCACAAGAAGCCAGACTGGGGATCAAGCCCCACATAC | |
| | AGAGACTGTTGGACCAGGGAATACTGGTACCCTGCCAGTCCCCCTGG | |
| | AACACGCCCCTGCTACCCGTTAAGAAACCAGGGACTAATGATTATA | |
| | GGCCTGTCCAGGATCTGAGAGAAGTCAACAAGCGGGTGGAAGACAT | |
| | CCACCCCACCGTGCCCAACCCTTACAACCTCTTGAGCGGGCCCCCAC | |
| | CGTCCCACCAGTGGTACACTGTGCTTGATTTAAAGGATGCCTTTTTC | |
| | TGCCTGAGACTCCACCCCACCAGTCAGCCTCTCTTCGCCTTTGAGTG | |
| | GAGAGATCCAGAGATGGGAATCTCAGGACAATTGACCTGGACCAGA | |
| | CTCCCACAGGGTTTCAAAAACAGTCCCACCCTGTTTAATGAGGCACT | |
| | GCACAGAGACCTAGCAGACTTCCGGATCCAGCACCCAGACTTGATCC | |
| | TGCTACAGTACGTGGATGACTTACTGCTGGCCGCCACTTCTGAGCTA | |
| | GACTGCCAACAAGGTACTCGGGCCCTGTTACAAACCCTAGGGAACCT | |
| | CGGGTATCGGGCCTCGGCCAAGAAAGCCCAAATTTGCCAGAAACAG | |
| | GTCAAGTATCTGGGGTATCTTCTAAAAGAGGGTCAGAGATGGCTGA | |
| | CTGAGGCCAGAAAAGAGACTGTGATGGGGCAGCCTACTCCGAAGAC | |
| | CCCTCGACAACTAAGGGAGTTCCTAGGGAAGGCAGGCTTCTGTCGCC | |
| | TCTTCATCCCTGGGTTTGCAGAAATGGCAGCCCCCCTGTACCCTCTC | |
| | ACCAAACCGGGGACTCTGTTTAATTGGGGCCCAGACCAACAAAAGG | |
| | CCTATCAAGAAATCAAGCAAGCTCTTCTAACTGCCCCAGCCCTGGGG | |
| | TTGCCAGATTTGACTAAGCCCTTTGAACTCTTTGTCGACGAGAAGC | |
| | AGGGCTACGCCAAAGGTGTCCTAACGCAAAAACTGGGACCTTGGCG | |
| | TCGGCCGGTGGCCTACCTGTCCAAAAAGCTAGACCCAGTAGCAGCTG | |
| | GGTGGCCCCCTTGCCTACGGATGGTAGCAGCCATTGCCGTACTGACA | |
| | AAGGATGCAGGCAAGCTAACCATGGGACAGCCACTAGTCATTCTGG | |
| | CCCCCCATGCAGTAGAGGCACTAGTCAAACAACCCCCCGACCGCTGG | |
| | CTTTCCAACGCCCGGATGACTCACTATCAGGCCTTGCTTTTGGACAC | |
| | GGACCGGGTCCAGTTCGGACCGGTGGTAGCCCTGAACCCGGCTACGC | |
| | TGCTCCCACTGCCTGAGGAAGGGCTGCAACACAACTGCCTTGATATC | |
| | CTGGCCGAAGCCCACGGAACCCGACCCGACCTAACGGACCAGCCGCT | |
| | CCCAGACGCCGACCACACCTGGTACACGGATGGAAGCAGTCTCTTAC | |
| | AAGAGGGACAGCGTAAGGCGGGAGCTGCGGTGACCACCGAGACCGA | |
| | GGTAATCTGGGCTAAAGCCCTGCCAGCCGGGACATCCGCTCAGCGGG | |
| | CTGAACTGATAGCACTCACCCAGGCCCTAAAGATGGCAGAAGGTAA | |
| | GAAGCTAAATGTTTATACTGATAGCCGTTATGCTTTTGCTACTGCCC | |
| | ATATCCATGGAGAAATATACAGAAGGCGTGGGTGGCTCACATCAGA | |
| | AGGCAAAGAGATCAAAAATAAAGACGAGATCTTGGCCCTACTAAAA | |
| | GCCCTCTTTCTGCCCAAAAGACTTAGCATAATCCATTGTCCAGGACA | |
| | TCAAAAGGGACACAGCGCCGAGGCTAGAGGCAACCGGATGGCTGAC | |
| | CAAGCGGCCCGAAAGGCAGCCATCACAGAGACTCCAGACACCTCTAC | |
| | CCTCCTCATAGAAAATTCATCACCCTCTGGCGGCTCAAAAAGAACCG | |
| | CCGACGGCAGCGAATTCGAGCCCAAGAAGAAGAGGAAAGTCGGGGG | |
| | GTCAGGTGGATCCGGCGGAAGTGGCGGATCCGGTGGATCTGGCGGC | |
| | AGTCCAAAAAAGAAAAGAAAAGTGTATCCCTATGATGTCCCCGATT | |
| | ATGCCGGTTCAAGAGCCCTGGTCGTGATTAGACTGAGCCGAGTGAC | |
| | AGACGCCACCACAAGTCCCGAGAGACAGCTGGAATCATGCCAGCAGC | |
| | TCTGTGCTCAGCGGGGTTGGGATGTGGTCGGCGTGGCAGAGGATCT | |
| | GGACGTGAGCGGGGCCGTCGATCCATTCGACAGAAAGAGGAGGCCC | |
| | AACCTGGCAAGATGGCTCGCTTTCGAGGAACAGCCCTTTGATGTGA | |
| | TCGTCGCCTACAGAGTGGACCGGCTGACCCGCTCAATTCGACATCTC | |
| | CAGCAGCTGGTGCATTGGGCTGAGGACCACAAGAAACTGGTGGTCA | |
| | GCGCAACAGAAGCCCACTTCGATACTACCACACCTTTTGCCGCTGTG | |
| | GTCATCGCACTGATGGGCACTGTGGCCCAGATGGAGCTCGAAGCTA | |
| | TCAAGGAGCGAAACAGGAGCGCAGCCCATTTCAATATTAGGGCCGG | |
| | TAAATACAGAGGCTCCCTGCCCCCTTGGGGATATCTCCCTACCAGGG | |
| | TGGATGGGGAGTGGAGACTGGTGCCAGACCCCGTCCAGAGAGAGCG | |
| | GATTCTGGAAGTGTACCACAGAGTGGTCGATAACCACGAACCACTC | |
| | CATCTGGTGGCACACGACCTGAATAGACGCGGCGTGCTCTCTCCAAA | |
| | GGATTATTTTGCTCAGCTGCAGGGAAGAGAGCCACAGGGAAGAGAA | |
| | TGGAGTGCTACTGCACTGAAGAGATCTATGATCAGTGAGGCTATGC | |
| | TGGGTTACGCAACACTCAATGGCAAAACTGTCCGGGACGATGACGG | |
| | AGCCCCTCTGGTGAGGGCTGAGCCTATTCTCACCAGAGAGCAGCTCG | |
| | AAGCTCTGCGGGCAGAACTGGTCAAGACTAGTCGCGCCAAACCTGCC | |
| | GTGAGCACCCCAAGCCTGCTCCTGAGGGTGCTGTTCTGCGCCGTCTG | |
| | TGGAGAGCCAGCATACAAGTTTGCCGGCGGAGGGCGCAAACATCCC | |
| | CGCTATCGATGCAGGAGCATGGGGTTCCCTAAGCACTGTGGAAACG | |
| | GGACAGTGGCCATGGCTGAGTGGGACGCCTTTTGCGAGGAACAGGT | |
| | GCTGGATCTCCTGGGTGACGCTGAGCGGCTGGAAAAAGTGTGGGTG | |
| | GCAGGATCTGACTCCGCTGTGGAGCTGGCAGAAGTCAATGCCGAGC | |
| | TCGTGGATCTGACTTCCCTCATCGGATCTCCTGCATATAGAGCTGGG | |
| | TCCCCACAGAGAGAAGCTCTGGACGCACGAATTGCTGCACTCGCTGC | |
| | TAGACAGGAGGAACTGGAGGGCCTGGAGGCCAGGCCCTCTGGATGG | |
| | GAGTGGCGAGAAACCGGACAGAGGTTTGGGGATTGGTGGAGGGAGC | |
| | AGGACACCGCAGCCAAGAACACATGGCTGAGATCCATGAATGTCCG | |
| | GCTCACATTCGACGTGCGCGGTGGCCTGACTCGAACCATCGATTTTG | |
| | GCGACCTGCAGGAGTATGAACAGCACCTGAGACTGGGGTCCGTGGT | |
| | CGAAAGACTGCACACTGGGATGTCCTAG | |
| MCP-SpCas9- | ATGTTATTAATTAACGCTTCTAACTTTACTCAGTTCGTTCTCGTCGA | 271 |
| RT-Bxb1 | CAATGGCGGAACTGGCGACGTGACTGTCGCCCCAAGCAACTTCGCTA | |
| | ACGGGATCGCTGAATGGATCAGCTCTAACTCGCGTTCACAGGCTTAC | |
| | AAAGTAACCTGTAGCGTTCGTCAGAGCTCTGCGCAGAATCGCAAAT | |
| | ACACCATCAAAGTCGAGGTGCCTAAAGGCGCCTGGCGTTCGTACTTA | |
| | AATATGGAACTAACCATTCCAATTTTCGCCACGAATTCCGACTGCGA | |
| | GCTTATTGTTAAGGCAATGCAAGGTCTCCTAAAAGATGGAAACCCG | |
| | ATTCCCTCAGCAATCGCAGCAAACTCCGGCATCTACGCCATGGCCAG | |
| | CAACTTCACCCAGTTCGTGCTGGTGGACAACGGCGGCACCGGCGACG | |
| | TGACCGTGGCCCCCAGCAACTTCGCCAACGGCATCGCCGAGTGGATC | |
| | AGCAGCAACAGCAGAAGCCAGGCCTACAAGGTGACCTGCAGCGTGA | |
| | GACAGAGCAGCGCCCAGAACAGAAAGTACACCATCAAGGTGGAGGT | |
| | GCCCAAGGGCGCCTGGAGAAGCTACCTGAACATGGAGCTGACCATCC | |
| | CCATCTTCGCCACCAACAGCGACTGCGAGCTGATCGTGAAGGCCATG | |
| | CAGGGCCTGCTGAAGGACGGCAACCCCATCCCCAGCGCCATCGCCGC | |
| | CAACAGCGGCATCTACGCCGACATGAAACGGACAGCCGACGGAAGC | |
| | GAGTTCGAGTCACCAAAGAAGAAGCGGAAAGTCGACAAGAAGTACA | |
| | GCATCGGCCTGGACATCGGCACCAACTCTGTGGGCTGGGCCGTGATC | |
| | ACCGACGAGTACAAGGTGCCCAGCAAGAAATTCAAGGTGCTGGGCA | |
| | ACACCGACCGGCACAGCATCAAGAAGAACCTGATCGGAGCCCTGCTG | |
| | TTCGACAGCGGCGAAACAGCCGAGGCCACCCGGCTGAAGAGAACCGC | |
| | CAGAAGAAGATACACCAGACGGAAGAACCGGATCTGCTATCTGCAA | |
| | GAGATCTTCAGCAACGAGATGGCCAAGGTGGACGACAGCTTCTTCC | |
| | ACAGACTGGAAGAGTCCTTCCTGGTGGAAGAGGATAAGAAGCACGA | |
| | GCGGCACCCCATCTTCGGCAACATCGTGGACGAGGTGGCCTACCACG | |
| | AGAAGTACCCCACCATCTACCACCTGAGAAAGAAACTGGTGGACAG | |
| | CACCGACAAGGCCGACCTGCGGCTGATCTATCTGGCCCTGGCCCACA | |
| | TGATCAAGTTCCGGGGCCACTTCCTGATCGAGGGCGACCTGAACCCC | |
| | GACAACAGCGACGTGGACAAGCTGTTCATCCAGCTGGTGCAGACCT | |
| | ACAACCAGCTGTTCGAGGAAAACCCCATCAACGCCAGCGGCGTGGAC | |
| | GCCAAGGCCATCCTGTCTGCCAGACTGAGCAAGAGCAGACGGCTGG | |
| | AAAATCTGATCGCCCAGCTGCCCGGCGAGAAGAAGAATGGCCTGTT | |
| | CGGAAACCTGATTGCCCTGAGCCTGGGCCTGACCCCCAACTTCAAGA | |
| | GCAACTTCGACCTGGCCGAGGATGCCAAACTGCAGCTGAGCAAGGA | |
| | CACCTACGACGACGACCTGGACAACCTGCTGGCCCAGATCGGCGACC | |
| | AGTACGCCGACCTGTTTCTGGCCGCCAAGAACCTGTCCGACGCCATC | |
| | CTGCTGAGCGACATCCTGAGAGTGAACACCGAGATCACCAAGGCCCC | |
| | CCTGAGCGCCTCTATGATCAAGAGATACGACGAGCACCACCAGGACC | |
| | TGACCCTGCTGAAAGCTCTCGTGCGGCAGCAGCTGCCTGAGAAGTAC | |
| | AAAGAGATTTTCTTCGACCAGAGCAAGAACGGCTACGCCGGCTACA | |
| | TTGACGGCGGAGCCAGCCAGGAAGAGTTCTACAAGTTCATCAAGCC | |
| | CATCCTGGAAAAGATGGACGGCACCGAGGAACTGCTCGTGAAGCTG | |
| | AACAGAGAGGACCTGCTGCGGAAGCAGCGGACCTTCGACAACGGCA | |
| | GCATCCCCCACCAGATCCACCTGGGAGAGCTGCACGCCATTCTGCGG | |
| | CGGCAGGAAGATTTTTACCCATTCCTGAAGGACAACCGGGAAAAGA | |
| | TCGAGAAGATCCTGACCTTCCGCATCCCCTACTACGTGGGCCCTCTG | |
| | GCCAGGGGAAACAGCAGATTCGCCTGGATGACCAGAAAGAGCGAGG | |
| | AAACCATCACCCCCTGGAACTTCGAGGAAGTGGTGGACAAGGGCGC | |
| | TTCCGCCCAGAGCTTCATCGAGCGGATGACCAACTTCGATAAGAACC | |
| | TGCCCAACGAGAAGGTGCTGCCCAAGCACAGCCTGCTGTACGAGTAC | |
| | TTCACCGTGTATAACGAGCTGACCAAAGTGAAATACGTGACCGAGG | |
| | GAATGAGAAAGCCCGCCTTCCTGAGCGGCGAGCAGAAAAAGGCCAT | |
| | CGTGGACCTGCTGTTCAAGACCAACCGGAAAGTGACCGTGAAGCAG | |
| | CTGAAAGAGGACTACTTCAAGAAAATCGAGTGCTTCGACTCCGTGG | |
| | AAATCTCCGGCGTGGAAGATCGGTTCAACGCCTCCCTGGGCACATAC | |
| | CACGATCTGCTGAAAATTATCAAGGACAAGGACTTCCTGGACAATG | |
| | AGGAAAACGAGGACATTCTGGAAGATATCGTGCTGACCCTGACACT | |
| | GTTTGAGGACAGAGAGATGATCGAGGAACGGCTGAAAACCTATGCC | |
| | CACCTGTTCGACGACAAAGTGATGAAGCAGCTGAAGCGGCGGAGAT | |
| | ACACCGGCTGGGGCAGGCTGAGCCGGAAGCTGATCAACGGCATCCG | |
| | GGACAAGCAGTCCGGCAAGACAATCCTGGATTTCCTGAAGTCCGAC | |
| | GGCTTCGCCAACAGAAACTTCATGCAGCTGATCCACGACGACAGCCT | |
| | GACCTTTAAAGAGGACATCCAGAAAGCCCAGGTGTCCGGCCAGGGC | |
| | GATAGCCTGCACGAGCACATTGCCAATCTGGCCGGCAGCCCCGCCAT | |
| | TAAGAAGGGCATCCTGCAGACAGTGAAGGTGGTGGACGAGCTCGTG | |
| | AAAGTGATGGGCCGGCACAAGCCCGAGAACATCGTGATCGAAATGG | |
| | CCAGAGAGAACCAGACCACCCAGAAGGGACAGAAGAACAGCCGCGA | |
| | GAGAATGAAGCGGATCGAAGAGGGCATCAAAGAGCTGGGCAGCCAG | |
| | ATCCTGAAAGAACACCCCGTGGAAAACACCCAGCTGCAGAACGAGA | |
| | AGCTGTACCTGTACTACCTGCAGAATGGGCGGGATATGTACGTGGA | |
| | CCAGGAACTGGACATCAACCGGCTGTCCGACTACGATGTGGACGCTA | |
| | TCGTGCCTCAGAGCTTTCTGAAGGACGACTCCATCGACAACAAGGTG | |
| | CTGACCAGAAGCGACAAGAACCGGGGCAAGAGCGACAACGTGCCCT | |
| | CCGAAGAGGTCGTGAAGAAGATGAAGAACTACTGGCGGCAGCTGCT | |
| | GAACGCCAAGCTGATTACCCAGAGAAAGTTCGACAATCTGACCAAG | |
| | GCCGAGAGAGGCGGCCTGAGCGAACTGGATAAGGCCGGCTTCATCA | |
| | AGAGACAGCTGGTGGAAACCCGGCAGATCACAAAGCACGTGGCACA | |
| | GATCCTGGACTCCCGGATGAACACTAAGTACGACGAGAATGACAAG | |
| | CTGATCCGGGAAGTGAAAGTGATCACCCTGAAGTCCAAGCTGGTGT | |
| | CCGATTTCCGGAAGGATTTCCAGTTTTACAAAGTGCGCGAGATCAA | |
| | CAACTACCACCACGCCCACGACGCCTACCTGAACGCCGTCGTGGGAA | |
| | CCGCCCTGATCAAAAAGTACCCTAAGCTGGAAAGCGAGTTCGTGTA | |
| | CGGCGACTACAAGGTGTACGACGTGCGGAAGATGATCGCCAAGAGC | |
| | GAGCAGGAAATCGGCAAGGCTACCGCCAAGTACTTCTTCTACAGCA | |
| | ACATCATGAACTTTTTCAAGACCGAGATTACCCTGGCCAACGGCGA | |
| | GATCCGGAAGCGGCCTCTGATCGAGACAAACGGCGAAACCGGGGAG | |
| | ATCGTGTGGGATAAGGGCCGGGATTTTGCCACCGTGCGGAAAGTGC | |
| | TGAGCATGCCCCAAGTGAATATCGTGAAAAAGACCGAGGTGCAGAC | |
| | AGGCGGCTTCAGCAAAGAGTCTATCCTGCCCAAGAGGAACAGCGAT | |
| | AAGCTGATCGCCAGAAAGAAGGACTGGGACCCTAAGAAGTACGGCG | |
| | GCTTCGACAGCCCCACCGTGGCCTATTCTGTGCTGGTGGTGGCCAAA | |
| | GTGGAAAAGGGCAAGTCCAAGAAACTGAAGAGTGTGAAAGAGCTGC | |
| | TGGGGATCACCATCATGGAAAGAAGCAGCTTCGAGAAGAATCCCAT | |
| | CGACTTTCTGGAAGCCAAGGGCTACAAAGAAGTGAAAAAGGACCTG | |
| | ATCATCAAGCTGCCTAAGTACTCCCTGTTCGAGCTGGAAAACGGCCG | |
| | GAAGAGAATGCTGGCCTCTGCCGGCGAACTGCAGAAGGGAAACGAA | |
| | CTGGCCCTGCCCTCCAAATATGTGAACTTCCTGTACCTGGCCAGCCA | |
| | CTATGAGAAGCTGAAGGGCTCCCCCGAGGATAATGAGCAGAAACAG | |
| | CTGTTTGTGGAACAGCACAAGCACTACCTGGACGAGATCATCGAGC | |
| | AGATCAGCGAGTTCTCCAAGAGAGTGATCCTGGCCGACGCTAATCT | |
| | GGACAAAGTGCTGTCCGCCTACAACAAGCACCGGGATAAGCCCATCA | |
| | GAGAGCAGGCCGAGAATATCATCCACCTGTTTACCCTGACCAATCTG | |
| | GGAGCCCCTGCCGCCTTCAAGTACTTTGACACCACCATCGACCGGAA | |
| | GAGGTACACCAGCACCAAAGAGGTGCTGGACGCCACCCTGATCCACC | |
| | AGAGCATCACCGGCCTGTACGAGACACGGATCGACCTGTCTCAGCTG | |
| | GGAGGTGACTCTGGAGGATCTAGCGGAGGATCCTCTGGCAGCGAGA | |
| | CACCAGGAACAAGCGAGTCAGCAACACCAGAGAGCTCTGGTAGCGA | |
| | GACACCCGGTACCAGTGAAAGCGCCACGCCAGAAAGCAGTGGGAGT | |
| | GAGACTCCGGGTACATCTGAATCAGCGACACCGGAATCAAGTGGCG | |
| | GCAGCAGCGGCGGCAGCAGCACCCTAAATATAGAAGATGAGTATCG | |
| | GCTACATGAGACCTCAAAAGAGCCAGATGTTTCTCTAGGGTCCACA | |
| | TGGCTGTCTGATTTTCCTCAGGCCTGGGCGGAAACCGGGGGCATGGG | |
| | ACTGGCAGTTCGCCAAGCTCCTCTGATCATACCTCTGAAAGCAACCT | |
| | CTACCCCCGTGTCCATAAAACAATACCCCATGTCACAAGAAGCCAGA | |
| | CTGGGGATCAAGCCCCACATACAGAGACTGTTGGACCAGGGAATAC | |
| | TGGTACCCTGCCAGTCCCCCTGGAACACGCCCCTGCTACCCGTTAAG | |
| | AAACCAGGGACTAATGATTATAGGCCTGTCCAGGATCTGAGAGAAG | |
| | TCAACAAGCGGGTGGAAGACATCCACCCCACCGTGCCCAACCCTTAC | |
| | AACCTCTTGAGCGGGCCCCCACCGTCCCACCAGTGGTACACTGTGCT | |
| | TGATTTAAAGGATGCCTTTTTCTGCCTGAGACTCCACCCCACCAGTC | |
| | AGCCTCTCTTCGCCTTTGAGTGGAGAGATCCAGAGATGGGAATCTC | |
| | AGGACAATTGACCTGGACCAGACTCCCACAGGGTTTCAAAAACAGT | |
| | CCCACCCTGTTTAATGAGGCACTGCACAGAGACCTAGCAGACTTCCG | |
| | GATCCAGCACCCAGACTTGATCCTGCTACAGTACGTGGATGACTTAC | |
| | TGCTGGCCGCCACTTCTGAGCTAGACTGCCAACAAGGTACTCGGGCC | |
| | CTGTTACAAACCCTAGGGAACCTCGGGTATCGGGCCTCGGCCAAGAA | |
| | AGCCCAAATTTGCCAGAAACAGGTCAAGTATCTGGGGTATCTTCTA | |
| | AAAGAGGGTCAGAGATGGCTGACTGAGGCCAGAAAAGAGACTGTGA | |
| | TGGGGCAGCCTACTCCGAAGACCCCTCGACAACTAAGGGAGTTCCTA | |
| | GGGAAGGCAGGCTTCTGTCGCCTCTTCATCCCTGGGTTTGCAGAAAT | |
| | GGCAGCCCCCCTGTACCCTCTCACCAAACCGGGGACTCTGTTTAATT | |
| | GGGGCCCAGACCAACAAAAGGCCTATCAAGAAATCAAGCAAGCTCT | |
| | TCTAACTGCCCCAGCCCTGGGGTTGCCAGATTTGACTAAGCCCTTTG | |
| | AACTCTTTGTCGACGAGAAGCAGGGCTACGCCAAAGGTGTCCTAAC | |
| | GCAAAAACTGGGACCTTGGCGTCGGCCGGTGGCCTACCTGTCCAAAA | |
| | AGCTAGACCCAGTAGCAGCTGGGTGGCCCCCTTGCCTACGGATGGTA | |
| | GCAGCCATTGCCGTACTGACAAAGGATGCAGGCAAGCTAACCATGG | |
| | GACAGCCACTAGTCATTCTGGCCCCCCATGCAGTAGAGGCACTAGTC | |
| | AAACAACCCCCCGACCGCTGGCTTTCCAACGCCCGGATGACTCACTA | |
| | TCAGGCCTTGCTTTTGGACACGGACCGGGTCCAGTTCGGACCGGTGG | |
| | TAGCCCTGAACCCGGCTACGCTGCTCCCACTGCCTGAGGAAGGGCTG | |
| | CAACACAACTGCCTTGATATCCTGGCCGAAGCCCACGGAACCCGACC | |
| | CGACCTAACGGACCAGCCGCTCCCAGACGCCGACCACACCTGGTACA | |
| | CGGATGGAAGCAGTCTCTTACAAGAGGGACAGCGTAAGGCGGGAGC | |
| | TGCGGTGACCACCGAGACCGAGGTAATCTGGGCTAAAGCCCTGCCAG | |
| | CCGGGACATCCGCTCAGCGGGCTGAACTGATAGCACTCACCCAGGCC | |
| | CTAAAGATGGCAGAAGGTAAGAAGCTAAATGTTTATACTGATAGCC | |
| | GTTATGCTTTTGCTACTGCCCATATCCATGGAGAAATATACAGAAG | |
| | GCGTGGGTGGCTCACATCAGAAGGCAAAGAGATCAAAAATAAAGAC | |
| | GAGATCTTGGCCCTACTAAAAGCCCTCTTTCTGCCCAAAAGACTTAG | |
| | CATAATCCATTGTCCAGGACATCAAAAGGGACACAGCGCCGAGGCT | |
| | AGAGGCAACCGGATGGCTGACCAAGCGGCCCGAAAGGCAGCCATCA | |
| | CAGAGACTCCAGACACCTCTACCCTCCTCATAGAAAATTCATCACCC | |
| | TCTGGCGGCTCAAAAAGAACCGCCGACGGCAGCGAATTCGAGCCCA | |
| | AGAAGAAGAGGAAAGTCGGGGGGTCAGGTGGATCCGGCGGAAGTGG | |
| | CGGATCCGGTGGATCTGGCGGCAGTCCAAAAAAGAAAAGAAAAGTG | |
| | TATCCCTATGATGTCCCCGATTATGCCGGTTCAAGAGCCCTGGTCGT | |
| | GATTAGACTGAGCCGAGTGACAGACGCCACCACAAGTCCCGAGAGA | |
| | CAGCTGGAATCATGCCAGCAGCTCTGTGCTCAGCGGGGTTGGGATG | |
| | TGGTCGGCGTGGCAGAGGATCTGGACGTGAGCGGGGCCGTCGATCC | |
| | ATTCGACAGAAAGAGGAGGCCCAACCTGGCAAGATGGCTCGCTTTC | |
| | GAGGAACAGCCCTTTGATGTGATCGTCGCCTACAGAGTGGACCGGC | |
| | TGACCCGCTCAATTCGACATCTCCAGCAGCTGGTGCATTGGGCTGAG | |
| | GACCACAAGAAACTGGTGGTCAGCGCAACAGAAGCCCACTTCGATA | |
| | CTACCACACCTTTTGCCGCTGTGGTCATCGCACTGATGGGCACTGTG | |
| | GCCCAGATGGAGCTCGAAGCTATCAAGGAGCGAAACAGGAGCGCAG | |
| | CCCATTTCAATATTAGGGCCGGTAAATACAGAGGCTCCCTGCCCCCT | |
| | TGGGGATATCTCCCTACCAGGGTGGATGGGGAGTGGAGACTGGTGC | |
| | CAGACCCCGTCCAGAGAGAGCGGATTCTGGAAGTGTACCACAGAGT | |
| | GGTCGATAACCACGAACCACTCCATCTGGTGGCACACGACCTGAATA | |
| | GACGCGGCGTGCTCTCTCCAAAGGATTATTTTGCTCAGCTGCAGGGA | |
| | AGAGAGCCACAGGGAAGAGAATGGAGTGCTACTGCACTGAAGAGAT | |
| | CTATGATCAGTGAGGCTATGCTGGGTTACGCAACACTCAATGGCAA | |
| | AACTGTCCGGGACGATGACGGAGCCCCTCTGGTGAGGGCTGAGCCTA | |
| | TTCTCACCAGAGAGCAGCTCGAAGCTCTGCGGGCAGAACTGGTCAA | |
| | GACTAGTCGCGCCAAACCTGCCGTGAGCACCCCAAGCCTGCTCCTGA | |
| | GGGTGCTGTTCTGCGCCGTCTGTGGAGAGCCAGCATACAAGTTTGCC | |
| | GGCGGAGGGCGCAAACATCCCCGCTATCGATGCAGGAGCATGGGGT | |
| | TCCCTAAGCACTGTGGAAACGGGACAGTGGCCATGGCTGAGTGGGA | |
| | CGCCTTTTGCGAGGAACAGGTGCTGGATCTCCTGGGTGACGCTGAGC | |
| | GGCTGGAAAAAGTGTGGGTGGCAGGATCTGACTCCGCTGTGGAGCT | |
| | GGCAGAAGTCAATGCCGAGCTCGTGGATCTGACTTCCCTCATCGGAT | |
| | CTCCTGCATATAGAGCTGGGTCCCCACAGAGAGAAGCTCTGGACGCA | |
| | CGAATTGCTGCACTCGCTGCTAGACAGGAGGAACTGGAGGGCCTGG | |
| | AGGCCAGGCCCTCTGGATGGGAGTGGCGAGAAACCGGACAGAGGTT | |
| | TGGGGATTGGTGGAGGGAGCAGGACACCGCAGCCAAGAACACATGG | |
| | CTGAGATCCATGAATGTCCGGCTCACATTCGACGTGCGCGGTGGCCT | |
| | GACTCGAACCATCGATTTTGGCGACCTGCAGGAGTATGAACAGCAC | |
| | CTGAGACTGGGGTCCGTGGTCGAAAGACTGCACACTGGGATGTCCT | |
| | AG | |

The exogenous polynucleotide is provided in Table 17.

TABLE 17
Nucleic acid sequence of exogenous polynucleotide.

| | | SEQ |
|---|---|---|
| | | ID |
| Description | Nucleic Acid Sequence | NO: |
| Exogenous | GTGGTTTGTCTGGTCAACCACCGCGGTCTCAGTGGTGTACGGTACAA | 272 |
| polynucleotide | ACCCAGCTACCGGTCGCCACCATGCCCGCCATGAAGATCGAGTGCCG | |
| | CATCACCGGCACCCTGAACGGCGTGGAGTTCGAGCTGGTGGGCGGCG | |
| | GAGAGGGCACCCCCGAGCAGGGCCGCATGACCAACAAGATGAAGAG | |
| | CACCAAAGGCGCCCTGACCTTCAGCCCCTACCTGCTGAGCCACGTGA | |
| | TGGGCTACGGCTTCTACCACTTCGGCACCTACCCCAGCGGCTACGAG | |
| | AACCCCTTCCTGCACGCCATCAACAACGGCGGCTACACCAACACCCG | |
| | CATCGAGAAGTACGAGGACGGCGGCGTGCTGCACGTGAGCTTCAGC | |
| | TACCGCTACGAGGCCGGCCGCGTGATCGGCGACTTCAAGGTGGTGGG | |
| | CACCGGCTTCCCCGAGGACAGCGTGATCTTCACCGACAAGATCATCC | |
| | GCAGCAACGCCACCGTGGAGCACCTGCACCCCATGGGCGATAACGTG | |
| | CTGGTGGGCAGCTTCGCCCGCACCTTCAGCCTGCGCGACGGCGGCTA | |
| | CTACAGCTTCGTGGTGGACAGCCACATGCACTTCAAGAGCGCCATCC | |
| | ACCCCAGCATCCTGCAGAACGGGGGCCCCATGTTCGCCTTCCGCCGC | |
| | GTGGAGGAGCTGCACAGCAACACCGAGCTGGGCATCGTGGAGTACC | |
| | AGCACGCCTTCAAGACCCCCATCGCCTTCGCCAGATCTCGAGCTCGA | |
| | TGAGTTTGGACAAACCACAACTAGAATGCAGTGAAAAAAATGCTTT | |
| | ATTTGTGAAATTTGTGATGCTATTGCTTTATTTGTgggcccgccccaact | |
| | ggggtaacctttgagttctctcagttgggg | |

Integration of the exogenous polynucleotide by the Bxb1 integrase is determined by genomic DNA harvesting and targeted amplicon next generation sequencing. Briefly, DNA is harvested from transfected cells by removal of the culture media, resuspension of the cells in 50 μL of QuickExtract (Lucigen), and incubation at 65° C. for 15 min, 68° C. for 15 min, and 98° C. for 10 min. Target genomic regions are PCR amplified with NEBNext High-Fidelity 2×PCR Master Mix (NEB) according to the manufacturer's protocol. Barcodes and adapters for Illumina sequencing are added in a subsequent PCR amplification. Amplicons are pooled and prepared for sequencing on a MiSeq (Illumina). Reads are demultiplexed and analyzed with appropriate pipelines.

This example shows that use of a separate ttRNA and target gRNA within the PASTE editing system mediates nucleic acid integration of an exogenous polynucleotide at two different target sites (LMNB1 and ACTB). The integration requires the addition of an aptamer binding protein to the editing polypeptide to recruit the ttRNA with the corresponding aptamer to the target site. Overall, this example shows that the classical pegRNA utilized in PRIME editing, can be split into two molecules, a targeting gRNA targeting the Cas9 nickase to the target site and a ttRNA comprising the primer binding site, the reverse transcriptase template sequence, and the integration sequence, while maintaining efficient PASTE integration.

The disclosure is not to be limited in scope by the specific embodiments described herein. Indeed, various modifications will become apparent to those skilled in the art from the foregoing description and accompanying FIGURES. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

1. An editing polypeptide that comprises (i) a DNA binding nickase, (ii) a reverse transcriptase, (iii) an aptamer binding protein; and (iv) an integrase;
wherein each of said DNA binding nickase, reverse transcriptase, aptamer binding protein, and integrase, are each operably connected in any order by a linker.

2.-4. (canceled)

5. The editing polypeptide of claim 1, wherein said aptamer binding protein is an MS2 coat protein (MCP), a Qβ coat protein, or a PP7 coat protein, or a functional fragment or variant thereof.

6. (canceled)

7. The editing polypeptide of claim 1, wherein said integrase is Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), or Minos, or any functional fragments or variants thereof.

8. The editing polypeptide of claim 1, wherein said DNA binding nickase is a Cas9-D10A, a Cas9-H840A, a Cas12a nickase, or a Cas12b nickase, or a functional fragment or variant thereof.

9. The editing polypeptide of claim 1, wherein said reverse transcriptase is derived from a Moloney Murine Leukemia Virus (M-MLV) reverse transcriptase domain, transcription xenopolymerase (RTX), avian myeloblastosis virus reverse transcriptase (AMV-RT), or Eubacterium rectale maturase RT (MarathonRT).

10-17. (canceled)

18. An RNA polynucleotide comprising (i) a primer binding site, (ii) a reverse transcription template sequence that comprises an integration recognition sequence, and (iii) at least one aptamer.

19. The RNA polynucleotide of claim 18, wherein said aptamer is an MS2 aptamer, a Qβ RNA aptamer, or a PP7 RNA aptamer.

20. (canceled)

21. The RNA polynucleotide of claim 18, wherein said integration recognition sequence comprises an attB site, an attP site, an attL site, an attR site, a Vox site, or a FRT site.

22.-29. (canceled)

30. A composition comprising:
(a) a polynucleotide encoding an editing polypeptide comprising:
(i) a DNA binding nickase (or a functional fragment or variant thereof), (ii) a reverse transcriptase (or a functional fragment or variant thereof), (iii) an aptamer binding protein (or functional fragment or variant thereof); and (iv) an integrase (or a functional fragment or variant thereof);
(b) a trans-template RNA (ttRNA) polynucleotide comprising:
(i) a primer binding site, (ii) a reverse transcription template sequence that comprises an integration recognition sequence, and (iii) at least one aptamer; and
(c) at least one targeting guide RNA (gRNA) comprising:
(i) a spacer and (ii) a scaffold.

31.-34. (canceled)

35. The composition of claim 30, further comprising (d) a nicking gRNA (ngRNA).

36.-45. (canceled)

46. A method of site-specifically integrating a polynucleotide of interest into a target dsDNA polynucleotide, the method comprising:
(1) incorporating an integration recognition sequence into a target location in said target dsDNA polynucleotide by contacting said target dsDNA polynucleotide with:
(a) an editing polypeptide comprising: (i) a DNA binding nickase, (ii) a reverse transcriptase, (iii) an aptamer binding protein, and (iv) an integrase, wherein each of said DNA binding nickase, reverse transcriptase, aptamer binding protein, and integrase are each operably connected in any order;
(b) a targeting guide RNA (gRNA) comprising (i) a spacer and (ii) a scaffold; and
(c) a trans-template RNA (ttRNA) comprising (i) a primer binding site, (ii) a reverse transcription template sequence that comprises an integration recognition sequence, and (iii) an aptamer;
wherein said editing polypeptide's DNA binding nickase nicks a strand of said target dsDNA polynucleotide, and said reverse transcriptase reverse transcribes the reverse transcription template sequence into an extended sequence that encodes the first integration recognition sequence or a complement thereof and the extended sequence is incorporated; and
(2) integrating said polynucleotide of interest into said target dsDNA polynucleotide by contacting said target dsDNA polynucleotide with a polynucleotide that comprises said polynucleotide of interest operably connected to a polynucleotide that comprises a sequence complementary or associated to said integration recognition sequence;
wherein said integrase incorporates said polynucleotide of interest into said target dsDNA polynucleotide by integration of said sequence that is complementary or associated to said integration recognition sequence to thereby site-specifically integrate said polynucleotide of interest into said target dsDNA polynucleotide.

47. A method of site-specifically integrating of a polynucleotide of interest into a target dsDNA polynucleotide, the method comprising:
(1) incorporating an integration recognition sequence into a target location in said target dsDNA polynucleotide by contacting said target dsDNA polynucleotide with:
(a) an editing polypeptide comprising: (i) a DNA binding nickase, (ii) a reverse transcriptase, and (iii) an aptamer binding protein, wherein each of said DNA binding nickase, reverse transcriptase, and aptamer binding protein, are each operably connected in any order via a linker;
(b) a targeting guide RNA (gRNA) comprising: (i) a spacer and (ii) a scaffold; and
(c) a trans-template RNA (ttRNA) comprising: (i) a primer binding site, (ii) a reverse transcription template sequence that comprises an integration recognition sequence, and (iii) an aptamer;
wherein said editing polypeptide's DNA binding nickase nicks a strand of said target dsDNA polynucleotide, and said reverse transcriptase reverse transcribes the reverse transcription template sequence into an extended sequence that encodes the first integration recognition sequence or a complement thereof and the first extended sequence is incorporated into said target location in said target dsDNA polynucleotide; and
(2) integrating said polynucleotide of interest into said target dsDNA polynucleotide by contacting said target dsDNA polynucleotide with an integrase or a functional fragment or variant thereof and a polynucleotide that comprises said polynucleotide of interest operably connected to a polynucleotide that comprises a sequence complementary or associated to said integration recognition sequence;
wherein said integrase incorporates said polynucleotide of interest into said target dsDNA polynucleotide by integration of the sequence that is complementary or associated to said integration recognition sequence to thereby site-specifically integrate said polynucleotide of interest into said target dsDNA polynucleotide.

48. The method of claim 47, wherein said aptamer binding protein is an MS2 coat protein (MCP), a Qβ coat protein, or a PP7 coat protein, or a functional fragment or variant thereof.

49.-51. (canceled)

52. The method of claim 47, wherein said integrase is Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), or Minos, or any functional fragments or variants thereof.

53. The method of claim 47, wherein said DNA binding nickase is a Cas9-D10A, a Cas9-H840A, a Cas12a, or a Cas12b nickase, or a functional fragment or variant thereof.

54. The method of claim 47, wherein said reverse transcriptase is derived from a Moloney Murine Leukemia Virus (M-MLV) reverse transcriptase domain, transcription xenopolymerase (RTX), avian myeloblastosis virus reverse transcriptase (AMV-RT), or Eubacterium rectale maturase RT (MarathonRT).

55-57. (canceled)

58. The method of claim 47, wherein said polynucleotide of interest comprises one or more nucleotide modification comprising insertion, deletion or substitution compared to the endogenous sequence of said target dsDNA polynucleotide.

59.-67. (canceled)

68. The method of claim 47, further comprising contacting said dsDNA polynucleotide with a ngRNA.

69.-71. (canceled)

72. A method of site-specifically integrating of a polynucleotide of interest into a target dsDNA polynucleotide in a cell, the method comprising:
(1) incorporating an integration recognition sequence into a target location in a target dsDNA polynucleotide in a cell by introducing into a cell:
(a)(i) an editing polypeptide comprising a DNA binding nickase (or a functional fragment or variant thereof), a reverse transcriptase (or a functional fragment or variant thereof), an aptamer binding protein (or a functional fragment or variant thereof), and an integrase (or a functional fragment or variant thereof), wherein each of said DNA binding nickase, reverse transcriptase, aptamer binding protein, and integrase are each operably connected in any order via a linker; or (a)(ii) a polynucleotide encoding the editing polypeptide of (a)(i);
(b) a targeting gRNA comprising (i) a spacer and (ii) a scaffold; and
(c) a trans-template RNA (ttRNA) comprising (i) a primer binding site, (ii) a reverse transcription template sequence that comprises an integration sequence that comprises an integration recognition sequence, and (iii) an aptamer;
wherein said editing polypeptide's DNA binding nickase nicks a strand of said target dsDNA polynucleotide, and said reverse transcriptase incorporates said integration sequence into said target dsDNA polynucleotide, thereby incorporating said integration recognition sequence into said target location in said target dsDNA polynucleotide; and
(2) integrating said polynucleotide of interest into said target dsDNA polynucleotide by introducing into said cell a polynucleotide that comprises said polynucleotide of interest operably connected to a polynucleotide that comprises a sequence complementary or associated to said integration recognition sequence;
wherein said integrase incorporates said polynucleotide of interest into said target dsDNA polynucleotide by integration, recombination, or reverse transcription of said sequence that is complementary or associated to said integration recognition sequence to thereby site-specifically integrate said polynucleotide of interest into said target dsDNA polynucleotide in a cell.

73. A method of site-specifically integrating of a polynucleotide of interest into a target dsDNA polynucleotide in a cell, the method comprising:
(1) incorporating an integration recognition sequence into a target location in a target dsDNA polynucleotide in a cell by introducing into a cell:
(a)(i) an editing polypeptide comprising a DNA binding nickase (or a functional fragment or variant thereof), a reverse transcriptase (or a functional fragment or variant thereof), and an aptamer binding protein (or a functional fragment or variant thereof), wherein each of said DNA binding nickase, reverse transcriptase, and aptamer binding protein, are each operably connected in any order; or (a)(ii) a polynucleotide encoding the editing polypeptide of (a)(i);
(b) a targeting gRNA comprising (i) a spacer and (ii) a scaffold; and
(c) a trans-template RNA (ttRNA) comprising (i) a primer binding site, (ii) a reverse transcription template sequence that comprises an integration sequence that comprises an integration recognition sequence, and (iii) an aptamer;
wherein said editing polypeptide's DNA binding nickase nicks a strand of said target dsDNA polynucleotide, and said reverse transcriptase reverse transcribes the reverse transcription template sequence into an extended sequence that encodes the first integration recognition sequence or a complement thereof and the first extended sequence is incorporated into said target location in said target dsDNA polynucleotide; and
(2) integrating said polynucleotide of interest into said target dsDNA polynucleotide by introducing into said cell an integrase or a functional fragment or variant thereof and a polynucleotide that comprises said polynucleotide of interest operably connected to a polynucleotide that comprises a sequence complementary or associated to said integration recognition sequence;
wherein said integrase incorporates said polynucleotide of interest into said target dsDNA polynucleotide by integration, recombination, or reverse transcription of said sequence that is complementary or associated to said integration recognition sequence to thereby site-specifically integrate said polynucleotide of interest into said target dsDNA polynucleotide in a cell.
